(19) United States
(12) Patent Application Publication
(10) Pub. No.: US 2022/0411479 A1
(43) Pub. Date: Dec. 29, 2022

(54) CD20 CHIMERIC ANTIGEN RECEPTORS AND METHODS OF USE FOR IMMUNOTHERAPY

(71) Applicant: Precision BioSciences, Inc., Durham, NC (US)

(72) Inventors: Daniel T. MacLeod, La Jolla, CA (US); Bruce J. McCreedy, JR., Raleigh, NC (US); Aaron Martin, Carrboro, NC (US)

(73) Assignee: Precision BioSciences, Inc., Durham, NC (US)

(21) Appl. No.: 17/773,456

(22) PCT Filed: Oct. 30, 2020
(86) PCT No.: PCT/US2020/058279

§ 371 Date: Apr. 29, 2022

Related U.S. Application Data
(60) Provisional application No. 63/019,125, filed on May 1, 2020.
(60) Provisional application No. 62/928,344, filed on Oct. 30, 2019.

(51) Int. Cl. C07K 14/725; A61K 35/17; A61K 45/06; A61P 35/00

(52) U.S. Cl. CPC C07K 14/7051; A61K 35/17; A61K 45/06; A61P 35/00; A61K 38/00

(57) ABSTRACT

Provided herein are compositions and methods for the treatment of a disease, such as cancer, using a chimeric antigen receptor or genetically-modified cells comprising a chimeric antigen receptor having specificity for CD20. The invention provides polynucleotides encoding such chimeric antigen receptors, and genetically-modified cells comprising such chimeric antigen receptors. Also provided are methods for making such genetically-modified cells and pharmaceutical compositions comprising the same. The invention further provides methods for treating a disease (e.g., cancer) in a subject by administering such genetically-modified cells or compositions. The main embodiments concern CARs with an scFv specific for CD20, the hinge and transmembrane domains from CD8, the costimulatory cytoplasmic or signalling domain from co-stimulatory molecules Novell (N1) or Novel6 (N6) and the CD3zeta intracellular signaling domain.

### FIELD OF THE INVENTION

The present disclosure provides polynucleotides encoding chimeric antigen receptors, genetically-modified cells expressing chimeric antigen receptors, and pharmaceutical compositions thereof. Also provided are methods of using genetically-modified cells expressing chimeric antigen receptors, and pharmaceutical compositions thereof, for the treatment of cancer and other disorders and diseases.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Oct. 30, 2020, is named P1090.70045WO00-SEQ-EPG, and is 121 kilobytes in size.

### BACKGROUND OF THE INVENTION

T cell adoptive immunotherapy is a promising approach for cancer treatment. This strategy utilizes isolated human T cells that have been genetically-modified to enhance their specificity for a specific tumor associated antigen. Genetic modification may involve the expression of a chimeric antigen receptor (CAR) or an exogenous T cell receptor to graft antigen specificity onto the T cell. By contrast to exogenous T cell receptors, CARs derive their specificity from the variable domains of a monoclonal antibody. Thus, T cells expressing CARs (CAR T cells) induce tumor immunoreactivity in a major histocompatibility complex non-restricted manner. To date, T cell adoptive immunotherapy has been utilized as a clinical therapy for a number of cancers, including B cell malignancies (e.g., acute lymphoblastic leukemia (ALL), B cell non-Hodgkin lymphoma (NHL), and chronic lymphocytic leukemia), multiple myeloma, neuroblastoma, glioblastoma, advanced gliomas, ovarian cancer, mesothelioma, melanoma, and pancreatic cancer.

CAR clinical trials for NHL have largely targeted antigens including CD19, CD20, and CD22, which are expressed on both malignant lymphoid cells and normal B cells. CD20 is an attractive target for CAR T therapy due to its widespread clinical success as an immunotherapy target, particularly in clinical trials with the anti-CD20 monoclonal antibody rituximab. While many clinical trials have focused on the use of CD19 as a target antigen, CD19 is internalized following antibody binding, and loss of CD19 expression on the cell surface has been postulated to be a mechanism by which cancer cells can escape eradication by anti-CD19 CAR T therapy. Pursuit of an anti-CD20 CAR T therapy can allow for either concurrent or sequential anti-CD19/anti-CD20 CAR T therapy to address issues of CD19 antigen escape.

There is a need in the field of immunotherapy for additional compositions and methods useful for the treatment of CD20-expressing cancers.

### SUMMARY OF THE INVENTION

Accordingly, described herein are novel CARs that have specificity against CD20 epitopes. Further described herein are genetically-modified cells expressing the CARs according to the invention (e.g., CAR T cells) and novel methods of treating a CD20 related disease (e.g., a cancer) with the genetically-modified cells. In addition, described herein are methods for manufacturing the genetically-modified cells and pharmaceutical compositions and kits comprising the genetically-modified cells according to the invention.

In one aspect, the invention provides a polynucleotide encoding a chimeric antigen receptor, wherein said polynucleotide comprises a nucleic acid sequence having at least 80%, at least 85%, at least 90%, at least 95%, or more, sequence identity to a nucleic acid sequence set forth in any one of SEQ ID NO: 40, SEQ ID NO: 42 SEQ ID NO: 44, SEQ ID NO: 46, SEQ ID NO: 52, SEQ ID NO: 54, SEQ ID NO: 56, and SEQ ID NO: 58. In some embodiments, the chimeric antigen receptor comprises a single chain variable fragment (scFv), a hinge domain, a transmembrane domain, a co-stimulatory domain, and a signaling domain.

In some embodiments, the nucleic acid sequence is set forth in SEQ ID NO: 40. In some embodiments, the nucleic acid sequence is set forth in SEQ ID NO: 42. In some embodiments, the nucleic acid sequence is set forth in SEQ ID NO: 44. In some embodiments, the nucleic acid sequence is set forth in SEQ ID NO: 46. In some embodiments, the nucleic acid sequence is set forth in SEQ ID NO: 52. In some embodiments, the nucleic acid sequence is set forth in SEQ ID NO: 54. In some embodiments, the nucleic acid sequence is set forth in SEQ ID NO: 56. In some embodiments, the nucleic acid sequence is set forth in SEQ ID NO: 58.

In some embodiments, the polynucleotide further encodes a signal peptide. In some embodiments, the signal peptide comprises a nucleic acid sequence having at least 80%, at least 85%, at least 90%, at least 95%, or more, sequence identity to SEQ ID NO: 34. In some embodiments, the signal peptide comprises a nucleic acid sequence set forth in SEQ ID NO: 34.

In some embodiments, the polynucleotide is an mRNA. In some embodiments, the polynucleotide is a recombinant DNA construct. In some embodiments, the polynucleotide is comprised by a virus (i.e., a viral vector). In some such embodiments, the virus is an adenovirus (i.e., an adenoviral vector), a lentivirus (i.e., a lentiviral vector), a retrovirus (i.e., a retroviral vector), or an adeno-associated virus (AAV) (i.e., an AAV vector). In particular embodiments, the viral vector can be a recombinant AAV (i.e., a recombinant AAV vector). In other embodiments, the polynucleotide is a double-stranded DNA sequence integrated into the genome of a cell.

In another aspect, the invention provides a polynucleotide encoding a chimeric antigen receptor having specificity for CD20, wherein the chimeric antigen receptor comprises: (a) a single chain variable fragment (scFv) having specificity for CD20, wherein the scFv comprises: (i) a heavy chain variable (VH) domain comprising a CDRH1, a CDRH2, and a CDRH3 set forth in SEQ ID NO: 1, a polypeptide linker, and a light chain variable (VL) domain comprising a CDRL1, a CDRL2, and a CDRL3 set forth in SEQ ID NO: 3; or (ii) a VH domain comprising a CDRH1, a CDRH2, and a CDRH3 set forth in SEQ ID NO: 5, a polypeptide linker, and a VL domain comprising a CDRL1, a CDRL2, and a CDRL3 set forth in SEQ ID NO: 7; (b) a hinge domain; (c) a transmembrane domain; (d) a co-stimulatory domain having at least 80%, at least 85%, at least 90%, at least 95%, at least 96%, at least 97%, at least 98%, at least 99%, or more, sequence identity to SEQ ID NO: 21 or SEQ ID NO: 23; and (e) a signaling domain.

In some embodiments, the chimeric antigen receptor comprises: (a) a single chain variable fragment (scFv) having specificity for CD20, wherein the scFv comprises: (i) a heavy chain variable (VH) domain comprising a CDRH1, a CDRH2, and a CDRH3 set forth in SEQ ID NO: 1, a polypeptide linker, and a light chain variable (VL) domain comprising a CDRL1, a CDRL2, and a CDRL3 set forth in SEQ ID NO: 3; or (ii) a VH domain comprising a CDRH1, a CDRH2, and a CDRH3 set forth in SEQ ID NO: 5, a polypeptide linker, and a VL domain comprising a CDRL1, a CDRL2, and a CDRL3 set forth in SEQ ID NO: 7; (b) a hinge domain; (c) a transmembrane domain; (d) a co-stimulatory domain having at least 80%, at least 85%, at least 90%, at least 95%, at least 96%, at least 97%, at least 98%, at least 99%, or 100% sequence identity to SEQ ID NO: 21; and (e) a signaling domain.

In some embodiments, the chimeric antigen receptor comprises: (a) a single chain variable fragment (scFv) having specificity for CD20, wherein the scFv comprises: (i) a heavy chain variable (VH) domain comprising a CDRH1, a CDRH2, and a CDRH3 set forth in SEQ ID NO: 1, a polypeptide linker, and a light chain variable (VL) domain comprising a CDRL1, a CDRL2, and a CDRL3 set forth in SEQ ID NO: 3; or (ii) a VH domain comprising a CDRH1, a CDRH2, and a CDRH3 set forth in SEQ ID NO: 5, a polypeptide linker, and a VL domain comprising a CDRL1, a CDRL2, and a CDRL3 set forth in SEQ ID NO: 7; (b) a hinge domain; (c) a transmembrane domain; (d) a co-stimulatory domain having at least 80%, at least 85%, at least 90%, at least 95%, at least 96%, at least 97%, at least 98%, at least 99%, or 100% sequence identity to SEQ ID NO: 23; and (e) a signaling domain.

In some embodiments, the CDRs are defined by the Kabat numbering scheme. In some such embodiments, the VH domain comprises a CDRH1 of SEQ ID NO: 9, a CDRH2 of SEQ ID NO: 10, and a CDRH3 of SEQ ID NO: 11, and the VL domain comprises a CDRL1 of SEQ ID NO: 12, a CDRL2 of SEQ ID NO: 13, and a CDRL3 of SEQ ID NO: 14.

In other embodiments, wherein the CDRs are defined by the Kabat numbering scheme, the VH domain comprises a CDRH1 of SEQ ID NO: 15, a CDRH2 of SEQ ID NO: 16, and a CDRH3 of SEQ ID NO: 17, and the VL domain comprises a CDRL1 of SEQ ID NO: 18, a CDRL2 of SEQ ID NO: 19, and a CDRL3 of SEQ ID NO: 20.

In some embodiments, the polypeptide linker comprises an amino acid sequence having at least 80%, at least 85%, at least 90%, at least 95%, or more, sequence identity to SEQ ID NO: 25 or SEQ ID NO: 71. In some embodiments, the polypeptide linker comprises an amino acid sequence of SEQ ID NO: 25. In some embodiments, the polypeptide linker comprises an amino acid sequence of SEQ ID NO: 71. In some embodiments, the hinge domain comprises an amino acid sequence having at least 80%, at least 85%, at least 90%, at least 95%, or more, sequence identity to SEQ ID NO: 27. In some embodiments, the hinge domain comprises an amino acid sequence of SEQ ID NO: 27. In some embodiments, the transmembrane domain comprises an amino acid sequence having at least 80%, at least 85%, at least 90%, at least 95%, or more, sequence identity to SEQ ID NO: 29. In some embodiments, the transmembrane domain comprises an amino acid sequence of SEQ ID NO: 29. In some embodiments, the signaling domain comprises an amino acid sequence having at least 80%, at least 85%, at least 90%, at least 95%, or more, sequence identity to SEQ ID NO: 31. In some embodiments, the signaling domain comprises an amino acid sequence of SEQ ID NO: 31.

In some embodiments, the VH domain has at least 80%, at least 85%, at least 90%, at least 95%, or more, sequence identity to SEQ ID NO: 1; and the VL domain has at least 80%, at least 85%, at least 90%, at least 95%, or more, sequence identity to SEQ ID NO: 3. In some embodiments, the VH domain comprises SEQ ID NO: 1; and the VL domain comprises SEQ ID NO: 3. In some embodiments, the scFv comprises an amino acid sequence having at least 80%, at least 85%, at least 90%, at least 95%, or more, sequence identity to SEQ ID NO: 35 or SEQ ID NO: 37. In some embodiments, the scFv comprises an amino acid of SEQ ID NO: 35 or SEQ ID NO: 37. In some embodiments, he scFv comprises an amino acid sequence of SEQ ID NO: 35. In some embodiments, he scFv comprises an amino acid sequence of SEQ ID NO: 37. In some embodiments, the co-stimulatory domain comprises an amino acid sequence of SEQ ID NO: 21. In some such embodiments, the chimeric antigen receptor comprises an amino acid sequence having at least 80%, at least 85%, at least 90%, at least 95%, or more, sequence identity to SEQ ID NO: 39 or SEQ ID NO: 41. In some such embodiments, the chimeric antigen receptor comprises an amino acid sequence of SEQ ID NO: 39 or SEQ ID NO: 41. In some such embodiments, the chimeric antigen receptor comprises an amino acid sequence of SEQ ID NO: 39. In some such embodiments, the chimeric antigen receptor comprises an amino acid sequence of SEQ ID NO: 41. In other embodiments, the co-stimulatory domain comprises an amino acid sequence of SEQ ID NO: 23. In some such embodiments, the chimeric antigen receptor comprises an amino acid sequence having at least 80%, at least 85%, at least 90%, at least 95%, or more, sequence identity to SEQ ID NO: 43 or SEQ ID NO: 45. In some such embodiments, the chimeric antigen receptor comprises an amino acid sequence of SEQ ID NO: 43 or SEQ ID NO: 45. In some embodiments, the chimeric antigen receptor comprises an amino acid sequence of SEQ ID NO: 43. In some embodiments, the chimeric antigen receptor comprises an amino acid sequence of SEQ ID NO: 45.

In some embodiments, the VH domain has at least 80%, at least 85%, at least 90%, at least 95%, or more, sequence identity to SEQ ID NO: 5; and the VL domain has at least 80%, at least 85%, at least 90%, at least 95%, or more, sequence identity to SEQ ID NO: 7. In some embodiments, the VH domain comprises SEQ ID NO: 5; and the VL domain comprises SEQ ID NO: 7. In some embodiments, the scFv comprises an amino acid sequence having at least 80%, at least 85%, at least 90%, at least 95%, or more, sequence identity to SEQ ID NO: 47 or SEQ ID NO: 49. In some embodiments, the scFv comprises an amino acid of SEQ ID NO: 47 or SEQ ID NO: 49. In some embodiments, the co-stimulatory domain comprises an amino acid sequence of SEQ ID NO: 21. In some such embodiments, the chimeric antigen receptor comprises an amino acid sequence having at least 80%, at least 85%, at least 90%, at least 95%, or more sequence identity to SEQ ID NO: 51 or SEQ ID NO: 53. In some such embodiments, the chimeric antigen receptor comprises an amino acid sequence of SEQ ID NO: 51 or SEQ ID NO: 53. In some such embodiments, the chimeric antigen receptor comprises an amino acid sequence of SEQ ID NO: 51. In some such embodiments, the chimeric antigen receptor comprises an amino acid sequence of SEQ ID NO: 53. In other embodiments, the co-stimulatory domain comprises an amino acid sequence of SEQ ID NO: 23. In some such embodiments, the chimeric antigen receptor comprises an amino acid sequence having at least 80%, at least 85%, at least 90%, at least 95%, or more sequence identity to SEQ ID NO: 55 or SEQ ID NO: 57. In some such embodiments, the chimeric antigen receptor comprises an amino acid sequence of SEQ ID NO: 55 or SEQ ID NO: 57. In some embodiments, the chimeric antigen receptor comprises an amino acid sequence of SEQ ID NO: 55. In some embodiments, the chimeric antigen receptor comprises an amino acid sequence of SEQ ID NO: 57.

In some embodiments, the chimeric antigen receptor comprises: (a) a single chain variable fragment (scFv) having specificity for CD20, wherein the scFv comprises: (i) a heavy chain variable (VH) domain comprising a CDRH1, a CDRH2, and a CDRH3 set forth in SEQ ID NO: 1, a polypeptide linker, and a light chain variable (VL) domain comprising a CDRL1, a CDRL2, and a CDRL3 set forth in SEQ ID NO: 3; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:21; and (e) a signaling domain comprising an amino acid sequence having at least 95%, preferably 100%, sequence identity to SEQ ID NO:31. In some embodiments, the CDRs are defined by the Kabat numbering scheme. In some such embodiments, the VH domain comprises a CDRH1 of SEQ ID NO: 9, a CDRH2 of SEQ ID NO: 10, and a CDRH3 of SEQ ID NO: 11, and the VL domain comprises a CDRL1 of SEQ ID NO: 12, a CDRL2 of SEQ ID NO: 13, and a CDRL3 of SEQ ID NO: 14. In some such embodiments, the VH domain has at least 95%, preferably 100%, sequence identity to SEQ ID NO: 1, and the VL domain has at least 95%, preferably 100%, sequence identity to SEQ ID NO: 3. In some such embodiments, the scFv comprises an amino acid sequence having at least 95%, preferably 100%, sequence identity to SEQ ID NO: 35. In some such embodiments, the scFv comprises an amino acid sequence having at least 95%, preferably 100%, sequence identity to SEQ ID NO: 37. In some such embodiments, the chimeric antigen receptor comprises an amino acid sequence having at least 95%, preferably 100%, sequence identity to SEQ ID NO: 39. In some such embodiments, the chimeric antigen receptor comprises an amino acid sequence having at least 95%, preferably 100%, sequence identity to SEQ ID NO: 41.

In some embodiments, the chimeric antigen receptor comprises: (a) a single chain variable fragment (scFv) having specificity for CD20, wherein the scFv comprises: (i) a heavy chain variable (VH) domain comprising a CDRH1, a CDRH2, and a CDRH3 set forth in SEQ ID NO: 1, a polypeptide linker, and a light chain variable (VL) domain comprising a CDRL1, a CDRL2, and a CDRL3 set forth in SEQ ID NO: 3; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:23; and (e) a signaling domain comprising an amino acid sequence having at least 95%, preferably 100%, sequence identity to SEQ ID NO:31. In some embodiments, the CDRs are defined by the Kabat numbering scheme. In some such embodiments, the VH domain comprises a CDRH1 of SEQ ID NO: 9, a CDRH2 of SEQ ID NO: 10, and a CDRH3 of SEQ ID NO: 11, and the VL domain comprises a CDRL1 of SEQ ID NO: 12, a CDRL2 of SEQ ID NO: 13, and a CDRL3 of SEQ ID NO: 14. In some such embodiments, the VH domain has at least 95%, preferably 100%, sequence identity to SEQ ID NO: 1, and the VL domain has at least 95%, preferably 100%, sequence identity to SEQ ID NO: 3. In some such embodiments, the scFv comprises an amino acid sequence having at least 95%, preferably 100%, sequence identity to SEQ ID NO: 35. In some such embodiments, the scFv comprises an amino acid sequence having at least 95%, preferably 100%, sequence identity to SEQ ID NO: 37. In some such embodiments, the chimeric antigen receptor comprises an amino acid sequence having at least 95%, preferably 100%, sequence identity to SEQ ID NO: 43. In some such embodiments, the chimeric antigen receptor comprises an amino acid sequence having at least 95%, preferably 100%, sequence identity to SEQ ID NO: 45.

In some embodiments, the chimeric antigen receptor comprises: (a) a single chain variable fragment (scFv) having specificity for CD20, wherein the scFv comprises: (i) a heavy chain variable (VH) domain comprising a CDRH1, a CDRH2, and a CDRH3 set forth in SEQ ID NO: 5, a polypeptide linker, and a light chain variable (VL) domain comprising a CDRL1, a CDRL2, and a CDRL3 set forth in SEQ ID NO: 7; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:21; and (e) a signaling domain comprising an amino acid sequence having at least 95%, preferably 100%, sequence identity to SEQ ID NO:31. In some embodiments, the CDRs are defined by the Kabat numbering scheme. In some such embodiments, the VH domain comprises a CDRH1 of SEQ ID NO: 15, a CDRH2 of SEQ ID NO: 16, and a CDRH3 of SEQ ID NO: 17, and the VL domain comprises a CDRL1 of SEQ ID NO: 18, a CDRL2 of SEQ ID NO: 19, and a CDRL3 of SEQ ID NO: 20. In some such embodiments, the VH domain has at least 95%, preferably 100%, sequence identity to SEQ ID NO: 5, and the VL domain has at least 95%, preferably 100%, sequence identity to SEQ ID NO: 7. In some such embodiments, the scFv comprises an amino acid sequence having at least 95%, preferably 100%, sequence identity to SEQ ID NO: 47. In some such embodiments, the scFv comprises an amino acid sequence having at least 95%, preferably 100%, sequence identity to SEQ ID NO: 49. In some such embodiments, the chimeric antigen receptor comprises an amino acid sequence having at least 95%, preferably 100%, sequence identity to SEQ ID NO: 51. In some such embodiments, the chimeric antigen receptor comprises an amino acid sequence having at least 95%, preferably 100%, sequence identity to SEQ ID NO: 53.

In some embodiments, the chimeric antigen receptor comprises: (a) a single chain variable fragment (scFv) having specificity for CD20, wherein the scFv comprises: (i) a heavy chain variable (VH) domain comprising a CDRH1, a CDRH2, and a CDRH3 set forth in SEQ ID NO: 5, a polypeptide linker, and a light chain variable (VL) domain comprising a CDRL1, a CDRL2, and a CDRL3 set forth in SEQ ID NO: 7; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:23; and (e) a signaling domain comprising an amino acid sequence having at least 95%, preferably 100%, sequence identity to SEQ ID NO:31. In some embodiments, the CDRs are defined by the Kabat numbering scheme. In some such embodiments, the VH domain comprises a CDRH1 of SEQ ID NO: 15, a CDRH2 of SEQ ID NO: 16, and a CDRH3 of SEQ ID NO: 17, and the VL domain comprises a CDRL1 of SEQ ID NO: 18, a CDRL2 of SEQ ID NO: 19, and a CDRL3 of SEQ ID NO: 20. In some such embodiments, the VH domain has at least 95%, preferably 100%, sequence identity to SEQ ID NO: 5, and the VL domain has at least 95%, preferably 100%, sequence identity to SEQ ID NO: 7. In some such embodiments, the scFv comprises an amino acid sequence having at least 95%, preferably 100%, sequence identity to SEQ ID NO: 47. In some such embodiments, the scFv comprises an amino acid sequence having at least 95%, preferably 100%, sequence identity to SEQ ID NO: 49. In some such embodiments, the chimeric antigen receptor comprises an amino acid sequence having at least 95%, preferably 100%, sequence identity to SEQ ID NO: 55. In some such embodiments, the chimeric antigen receptor comprises an amino acid sequence having at least 95%, preferably 100%, sequence identity to SEQ ID NO: 57.

In some embodiments, the chimeric antigen receptor further comprises a signal peptide. In some embodiments, the signal peptide comprises an amino acid sequence having at least 80%, at least 85%, at least 90%, at least 95%, or more sequence identity to SEQ ID NO: 33. In some embodiments, the signal peptide comprises an amino acid sequence of SEQ ID NO: 33.

In some embodiments, the polynucleotide is an mRNA. In some embodiments, the polynucleotide is a recombinant DNA construct. In some embodiments, the polynucleotide is comprised by a virus (i.e., a viral vector). In some such embodiments, the virus is an adenovirus (i.e., an adenoviral vector), a lentivirus (i.e., a lentiviral vector), a retrovirus (i.e., a retroviral vector), or an adeno-associated virus (i.e., an AAV vector). In particular embodiments, the virus can be a recombinant AAV (i.e., a recombinant AAV vector). In other embodiments, the polynucleotide is a double-stranded DNA sequence integrated into the genome of a cell.

In another aspect, the invention provides a polynucleotide encoding a chimeric antigen receptor having specificity for CD20, wherein the chimeric antigen receptor comprises: (a) a single chain variable fragment (scFv) having specificity for CD20, wherein the scFv comprises: (i) a heavy chain variable (VH) domain comprising a CDRH1 of SEQ ID NO: 9, a CDRH2 of SEQ ID NO: 10, and a CDRH3 of SEQ ID NO: 11, a polypeptide linker, and a light chain variable (VL) domain comprising a CDRL1 of SEQ ID NO: 12, a CDRL2 of SEQ ID NO: 13, and a CDRL3 of SEQ ID NO: 14; or (ii) a VH domain comprising a CDRH1 of SEQ ID NO: 15, a CDRH2 of SEQ ID NO: 16, and a CDRH3 of SEQ ID NO: 17, a polypeptide linker, and a VL domain comprising a CDRL1 of SEQ ID NO: 18, a CDRL2 of SEQ ID NO: 19, and a CDRL3 of SEQ ID NO: 20; (b) a hinge domain; (c) a transmembrane domain; (d) a co-stimulatory domain having at least 80%, at least 85%, at least 90%, at least 95%, at least 96%, at least 97%, at least 98%, at least 99%, or more sequence identity to SEQ ID NO: 21 or SEQ ID NO: 23; and (e) a signaling domain.

In some embodiments, the chimeric antigen receptor comprises: (a) a single chain variable fragment (scFv) having specificity for CD20, wherein the scFv comprises: (i) a heavy chain variable (VH) domain comprising a CDRH1 of SEQ ID NO: 9, a CDRH2 of SEQ ID NO: 10, and a CDRH3 of SEQ ID NO: 11, a polypeptide linker, and a light chain variable (VL) domain comprising a CDRL1 of SEQ ID NO: 12, a CDRL2 of SEQ ID NO: 13, and a CDRL3 of SEQ ID NO: 14; or (ii) a VH domain comprising a CDRH1 of SEQ ID NO: 15, a CDRH2 of SEQ ID NO: 16, and a CDRH3 of SEQ ID NO: 17, a polypeptide linker, and a VL domain comprising a CDRL1 of SEQ ID NO: 18, a CDRL2 of SEQ ID NO: 19, and a CDRL3 of SEQ ID NO: 20; (b) a hinge domain; (c) a transmembrane domain; (d) a co-stimulatory domain having at least 80%, at least 85%, at least 90%, at least 95%, at least 96%, at least 97%, at least 98%, at least 99%, or 100% sequence identity to SEQ ID NO: 21; and (e) a signaling domain.

In some embodiments, the chimeric antigen receptor comprises: (a) a single chain variable fragment (scFv) having specificity for CD20, wherein the scFv comprises: (i) a heavy chain variable (VH) domain comprising a CDRH1 of SEQ ID NO: 9, a CDRH2 of SEQ ID NO: 10, and a CDRH3 of SEQ ID NO: 11, a polypeptide linker, and a light chain variable (VL) domain comprising a CDRL1 of SEQ ID NO: 12, a CDRL2 of SEQ ID NO: 13, and a CDRL3 of SEQ ID NO: 14; or (ii) a VH domain comprising a CDRH1 of SEQ ID NO: 15, a CDRH2 of SEQ ID NO: 16, and a CDRH3 of SEQ ID NO: 17, a polypeptide linker, and a VL domain comprising a CDRL1 of SEQ ID NO: 18, a CDRL2 of SEQ ID NO: 19, and a CDRL3 of SEQ ID NO: 20; (b) a hinge domain; (c) a transmembrane domain; (d) a co-stimulatory domain having at least 80%, at least 85%, at least 90%, at least 95%, at least 96%, at least 97%, at least 98%, at least 99%, or 100% sequence identity to SEQ ID NO: 23; and (e) a signaling domain.

In some embodiments, the polypeptide linker comprises an amino acid sequence having at least 80%, at least 85%, at least 90%, at least 95%, or more sequence identity to SEQ ID NO: 25 or SEQ ID NO: 71. In some embodiments, the polypeptide linker comprises an amino acid sequence of SEQ ID NO: 25. In some embodiments, the polypeptide linker comprises an amino acid sequence of SEQ ID NO: 71. In some embodiments, the hinge domain comprises an amino acid sequence having at least 80%, at least 85%, at least 90%, at least 95%, or more, sequence identity to SEQ ID NO: 27. In some embodiments, the hinge domain comprises an amino acid sequence of SEQ ID NO: 27. In some embodiments, the transmembrane domain comprises an amino acid sequence having at least 80%, at least 85%, at least 90%, at least 95%, or more, sequence identity to SEQ ID NO: 29. In some embodiments, the transmembrane domain comprises an amino acid sequence of SEQ ID NO: 29. In some embodiments, the signaling domain comprises an amino acid sequence having at least 80%, at least 85%, at least 90%, at least 95%, or more, sequence identity to SEQ ID NO: 31. In some embodiments, the signaling domain comprises an amino acid sequence of SEQ ID NO: 31.

In some embodiments, the VH domain has at least 80%, at least 85%, at least 90%, at least 95%, or more, sequence identity to SEQ ID NO: 1; and the VL domain has at least 80%, at least 85%, at least 90%, at least 95%, or more, sequence identity to SEQ ID NO: 3. In some embodiments, the VH domain comprises SEQ ID NO: 1; and the VL domain comprises SEQ ID NO: 3. In some embodiments, the scFv comprises an amino acid sequence having at least 80%, at least 85%, at least 90%, at least 95%, or more, sequence identity to SEQ ID NO: 35 or SEQ ID NO: 37. In some embodiments, the scFv comprises an amino acid of SEQ ID NO: 35 or SEQ ID NO: 37. In some embodiments, he scFv comprises an amino acid sequence of SEQ ID NO: 35. In some embodiments, he scFv comprises an amino acid sequence of SEQ ID NO: 37. In some embodiments, the co-stimulatory domain comprises an amino acid sequence of SEQ ID NO: 21. In some such embodiments, the chimeric antigen receptor comprises an amino acid sequence having at least 80%, at least 85%, at least 90%, at least 95%, or more, sequence identity to SEQ ID NO: 39 or SEQ ID NO: 41. In some such embodiments, the chimeric antigen receptor comprises an amino acid sequence of SEQ ID NO: 39 or SEQ ID NO: 41. In some such embodiments, the chimeric antigen receptor comprises an amino acid sequence of SEQ ID NO: 39. In some such embodiments, the chimeric antigen receptor comprises an amino acid sequence of SEQ ID NO: 41. In other embodiments, the co-stimulatory domain comprises an amino acid sequence of SEQ ID NO: 23. In some such embodiments, the chimeric antigen receptor comprises an amino acid sequence having at least 80%, at least 85%, at least 90%, at least 95%, or more, sequence identity to SEQ ID NO: 43 or SEQ ID NO: 45. In some such embodiments, the chimeric antigen receptor comprises an amino acid sequence of SEQ ID NO: 43 or SEQ ID NO: 45. In some such embodiments, the chimeric antigen receptor comprises an amino acid sequence of SEQ ID NO: 43. In some such embodiments, the chimeric antigen receptor comprises an amino acid sequence of SEQ ID NO: 45.

In some embodiments, the VH domain has at least 80%, at least 85%, at least 90%, at least 95%, or more, sequence identity to SEQ ID NO: 5; and the VL domain has at least 80%, at least 85%, at least 90%, at least 95%, or more, sequence identity to SEQ ID NO: 7. In some such embodiments, the VH domain comprises SEQ ID NO: 5; and the VL domain comprises SEQ ID NO: 7. In some such embodiments, the scFv comprises an amino acid sequence having at least 80%, at least 85%, at least 90%, at least 95%, or more, sequence identity to SEQ ID NO: 47 or SEQ ID NO: 49. In some embodiments, the scFv comprises an amino acid of SEQ ID NO: 47 or SEQ ID NO: 49. In some embodiments, the co-stimulatory domain comprises an amino acid sequence of SEQ ID NO: 21. In some such embodiments, the chimeric antigen receptor comprises an amino acid sequence having at least 80%, at least 85%, at least 90%, at least 95%, or more, sequence identity to SEQ ID NO: 51 or SEQ ID NO: 53. In some such embodiments, the chimeric antigen receptor comprises an amino acid sequence of SEQ ID NO: 51 or SEQ ID NO: 53. In some such embodiments, the chimeric antigen receptor comprises an amino acid sequence of SEQ ID NO: 51. In some such embodiments, the chimeric antigen receptor comprises an amino acid sequence of SEQ ID NO: 53. In other embodiments, the co-stimulatory domain comprises an amino acid sequence of SEQ ID NO: 23. In some such embodiments, the chimeric antigen receptor comprises an amino acid sequence having at least 80%, at least 85%, at least 90%, at least 95%, or more, sequence identity to SEQ ID NO: 55 or SEQ ID NO: 57. In some such embodiments, the chimeric antigen receptor comprises an amino acid sequence of SEQ ID NO: 55 or SEQ ID NO: 57. In some such embodiments, the chimeric antigen receptor comprises an amino acid sequence of SEQ ID NO: 55. In some such embodiments, the chimeric antigen receptor comprises an amino acid sequence of SEQ ID NO: 57.

In some embodiments, the chimeric antigen receptor comprises: (a) a single chain variable fragment (scFv) having specificity for CD20, wherein the scFv comprises: a heavy chain variable (VH) domain comprising a CDRH1 of SEQ ID NO: 9, a CDRH2 of SEQ ID NO: 10, and a CDRH3 of SEQ ID NO: 11; a polypeptide linker; and a light chain variable (VL) domain comprising a CDRL1 of SEQ ID NO: 12, a CDRL2 of SEQ ID NO: 13, and a CDRL3 of SEQ ID NO: 14; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:21; and (e) a signaling domain comprising an amino acid sequence having at least 95%, preferably 100%, sequence identity to SEQ ID NO:31. In some such embodiments, the VH domain has at least 95%, preferably 100%, sequence identity to SEQ ID NO: 1, and the VL domain has at least 95%, preferably 100%, sequence identity to SEQ ID NO: 3. In some such embodiments, the scFv comprises an amino acid sequence having at least 95%, preferably 100%, sequence identity to SEQ ID NO: 35. In some such embodiments, the scFv comprises an amino acid sequence having at least 95%, preferably 100%, sequence identity to SEQ ID NO: 37. In some such embodiments, the chimeric antigen receptor comprises an amino acid sequence having at least 95%, preferably 100%, sequence identity to SEQ ID NO: 39. In some such embodiments, the chimeric antigen receptor comprises an amino acid sequence having at least 95%, preferably 100%, sequence identity to SEQ ID NO: 41.

In some embodiments, the chimeric antigen receptor comprises: (a) a single chain variable fragment (scFv) having specificity for CD20, wherein the scFv comprises: a heavy chain variable (VH) domain comprising a CDRH1 of SEQ ID NO: 9, a CDRH2 of SEQ ID NO: 10, and a CDRH3 of SEQ ID NO: 11; a polypeptide linker; and a light chain variable (VL) domain comprising a CDRL1 of SEQ ID NO: 12, a CDRL2 of SEQ ID NO: 13, and a CDRL3 of SEQ ID NO: 14; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:23; and (e) a signaling domain comprising an amino acid sequence having at least 95%, preferably 100%, sequence identity to SEQ ID NO:31. In some such embodiments, the VH domain has at least 95%, preferably 100%, sequence identity to SEQ ID NO: 1, and the VL domain has at least 95%, preferably 100%, sequence identity to SEQ ID NO: 3. In some such embodiments, the scFv comprises an amino acid sequence having at least 95%, preferably 100%, sequence identity to SEQ ID NO: 35. In some such embodiments, the scFv comprises an amino acid sequence having at least 95%, preferably 100%, sequence identity to SEQ ID NO: 37. In some such embodiments, the chimeric antigen receptor comprises an amino acid sequence having at least 95%, preferably 100%, sequence identity to SEQ ID NO: 43. In some such embodiments, the chimeric antigen receptor comprises an amino acid sequence having at least 95%, preferably 100%, sequence identity to SEQ ID NO: 45.

In some embodiments, the chimeric antigen receptor comprises: (a) a single chain variable fragment (scFv) having specificity for CD20, wherein the scFv comprises: a heavy chain variable (VH) domain comprising a CDRH1 of SEQ ID NO: 15, a CDRH2 of SEQ ID NO: 16, and a CDRH3 of SEQ ID NO: 17; a polypeptide linker; and a light chain variable (VL) domain comprising a CDRL1 of SEQ ID NO: 18, a CDRL2 of SEQ ID NO: 19, and a CDRL3 of SEQ ID NO: 20; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:21; and (e) a signaling domain comprising an amino acid sequence having at least 95%, preferably 100%, sequence identity to SEQ ID NO:31. In some such embodiments, the VH domain has at least 95%, preferably 100%, sequence identity to SEQ ID NO: 5, and the VL domain has at least 95%, preferably 100%, sequence identity to SEQ ID NO: 7. In some such embodiments, the scFv comprises an amino acid sequence having at least 95%, preferably 100%, sequence identity to SEQ ID NO: 47. In some such embodiments, the scFv comprises an amino acid sequence having at least 95%, preferably 100%, sequence identity to SEQ ID NO: 49. In some such embodiments, the chimeric antigen receptor comprises an amino acid sequence having at least 95%, preferably 100%, sequence identity to SEQ ID NO: 51. In some such embodiments, the chimeric antigen receptor comprises an amino acid sequence having at least 95%, preferably 100%, sequence identity to SEQ ID NO: 53.

In some embodiments, the chimeric antigen receptor comprises: (a) a single chain variable fragment (scFv) having specificity for CD20, wherein the scFv comprises: a heavy chain variable (VH) domain comprising a CDRH1 of SEQ ID NO: 15, a CDRH2 of SEQ ID NO: 16, and a CDRH3 of SEQ ID NO: 17; a polypeptide linker; and a light chain variable (VL) domain comprising a CDRL1 of SEQ ID NO: 18, a CDRL2 of SEQ ID NO: 19, and a CDRL3 of SEQ ID NO: 20; (b) a hinge domain comprising an amino acid sequence having at least 95%, preferably 100%, sequence identity to SEQ ID NO: 27; (c) a transmembrane domain comprising an amino acid sequence having at least 95%, preferably 100%, sequence identity to SEQ ID NO: 29; (d) a co-stimulatory domain having at least 95%, preferably 100%, sequence identity to SEQ ID NO:23; and (e) a signaling domain comprising an amino acid sequence having at least 95%, preferably 100%, sequence identity to SEQ ID NO:31. In some such embodiments, the VH domain has at least 95%, preferably 100%, sequence identity to SEQ ID NO: 5, and the VL domain has at least 95%, preferably 100%, sequence identity to SEQ ID NO: 7. In some such embodiments, the scFv comprises an amino acid sequence having at least 95%, preferably 100%, sequence identity to SEQ ID NO: 47. In some such embodiments, the scFv comprises an amino acid sequence having at least 95%, preferably 100%, sequence identity to SEQ ID NO: 49. In some such embodiments, the chimeric antigen receptor comprises an amino acid sequence having at least 95%, preferably 100%, sequence identity to SEQ ID NO: 55. In some such embodiments, the chimeric antigen receptor comprises an amino acid sequence having at least 95%, preferably 100%, sequence identity to SEQ ID NO: 57.

In some embodiments, the chimeric antigen receptor further comprises a signal peptide. In some embodiments, the signal peptide comprises an amino acid sequence having at least 80%, at least 85%, at least 90%, at least 95%, or more, sequence identity to SEQ ID NO: 33. In some embodiments, the signal peptide comprises an amino acid sequence of SEQ ID NO: 33.

In some embodiments, the polynucleotide is an mRNA. In some embodiments, the polynucleotide is a recombinant DNA construct. In some embodiments, the polynucleotide is comprised by a virus (i.e., a viral vector). In some such embodiments, the virus is an adenovirus (i.e., an adenoviral vector), a lentivirus (i.e., a lentiviral vector), a retrovirus (i.e., a retroviral vector), or an adeno-associated virus (AAV) (i.e., an AAV vector). In particular embodiments, the virus can be a recombinant AAV (i.e., a recombinant AAV vector). In other embodiments, the polynucleotide is a double-stranded DNA sequence integrated into the genome of a cell.

In another aspect, the invention provides a chimeric antigen receptor encoded by any polynucleotide described herein (i.e., a polynucleotide encoding a chimeric antigen receptor).

In another aspect, the invention provides a recombinant DNA construct comprising any polynucleotide described herein (i.e., a polynucleotide encoding a chimeric antigen receptor). In some embodiments, the recombinant DNA construct encodes a virus (i.e., a viral vector) comprising the polynucleotide. In some embodiments, the virus is an adenovirus (i.e., an adenoviral vector), a lentivirus (i.e., a lentiviral vector), a retrovirus (i.e., a retroviral vector), or an adeno-associated virus (AAV) (i.e., an AAV vector). In some embodiments, the virus is a recombinant AAV (i.e., a recombinant AAV vector).

In another aspect, the invention provides a virus (i.e., a viral vector) comprising any polynucleotide described herein (i.e., a polynucleotide encoding a chimeric antigen receptor). In some embodiments, the virus is an adenovirus (i.e., an adenoviral vector), a lentivirus (i.e., a lentiviral vector), a retrovirus (i.e., a retroviral vector), or an adeno-associated virus (i.e., an AAV vector). In some embodiments, the virus is a recombinant AAV (i.e., a recombinant AAV vector).

In another aspect, the invention provides a method of producing a genetically-modified cell, the method comprising introducing into a cell: (a) a first nucleic acid comprising a polynucleotide encoding an engineered nuclease having specificity for a recognition sequence in the genome of the cell, wherein the engineered nuclease is expressed in the cell; and (b) a template nucleic acid comprising any polynucleotide described herein (i.e., a polynucleotide encoding a chimeric antigen receptor); wherein the engineered nuclease generates a cleavage site at the recognition sequence, and wherein the polynucleotide described herein is inserted into the genome at the cleavage site.

In some embodiments, the cell is a T cell, or a cell derived therefrom, and the genetically-modified cell is a genetically-modified T cell, or a cell derived therefrom. In some embodiments, the cell is a human T cell, or a cell derived therefrom, and the genetically-modified cell is a genetically-modified human T cell, or a cell derived therefrom. In some embodiments, the cell is a natural killer (NK) cell, or a cell derived therefrom, and the genetically-modified cell is a genetically-modified NK cell, or a cell derived therefrom. In some embodiments, the cell is a human NK cell and the genetically-modified cell is a genetically-modified human NK cell, or a cell derived therefrom.

In some embodiments, the template nucleic acid is introduced into the cell using a virus (i.e., a viral vector). In some embodiments, the virus is a recombinant AAV (i.e., a recombinant AAV vector).

In some embodiments, the first nucleic acid is an mRNA.

In some embodiments, the recognition sequence is within a target gene, wherein expression of the polypeptide encoded by the target gene is disrupted following insertion of the polynucleotide at the cleavage site. In some embodiments, the target gene is a T cell receptor alpha gene. In some embodiments, the target gene is a T cell receptor alpha constant region gene. In some embodiments, the recognition sequence comprises SEQ ID NO: 66. In some such embodiments, the polynucleotide is inserted into the genome between positions 13 and 14 of SEQ ID NO: 66.

In some embodiments, the engineered nuclease is an engineered meganuclease, a zinc finger nuclease, a TALEN, a compact TALEN, a CRISPR system nuclease, or a megaTAL. In some embodiments, the engineered nuclease is an engineered meganuclease. In some such embodiments, the engineered meganuclease has specificity for a recognition sequence of SEQ ID NO: 66. In particular embodiments, the engineered meganuclease comprises an amino acid sequence of any one of SEQ ID NOs: 68-70.

In another aspect, the invention provides a method of producing a genetically-modified cell, the method comprising introducing into a cell a nucleic acid comprising any polynucleotide described herein (i.e., a polynucleotide encoding a chimeric antigen receptor), wherein the polynucleotide is introduced into the cell by a lentivirus (i.e., a lentiviral vector), and wherein the polynucleotide is randomly integrated into the genome of the cell. In some embodiments, the cell comprises an inactivated T cell receptor alpha gene and/or an inactivated T cell receptor alpha constant region gene. In some embodiments, the cell has no detectable cell surface expression of an endogenous T cell receptor (e.g., an alpha/beta T cell receptor).

In some embodiments, the cell is a T cell, or a cell derived therefrom, and the genetically-modified cell is a genetically-modified T cell, or a cell derived therefrom. In some embodiments, the cell is a human T cell, or a cell derived therefrom, and the genetically-modified cell is a genetically-modified human T cell, or a cell derived therefrom. In some embodiments, the cell is a natural killer (NK) cell, or a cell derived therefrom, and the genetically-modified cell is a genetically-modified NK cell, or a cell derived therefrom. In some embodiments, the cell is a human NK cell and the genetically-modified cell is a genetically-modified human NK cell, or a cell derived therefrom.

In another aspect, the invention provides a genetically-modified cell prepared by any method of producing genetically-modified cells described herein.

In another aspect, the invention provides a genetically-modified cell that expresses a chimeric antigen receptor described herein. In some embodiments, the genetically-modified cell is a genetically-modified T cell, or a cell derived therefrom. In some embodiments, the genetically-modified cell is a genetically-modified human T cell, or a cell derived therefrom. In some embodiments, the genetically-modified cell is a genetically-modified NK cell, or a cell derived therefrom. In some embodiments, the genetically-modified cell is a genetically-modified human NK cell, or a cell derived therefrom.

In another aspect, the invention provides a genetically-modified cell comprising in its genome any polynucleotide described herein (i.e., a polynucleotide encoding a chimeric antigen receptor), wherein the polynucleotide expresses a chimeric antigen receptor and wherein the chimeric antigen receptor is expressed on the cell surface of the genetically-modified cell. In some embodiments, the polynucleotide is inserted into the genome of the genetically-modified cell within a target gene, wherein expression of the polypeptide encoded by the target gene is disrupted. In some embodiments, the target gene is a T cell receptor alpha gene. In some embodiments, the target gene is a T cell receptor alpha constant region gene. In some embodiments, the polynucleotide is inserted into the genome within SEQ ID NO: 66 in the T cell receptor alpha constant region gene. In particular embodiments, the polynucleotide is inserted between positions 13 and 14 of SEQ ID NO: 66 in the T cell receptor alpha constant gene. In some embodiments, the target gene is a T cell receptor alpha constant region gene, and the genetically-modified cell has no detectable cell surface expression of an endogenous T cell receptor.

In some embodiments, the cell is a T cell, or a cell derived therefrom, and the genetically-modified cell is a genetically-modified T cell. In some embodiments, the cell is a human T cell, or a cell derived therefrom, and the genetically-modified cell is a genetically-modified human T cell. In some embodiments, the cell is a natural killer (NK) cell, or a cell derived therefrom, and the genetically-modified cell is a genetically-modified NK cell. In some embodiments, the cell is a human NK cell and the genetically-modified cell is a genetically-modified human NK cell, or a cell derived therefrom.

In another aspect, the invention provides a population of genetically-modified cells comprising a plurality of genetically-modified cells described herein. In some embodiments, at least 30% of cells express the chimeric antigen receptor on their cell surface and have no detectable cell surface expression of an endogenous T cell receptor.

In another aspect, the invention provides a population of cells comprising a plurality of genetically-modified cells described herein. In some embodiments, at least 30% of cells express the chimeric antigen receptor on their cell surface and have no detectable cell surface expression of an endogenous T cell receptor.

In another aspect, the invention provides a pharmaceutical composition comprising a pharmaceutically-acceptable carrier and a population of genetically-modified cells described herein or a population of cells described herein.

In another aspect, the invention provides a pharmaceutical composition comprising a pharmaceutically-acceptable carrier and a genetically-modified cell described herein, wherein the genetically-modified cell comprises a virus (i.e., a viral vector) described herein.

In another aspect, the invention provides a pharmaceutical composition comprising a pharmaceutically-acceptable carrier and a genetically-modified cell described herein, wherein the genetically-modified cell comprises a recombinant DNA construct described herein.

In another aspect, the invention provides a pharmaceutical composition comprising a pharmaceutically-acceptable carrier and a genetically-modified cell described herein, wherein the genetically-modified cell comprises a polynucleotide capable of expressing any chimeric antigen receptor described herein.

In another aspect, the invention provides a pharmaceutical composition comprising a pharmaceutically-acceptable carrier and a genetically-modified cell described herein, wherein the genetically-modified cell comprises any polynucleotide described herein (i.e., a polynucleotide that encodes a chimeric antigen receptor) and expresses any chimeric antigen receptor described herein.

In another aspect, the invention provides a method of immunotherapy for treating cancer in subject in need thereof, the method comprising administering to the subject an effective amount of a genetically-modified cell described herein. In some embodiments, the method comprises administering an effective amount of any pharmaceutical composition described herein which comprises the genetically-modified cells described herein.

In some embodiments, the subject is suffering from a cancer of B-cell origin. In some embodiments, the cancer is selected from the group consisting of B-lineage acute lymphoblastic leukemia, B-cell chronic lymphocytic leukemia and B-cell non-Hodgkin lymphoma. In some embodiments, the cancer is chronic lymphocytic leukemia (CLL) or small lymphocytic lymphoma (SLL). In some embodiments, the cancer is selected from the group consisting of lung cancer, melanoma, breast cancer, prostate cancer, colon cancer, renal cell carcinoma, ovarian cancer, neuroblastoma, rhabdomyosarcoma, leukemia and lymphoma, acute lymphoblastic leukemia, small cell lung cancer, Hodgkin lymphoma, and childhood acute lymphoblastic leukemia. In some embodiments, the pharmaceutical composition is administered in combination with a cancer therapy selected from the group consisting of chemotherapy, surgery, radiation, and gene therapy.

In another aspect, the invention provides a method of treating cancer in subject in need thereof comprising administering to the subject a composition comprising a population of genetically-modified cells described herein, wherein the cells express at least one polynucleotide encoding at least one chimeric antigen receptor described herein. In some embodiments, the genetically-modified cells express one or more polynucleotides encoding at least two chimeric antigen receptors (e.g., express one polynucleotide that encodes two chimeric antigen receptors, or express at least two polynucleotides, each of which encodes one chimeric antigen receptor as described herein). In some embodiments, the at least one chimeric antigen receptor comprises an amino acid sequence selected from the group consisting of SEQ ID NO: 39, SEQ ID NO: 41, SEQ ID NO: 43, SEQ ID NO: 45, SEQ ID NO: 51, SEQ ID NO: 53, SEQ ID NO: 55 and SEQ ID NO: 57. The at least two chimeric antigen receptors may additionally comprise an anti-CD19 chimeric antigen receptor.

In some embodiments, the subject is suffering from a cancer of B-cell origin.

In some embodiments, the cancer is selected from the group consisting of B-lineage acute lymphoblastic leukemia, B-cell chronic lymphocytic leukemia and B-cell non-Hodgkin lymphoma. In some embodiments, the cancer is chronic lymphocytic leukemia (CLL) or small lymphocytic lymphoma (SLL). In some embodiments, the cancer is selected from the group consisting of lung cancer, melanoma, breast cancer, prostate cancer, colon cancer, renal cell carcinoma, ovarian cancer, neuroblastoma, rhabdomyosarcoma, leukemia and lymphoma, acute lymphoblastic leukemia, small cell lung cancer, Hodgkin lymphoma, and childhood acute lymphoblastic leukemia. In some embodiments, the pharmaceutical composition is administered in combination with a cancer therapy selected from the group consisting of chemotherapy, surgery, radiation, and gene therapy.

In another aspect, the invention provides a method for treating cancer in a subject in need thereof, the method comprising administering to the subject genetically-modified cells described herein expressing a chimeric antigen receptor (CAR) that specifically binds to CD20, wherein the CAR comprises: (a) a single chain variable fragment (scFv), wherein the scFv comprises (i) an amino acid sequence having at least 80%, at least 85%, at least 90%, at least 95%, or more, sequence identity to SEQ ID NO: 35 or SEQ ID NO: 37; or (ii) an amino acid sequence having at least 80%, at least 85%, at least 90%, at least 95%, or more, sequence identity to SEQ ID NO: 47 or SEQ ID NO: 49; and (b) a co-stimulatory domain comprising an amino acid sequence having at least 80%, at least 85%, at least 90%, at least 95%, or more, sequence identity to SEQ ID NO: 21 or SEQ ID NO: 23. In some embodiments, the chimeric antigen receptor comprises: (a) an scFv wherein the scFv comprises (i) an amino acid sequence of SEQ ID NO: 35 or SEQ ID NO: 37; or (ii) an amino acid sequence of SEQ ID NO: 47 or SEQ ID NO: 49; and (b) a co-stimulatory domain of comprising an amino acid sequence of SEQ ID NO: 21 or SEQ ID NO: 23. In some embodiments, the chimeric antigen receptor comprises an amino acid sequence selected from the group consisting of SEQ ID NO: 39, SEQ ID NO: 41, SEQ ID NO: 43, SEQ ID NO: 45, SEQ ID NO: 51, SEQ ID NO: 53, SEQ ID NO: 55 and SEQ ID NO: 57.

In some embodiments, the subject is suffering from a cancer of B-cell origin. In certain embodiments, the cancer is selected from the group consisting of B-lineage acute lymphoblastic leukemia, B-cell chronic lymphocytic leukemia and B-cell non-Hodgkin lymphoma. In some embodiments, the cancer is chronic lymphocytic leukemia (CLL) or small lymphocytic lymphoma (SLL). In some embodiments, the cancer is selected from the group consisting of lung cancer, melanoma, breast cancer, prostate cancer, colon cancer, renal cell carcinoma, ovarian cancer, neuroblastoma, rhabdomyosarcoma, leukemia and lymphoma, acute lymphoblastic leukemia, small cell lung cancer, Hodgkin lymphoma, and childhood acute lymphoblastic leukemia.

In some embodiments, the genetically-modified cells are administered in combination with a cancer therapy selected from the group consisting of chemotherapy, surgery, radiation, and gene therapy.

In another aspect, the invention provides a method for reducing the number of cancer cells in a subject, wherein the method comprises administering to the subject an effective amount of a population of genetically-modified cells described herein, a population of cells comprising a plurality of genetically-modified cells described herein, or a pharmaceutical composition described herein comprising genetically-modified cells described herein. In some embodiments, the cancer cells reduced in the subject express CD20 on their cell surface.

In some embodiments, the cancer cells are of B-cell origin. In certain embodiments, the cancer cells are B-lineage acute lymphoblastic leukemia cells, B-cell chronic lymphocytic leukemia cells, or B-cell non-Hodgkin lymphoma cells. In some embodiments, the cancer cells are chronic lymphocytic leukemia (CLL) cells or small lymphocytic lymphoma (SLL) cells. In some embodiments, the cancer cells are selected from the group consisting of lung cancer cells, melanoma cells, breast cancer cells, prostate cancer cells, colon cancer cells, renal cell carcinoma cells, ovarian cancer cells, neuroblastoma cells, rhabdomyosarcoma cells, leukemia and lymphoma cells, acute lymphoblastic leukemia cells, small cell lung cancer cells, Hodgkin lymphoma cells, and childhood acute lymphoblastic leukemia cells.

In another aspect, the invention provides a genetically-modified cell described herein comprising in its genome any polynucleotide described herein (i.e., a polynucleotide encoding a chimeric antigen receptor), wherein the polynucleotide expresses a chimeric antigen receptor and wherein the chimeric antigen receptor is expressed on the cell surface of the genetically-modified cell for use as a medicament. In one embodiment, the invention provides a population of genetically-modified cells described herein, or a population of cells comprising a plurality of genetically-modified cells described herein, for use as a medicament. In another aspect, the invention provides a pharmaceutical composition comprising a pharmaceutically-acceptable carrier and a genetically-modified cell described herein for use as a medicament.

In another aspect, the invention provides a genetically-modified cell described herein comprising in its genome any polynucleotide described herein (i.e., a polynucleotide encoding a chimeric antigen receptor), wherein the polynucleotide expresses a chimeric antigen receptor and wherein the chimeric antigen receptor is expressed on the cell surface of the genetically-modified cell for use in the treatment of cancer in a subject in need thereof. In one embodiment, the invention provides a population of genetically-modified cells described herein, or a population of cells comprising a plurality of genetically-modified cells described herein, for use in the treatment of cancer in a subject in need thereof. In another aspect, the invention provides a pharmaceutical composition comprising a pharmaceutically-acceptable carrier and a genetically-modified cell described herein for use in the treatment of cancer in a subject in need thereof. In some embodiments, the cancer is selected from the group consisting of B-lineage acute lymphoblastic leukemia, B-cell chronic lymphocytic leukemia and B-cell non-Hodgkin lymphoma. In some embodiments, the cancer is chronic lymphocytic leukemia (CLL) or small lymphocytic lymphoma (SLL).

In another aspect, the invention provides the use of a genetically-modified cell described herein comprising in its genome any polynucleotide described herein (i.e., a polynucleotide encoding a chimeric antigen receptor), wherein the polynucleotide expresses a chimeric antigen receptor and wherein the chimeric antigen receptor is expressed on the cell surface of the genetically-modified cell for the manufacture of a medicament for treating cancer. In one embodiment, provided herein is the use of a population of genetically-modified cells described herein, or a population of cells comprising a plurality of genetically-modified cells described herein, for the manufacture of a medicament for the treatment of cancer. In another aspect, the invention provides use of a pharmaceutical composition comprising a pharmaceutically-acceptable carrier and a genetically-modified cell described herein for the manufacture of a medicament for the treatment of cancer. In some embodiments, the cancer is selected from the group consisting of B-lineage acute lymphoblastic leukemia, B-cell chronic lymphocytic leukemia and B-cell non-Hodgkin lymphoma.

Another aspect described herein is a kit comprising a container comprising any polynucleotide described herein with reagents and/or instructions for use.

### BRIEF DESCRIPTION OF THE FIGURES

FIG. 1 provides flow cytometry dot plots depicting the phenotype of chimeric antigen receptor expressing T cells (CAR T) production runs using the 7206 (CD19 scFv CAR), 7260 (muCD20 scFv based CAR), or 7261 (huCD20 scFv based CAR) vectors. The top row of dot plots shows editing and knock-in efficiency for each construct while the bottom row of dot plots shows the CD4:CD8 ratio of the CD3-CAR+ events in the corresponding sample. A) CAR knock in and CD3 knockout frequency for construct 7206. B) CD4:CD8 frequency for construct 7206. C) CAR knock in and CD3 knockout frequency for construct 7260. D) CD4:CD8 frequency for construct 7260. E) CAR knock in and CD3 knockout frequency for construct 7261. F) CD4:CD8 frequency for construct 7261.

FIG. 2 provides flow cytometry dot plots depicting the central memory, transitional memory, and effector memory subsets in the CAR+ population of CART production runs using the 7206 (CD19 scFv CAR), 7260 (muCD20 scFv CAR), or 7261 (huCD20 scFv CAR) vectors. These phenotypes are showing CD62L and CD45RO expression (top row). CD27 high frequencies are displayed in the plots on the bottom row. A) CD45RO and CD62L staining for construct 7206. B) CD27 staining for construct 7260. C) CD45RO and CD62L staining for construct 7206. D) CD27 staining for construct 7260. E) CD45RO and CD62L staining for construct 7261. F) CD27 staining for construct 7261.

FIGS. 3A and 3B provide graphs showing the proliferation of 7260 (FIG. 3A) and 7261 (FIG. 3B) CAR T cells after co-culture with antigen-bearing tumor cells. CAR-T cells were cultured with CD20+ targets at the indicated E:T ratios and at 6d following culture setup, T cells were enumerated. The horizontal line denotes the input T cell number.

FIGS. 4A and 4B provide graphs showing the number of surviving target cells after co-culture of the 7260 or 7261 CAR T cells with CD20+ target cells at varying E:T ratios after 6 days of culture.

FIGS. 5A, 5B, 5C, and 5D provide graphs showing effector IL-2 (FIG. 5A), IFNγ (FIG. 5B), TNFa (FIG. 5C), and granzyme B (FIG. 5D) cytokine levels. The 7260 and/or 7261 CAR T cells were cultured with CD20+ target cells (7260+K20 or 7261+K20), alone (7260 alone or 7261 alone) or with CD20− target cells (7261+K562).

FIG. 6 provides a Kaplan-Meier overall survival of NSG mice injected with Raji lymphoma cells treated with T cell receptor knock out (TCR KO) control cells, vehicle, or the 7260 or 7261 CAR T cells administered at a dosage of either 1e6 cells or 5e6 cells.

FIG. 7 provides a graph showing the overall tumor volume of NSG mice injected with Raji lymphoma cells treated with T cell receptor knock out (TCR KO) control cells, vehicle, or the 7260 or 7261 CAR T cells administered at a dosage of either 1e6 cells or 5e6 cells.

FIG. 8 provides flow cytometry dot plots of the number of CAR+CD3− CAR T cells engineered with four different CAR constructs based on the 7261 CAR construct with the N6 co-stimulatory domain switched for a native 4-1BB co-stimulatory domain (7362), an inactive 4-1BB mutant (7363) co-stimulatory domain, or a novel N1 co-stimulatory domain (7364). A) CAR knock in and CD3 knockout frequency for construct 7261. B) Frequency of CAR+ cells in the CD3− population for construct 7261. C) CAR knock in and CD3 knockout frequency for construct 7362. D) Frequency of CAR+ cells in the CD3− population for construct 7362. E) CAR knock in and CD3 knockout frequency for construct 7363. F) Frequency of CAR+ cells in the CD3− population for construct 7363. G) CAR knock in and CD3 knockout frequency for construct 7364. H) Frequency of CAR+ cells in the CD3-population for construct 7364. Data in the Upper Left quadrant (**) and Lower Left quadrant (++) is indicated in the dot plots for CAR constructs 7261, 7362, 7363, and 7364.

FIG. 9 provides a graph of cell proliferation of the CAR T cells after co-culture with CD20+ target cells. The tested CAR T cells were engineered with four different CAR constructs based on the 7261 CAR construct with the N6 co-stimulatory domain switched for a native 4-1BB co-stimulatory domain (7362), an inactive 4-1BB mutant co-stimulatory domain (7363), or a novel N1 co-stimulatory domain (7364).

FIG. 10 provides a graph of cumulative CD20+ target cells killed when co-cultured with CAR T cells engineered with a CD20 specific CAR. The tested CAR T cells were engineered with four different CAR constructs based on the 7261 CAR construct with the N6 co-stimulatory domain switched for a native 4-1BB co-stimulatory domain (7362), an inactive 4-1BB mutant co-stimulatory domain (7363), or a novel N1 co-stimulatory domain (7364).

FIG. 11 provides flow cytometry data summarizing the percentage of cells that were CD3-AR+ in 3 CD20 CAR T donor batches (CD20Donor1, CD20Donor2, and CD20Donor3) post-depletion of residual unedited CD3+ cells. Anti-CD3 and anti-idiotype antibodies were used to detect gene-edited CD3− T cells that are CAR+ cells. CD3− cell frequencies and CD20 CAR T cell frequencies are displayed in the right-hand panels. A) CD20Donor1 data. B) CD20Donor2 data. C) CD20Donor3 data. The T cells (#), the white blood cells (\hat{ }), the SSC singlets (*), and FSC singlets (+) are indicated in the dot plots.

FIG. 12 provides flow cytometry data summarizing the percentage of CD3-CAR+CD4+ and CD3-CAR+CD8+ cells that are naïve (Tn), central memory (Tcm), and effector memory (Tem) phenotype in 3 CD20 CAR T donor batches (CD20Donor1, CD20Donor2, and CD20Donor3), using anti-CD45RA and anti-CCR7 antibodies. Anti-CD4 and anti-CD8 antibodies were used to detect the CD4+ and CD8+ composition of CD3-CAR+ T cells. A) CD20Donor1 CD4:CD8 data. B) CD20Donor1 CAR+ data in CD4+ cells. C) CD20Donor1 CAR+ data in CD8+ cells. D) CD20Donor2 CD4:CD8 data. E) CD20Donor2 CAR+ data in CD4+ cells. F) CD20Donor2 CAR+ data in CD8+ cells. G) CD20Donor3 CD4:CD8 data. H) CD20Donor3 CAR+ data in CD4+ cells. I) CD20Donor3 CAR+ data in CD8+ cells.

FIG. 13 provides data summarizing proliferative responses of CD20 CAR T cells from 3 donor batches (CD20Donor1, CD20Donor2, and CD20Donor3) following coculture with CD20+ K20 target cells or CD20− K562 target cells. CD20 CAR T cell proliferative responses against the target cells at E:T ratios ranging from 1:1 to 1:9 were measured after 5 days of coculture. The dotted horizontal line represents the input number of CD20 CAR T cells (2×10⁴ cells). A) CD20Donor1 data. B) CD20Donor2 data. C) CD20Donor3 data.

FIG. 14 provides data summarizing cytotoxic responses of CD20 CAR T cells from 3 donor batches (CD20Donor1, CD20Donor2, and CD20Donor3) following coculture with CD20+ K20 target cells or CD20− K562 target cells. CD20 CAR T cells were cocultured at the indicated E:T ratios with CD20+ K20 cells or CD20− K562 cells, and the cytotoxic response of the CD20 CAR T cells was assessed after 5 days of coculture. A) CD20Donor1 data. B) CD20Donor2 data. C) CD20Donor3 data.

FIG. 15 provides data summarizing cytokine secretion by CD20 CAR T cells from 3 donor batches (CD20Donor1, CD20Donor2, and CD20Donor3) following coculture with CD20+ K20 target cells or CD20− K562 target cells. CD20 CAR T cells were cocultured at a ratio of 1:1 with CD20+ K20 cells and CD20− K562 cells for 48 hours in medium in the absence of exogenous cytokines. The secretion of cytokines IFNγ, IL-2, IL-6, and TNFα were measured by ProteinSimple multiplex assay. A) IFNγ secretion. B) IL-2 secretion. C) IL-6 secretion. D) TNF-α secretion.

FIG. 16 provides a Kaplan Meir survival plot following administration of CD20 CAR T cells (CD20Donor2) to NSG mice subcutaneously implanted with Granta-519 cells. NSG mice were implanted with 1×10⁶ Granta-519 cells subcutaneously on the right flank. On Day 1 (16 days postimplantation), animals were given vehicle control, CD3− control T cells, or CD20 CAR T cells via intravenous injection in a lateral tail vein. Cryopreserved CD3− control T cells (5×10⁶) or CD20 CAR T cells were thawed, washed and resuspended in sterile diluent and injected at a dose of 1×10⁶, 5×10⁶, or 1×10⁷ in a total volume of 0.2 mL per animal. Percent survival was plotted for each treatment group.

FIG. 17 provides time to endpoint data following administration of CD20 CAR T cells (CD20Donor2) to NSG mice subcutaneously implanted with Granta-519 cells, as described in FIG. 16. Time to endpoints were plotted for each animal in each group.

FIG. 18 provides data showing tumor volume following administration of CD20 CAR T cells (CD20Donor2) to NSG mice subcutaneously implanted with Granta-519 cells, as described in FIG. 16. Mean tumor volumes were plotted for each treatment group.

### BRIEF DESCRIPTION OF THE SEQUENCES

SEQ ID NO: 1 is the amino acid sequence of a muCD20 heavy chain variable (VH) region.

SEQ ID NO: 2 is the nucleic acid sequence of a muCD20 heavy chain variable (VH) region.

SEQ ID NO: 3 is the amino acid sequence of a muCD20 light chain variable (VL) region.

SEQ ID NO: 4 is the nucleic acid sequence of a muCD20 light chain variable (VL) region.

SEQ ID NO: 5 is the amino acid sequence of a huCD20 heavy chain variable (VH) region.

SEQ ID NO: 6 is the nucleic acid sequence of a huCD20 heavy chain variable (VH) region.

SEQ ID NO: 7 is the amino acid sequence of a huCD20 light chain variable (VL) region.

SEQ ID NO: 8 is the nucleic acid sequence of a huCD20 light chain variable (VL) region.

SEQ ID NO: 9 is the amino acid sequence of the CDRH1 of the muCD20 heavy chain variable (VH) region.

SEQ ID NO: 10 is the amino acid sequence of the CDRH2 of the muCD20 heavy chain variable (VH) region.

SEQ ID NO: 11 is the amino acid sequence of the CDRH3 of the muCD20 heavy chain variable (VH) region.

SEQ ID NO: 12 is the amino acid sequence of the CDRL1 of the muCD20 light chain variable (VL) region.

SEQ ID NO: 13 is the amino acid sequence of the CDRL2 of the muCD20 light chain variable (VL) region.

SEQ ID NO: 14 is the amino acid sequence of the CDRL3 of the muCD20 light chain variable (VL) region.

SEQ ID NO: 15 is the amino acid sequence of the CDRH1 of the huCD20 heavy chain variable (VH) region.

SEQ ID NO: 16 is the amino acid sequence of the CDRH2 of the huCD20 heavy chain variable (VH) region.

SEQ ID NO: 17 is the amino acid sequence of the CDRH3 of the huCD20 heavy chain variable (VH) region.

SEQ ID NO: 18 is the amino acid sequence of the CDRL1 of the huCD20 light chain variable (VL) region.

SEQ ID NO: 19 is the amino acid sequence of the CDRL2 of the huCD20 light chain variable (VL) region.

SEQ ID NO: 20 is the amino acid sequence of the CDRL3 of the huCD20 light chain variable (VL) region.

SEQ ID NO: 21 is the amino acid sequence of the co-stimulatory domain N1.

SEQ ID NO: 22 is the nucleic acid sequence of the co-stimulatory domain N1.

SEQ ID NO: 23 is the amino acid sequence of the co-stimulatory domain N6.

SEQ ID NO: 24 is the nucleic acid sequence of the co-stimulatory domain N6.

SEQ ID NO: 25 is the amino acid sequence of a linker.

SEQ ID NO: 26 is the nucleic acid sequence of a linker.

SEQ ID NO: 27 is the amino acid sequence of a CD8 hinge region.

SEQ ID NO: 28 is the nucleic acid sequence of a CD8 hinge region.

SEQ ID NO: 29 is the amino acid sequence of a CD8 transmembrane domain.

SEQ ID NO: 30 is the nucleic acid sequence of a CD8 transmembrane domain.

SEQ ID NO: 31 is the amino acid sequence of a CD3 zeta domain.

SEQ ID NO: 32 is the nucleic acid sequence of a CD3 zeta domain.

SEQ ID NO: 33 is the amino acid sequence of a CD8 peptide signal.

SEQ ID NO: 34 is the nucleic acid sequence of a CD8 peptide signal.

SEQ ID NO: 35 is the amino acid sequence of the muCD20 scFv (VL-Linker-VH).

SEQ ID NO: 36 is the nucleic acid sequence of the muCD20 scFv (VL-Linker-VH).

SEQ ID NO: 37 is the amino acid sequence of the muCD20 scFv (VH-Linker-VL).

SEQ ID NO: 38 is the nucleic acid sequence of the muCD20 scFv (VH-Linker-VL).

SEQ ID NO: 39 is the amino acid sequence of a muCD20 scFv CAR (VL-Linker-VH-CD8-CD8-N1-CD3).

SEQ ID NO: 40 is the nucleic acid sequence of a muCD20 scFv CAR (VL-Linker-VH-CD8-CD8-N1-CD3).

SEQ ID NO: 41 is the amino acid sequence of a muCD20 scFv CAR (VH-Linker-VL-CD8-CD8-N1-CD3).

SEQ ID NO: 42 is the nucleic acid sequence of a muCD20 scFv CAR (VH-Linker-VL-CD8-CD8-N1-CD3).

SEQ ID NO: 43 is the amino acid sequence of a muCD20 scFv CAR (VL-Linker-VH-CD8-CD8-N6-CD3).

SEQ ID NO: 44 is the nucleic acid sequence of a muCD20 scFv CAR (VL-Linker-VH-CD8-CD8-N6-CD3).

SEQ ID NO: 45 is the amino acid sequence of a muCD20 scFv CAR (VH-Linker-VL-CD8-CD8-N6-CD3).

SEQ ID NO: 46 is the nucleic acid sequence of a muCD20 scFv CAR (VH-Linker-VL-CD8-CD8-N6-CD3).

SEQ ID NO: 47 is the amino acid sequence of the huCD20 scFv (VL-Linker-VH).

SEQ ID NO: 48 is the nucleic acid sequence of the huCD20 scFv (VL-Linker-VH).

SEQ ID NO: 49 is the amino acid sequence of the huCD20 scFv (VH-Linker-VL).

SEQ ID NO: 50 is the nucleic acid sequence of the huCD20 scFv (VH-Linker-VL).

SEQ ID NO: 51 is the amino acid sequence of a huCD20 scFv CAR (VL-Linker-VH-CD8-CD8-N1-CD3).

SEQ ID NO: 52 is the nucleic acid sequence of a huCD20 scFv CAR (VL-Linker-VH-CD8-CD8-N1-CD3).

SEQ ID NO: 53 is the amino acid sequence of a huCD20 scFv CAR (VH-Linker-VL-CD8-CD8-N1-CD3).

SEQ ID NO: 54 is the nucleic acid sequence of a huCD20 scFv CAR (VH-Linker-VL-CD8-CD8-N1-CD3).

SEQ ID NO: 55 is the amino acid sequence of a huCD20 scFv CAR (VL-Linker-VH-CD8-CD8-N6-CD3).

SEQ ID NO: 56 is the nucleic acid sequence of a huCD20 scFv CAR (VL-Linker-VH-CD8-CD8-N6-CD3).

SEQ ID NO: 57 is the amino acid sequence of a huCD20 scFv CAR (VH-Linker-VL-CD8-CD8-N6-CD3).

SEQ ID NO: 58 is the nucleic acid sequence of a huCD20 scFv CAR (VH-Linker-VL-CD8-CD8-N6-CD3).

SEQ ID NO: 59 is the amino acid sequence of a CD8 hinge region.

SEQ ID NO: 60 is the amino acid sequence of a CD28 hinge region.

SEQ ID NO: 61 is the amino acid sequence of a hybrid CD8-CD28 hinge region.

SEQ ID NO: 62 is the amino acid sequence of a CD3 transmembrane domain.

SEQ ID NO: 63 is the amino acid sequence of a CD3 transmembrane domain.

SEQ ID NO: 64 is the amino acid sequence of a CD28 transmembrane domain.

SEQ ID NO: 65 is the amino acid sequence of human CD20.

SEQ ID NO: 66 sets forth the nucleic acid sequence of the sense strand of the TRC 1-2 recognition sequence.

SEQ ID NO: 67 sets forth the nucleic acid sequence of the antisense strand of the TRC 1-2 recognition sequence.

SEQ ID NO: 68 sets forth the amino acid sequence of the TRC 1-2L.1592 meganuclease.

SEQ ID NO: 69 sets forth the amino acid sequence of the TRC 1-2L.1775 meganuclease.

SEQ ID NO: 70 sets forth the amino acid sequence of the TRC 1-2x.87EE meganuclease.

SEQ ID NO: 71 is the amino acid sequence of a linker.

SEQ ID NO: 72 is the nucleic acid sequence of a linker.

SEQ ID NO: 73 is the amino acid sequence of a muCD20 scFv CAR (CD8sp-VL-Linker-VH-CD8-CD8-N6-CD3).

SEQ ID NO: 74 is the nucleic acid sequence of a muCD20 scFv CAR (CD8sp-VL-Linker-VH-CD8-CD8-N6-CD3).

SEQ ID NO: 75 is the amino acid sequence of a huCD20 scFv CAR (CD8sp-VL-Linker-VH-CD8-CD8-N6-CD3).

SEQ ID NO: 76 is the nucleic acid sequence of a huCD20 scFv CAR (CD8sp-VL-Linker-VH-CD8-CD8-N6-CD3).

SEQ ID NO: 77 is the amino acid sequence of a huCD20 scFv CAR (CD8sp-VL-Linker-VH-CD8-CD8-41BB-CD3).

SEQ ID NO: 78 is the nucleic acid sequence of a huCD20 scFv CAR (CD8sp-VL-Linker-VH-CD8-CD8-41BB-CD3).

SEQ ID NO: 79 is the amino acid sequence of a huCD20 scFv CAR (CD8sp-VL-Linker-VH-CD8-CD8-41BBmDel-CD3).

SEQ ID NO: 80 is the nucleic acid sequence of a huCD20 scFv CAR (CD8sp-VL-Linker-VH-CD8-CD8-41BBmDel-CD3).

SEQ ID NO: 81 is the amino acid sequence of a huCD20 scFv CAR (CD8sp-VL-Linker-VH-CD8-CD8-N1-CD3).

SEQ ID NO: 82 is the nucleic acid sequence of a huCD20 scFv CAR (CD8sp-VL-Linker-VH-CD8-CD8-N1-CD3).

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

The term “antibody” as used herein in encompasses various antibody structures, including but not limited to antibodies from animal species (e.g., camelid antibodies, goat antibodies, murine antibodies, rabbit antibodies, and the like), humanized antibodies, monoclonal antibodies, polyclonal antibodies, multispecific antibodies (e.g., bispecific antibodies), nanobodies, monobodies, and antibody fragments so long as they exhibit the desired antigen-binding activity. Other examples of antibodies include, without limitation, a dual-variable immunoglobulin domain, a single-chain Fv molecule (scFv), a single domain antibody (sdAb; e.g., a heavy chain only antibody), a diabody, a triabody, an antibody-like protein scaffold, a Fv fragment, a Fab fragment, a F(ab′)₂ molecule, and a tandem di-scFv.

Further, the term “antibody” includes an immunoglobulin molecule comprising, one or more heavy (H) chains and/or one or more light (L) chains. The chains may be inter-connected by disulfide bonds, as well as multimers thereof (e.g., IgM). Each heavy chain (HC) comprises a heavy chain variable region (or domain) (abbreviated herein as HCVR or VH) and a heavy chain constant region (or domain). The heavy chain constant region comprises three domains, CH1, CH2 and CH3. Each light chain (LC) comprises a light chain variable region (abbreviated herein as LCVR or VL) and a light chain constant region. The light chain constant region comprises one domain (CL1). Each VH and VL is composed of three CDRs and four FRs, arranged from amino-terminus to carboxy-terminus in the following order: FR1, CDR1, FR2, CDR2, 1-R3, CDR3, FR4 Immunoglobulin molecules can be of any type (e.g., IgG, IgE, IgM, IgD, IgA and IgY), class (e.g., IgG1, IgG2, IgG3, IgG4, IgA1 and IgA2) or subclass. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. An “intact” or a “full length” antibody, as used herein, refers to an antibody comprising four polypeptide chains, two heavy (H) chains and two light (L) chains. In one embodiment, an intact antibody is an intact IgG antibody.

An “antibody fragment”, “antigen-binding fragment” or “antigen-binding portion” of an antibody refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv); single domain antibodies (sdAbs), and multispecific antibodies formed from antibody fragments.

As used herein, the term “anti-tumor activity” or “anti-tumor effect” refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the genetically-modified cells of the present disclosure in prevention of the occurrence of tumor in the first place.

As used herein, the term “blastoma” refers to a type of cancer that is caused by malignancies in precursor cells or blasts (immature or embryonic tissue).

As used herein, the term “cancer” should be understood to encompass any neoplastic disease (whether or not invasive or metastatic) which is characterized by abnormal or unregulated cell growth. Invasive or metastatic caners have the potential to spread to other parts of the body. Cancers with unregulated or uncontrolled cell division can cause malignant growth or tumors whereas cancers with slowly dividing cells can cause benign growth or tumors. Examples of cancer include, but are not limited to, breast cancer, prostate cancer, ovarian cancer, cervical cancer, skin cancer, pancreatic cancer, colorectal cancer, renal cancer, liver cancer, brain cancer, lymphoma (e.g., B cell non-Hodgkin lymphoma), chronic lymphocytic leukemia (CLL), small lymphocytic lymphoma (SLL), leukemia (e.g., B-cell chronic lymphocytic leukemia, lymphoblastic leukemia, B-lineage acute lymphoblastic leukemia), lung cancer, and the like.

As used herein, the term “carcinoma” refers to a malignant growth made up of epithelial cells.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons. Reference to CDRH1, CDRH2, and CDRH3 refers to the first, second, and third complementarity determining regions of the heavy chain of the antibody or antibody fragment. Likewise, reference to CDRL1, CDRL2, and CDRL3 refers to the first, second, and third complementarity determining regions of the light chain of the antibody or antibody fragment.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, a “chimeric antigen receptor” or “CAR” refers to an engineered receptor that grafts specificity for an antigen (e.g., CD20) or other ligand or molecule onto an immune effector cell (e.g., a T cell or NK cell). A CAR comprises at least an extracellular ligand-binding domain or moiety, a transmembrane domain, and an intracellular domain, wherein the intracellular domain comprises one or more signaling domains and/or co-stimulatory domains.

An extracellular ligand-binding domain or moiety of a CAR can be, for example, an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In particular examples, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

In some embodiments, the extracellular domain of a CAR comprises an autoantigen (see, Payne et al. (2016) Science, Vol. 353 (6295): 179-184), which is recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and are encompassed by the present disclosure.

The intracellular domain of a CAR can include one or more cytoplasmic signaling domains that transmit an activation signal to the T cell following antigen binding. Such cytoplasmic signaling domains can include, without limitation, a CD3 zeta signaling domain

The intracellular domain of a CAR can also include one or more intracellular co-stimulatory domains that transmit a proliferative and/or cell-survival signal after ligand binding. In some cases, the co-stimulatory domain can comprise one or more TRAF-binding domains. Intracellular co-stimulatory domains can be any of those known in the art and can include, without limitation, those co-stimulatory domains disclosed in WO 2018/067697 (herein incorporated by reference in its entirety) including, for example, Novel 1 (“N1”; SEQ ID NO: 21) and Novel 6 (“N6”; SEQ ID NO: 23). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A CAR further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgGl. In certain examples, the hinge region can be a CD8 alpha domain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 2013/0117869 (which is incorporated by reference in its entirety), including but not limited to MmeI, EndA, End1, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the term “a control” or “a control cell” or a “population of control cells” refers to a cell or population of cells that provides a reference point for measuring changes in genotype or phenotype of a genetically-modified cell or population thereof. A control cell or population of control cells may comprise, for example: (a) a wild-type cell, or population thereof, i.e., of the same genotype as the starting material for the genetic alteration which resulted in the genetically-modified cell; (b) a cell, or population thereof, of the same genotype as the genetically-modified cell but which has been transformed with a null construct (i.e., with a construct which has no known effect on the trait of interest); or, (c) a cell, or population thereof, genetically identical to the genetically-modified cell but which is not exposed to conditions or stimuli or further genetic modifications that would induce expression of altered genotype or phenotype.

As used herein, a “co-stimulatory domain” refers to a polypeptide domain which transmits an intracellular proliferative and/or cell-survival signal upon activation. Activation of a co-stimulatory domain may occur following homodimerization of two co-stimulatory domain polypeptides. Activation may also occur, for example, following activation of a construct comprising the co-stimulatory domain (e.g., a CAR). Generally, a co-stimulatory domain can be derived from a transmembrane co-stimulatory receptor, particularly from an intracellular portion of a co-stimulatory receptor. Non-limiting examples of co-stimulatory domains include, but are not limited to, those co-stimulatory domains described elsewhere herein.

As used herein, a “co-stimulatory signal” refers to an intracellular signal induced by a co-stimulatory domain that promotes cell proliferation, expansion of a cell population in vitro and/or in vivo, promotes cell survival, modulates (e.g., upregulates or downregulates) the secretion of cytokines, and/or modulates the production and/or secretion of other immunomodulatory molecules. In some embodiments, a co-stimulatory signal is induced following homodimerization of two co-stimulatory domain polypeptides. In some embodiments, a co-stimulatory signal is induced following activation of a construct comprising the co-stimulatory domain (e.g. a chimeric antigen receptor).

As used herein, the terms “CRISPR” or “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, “detectable cell-surface expression of an endogenous TCR” refers to the ability to detect one or more components of the TCR complex (e.g., an alpha/beta TCR complex) on the cell surface of a T cell (e.g., a CAR T cell), or a population of T cells (e.g., CAR T cells) described herein, using standard experimental methods. Such methods can include, for example, immunostaining and/or flow cytometry specific for components of the TCR itself, such as a TCR alpha or TCR beta chain, or for components of the assembled cell surface TCR complex, such as CD3. Methods for detecting cell surface expression of an endogenous TCR (e.g., an alpha/beta TCR) on an immune cell include those described in MacLeod et al. (2017) Molecular Therapy 25(4): 949-961.

Similarly, the term “no detectable CD3 on the cell surface” refers to lack of detection of CD3 on the surface of a T cell (e.g., a CAR T cell) described herein, or population of T cells (e.g., CAR T cells) described herein, as detected using standard experimental methods in the art. Methods for detecting cell surface expression of CD3 on an immune cell include those described in MacLeod et al. (2017).

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

The term “effector function” refers to a specialized function of a cell. Effector function of a T cell, for example, may be cytolytic activity or helper activity including the secretion of cytokines. An intracellular signaling domain, such as CD3 zeta, can provide an activation signal to the cell in response to binding of the extracellular domain. As discussed, the activation signal can induce an effector function of the cell such as, for example, cytolytic activity or cytokine secretion.

The term “effective amount” or “therapeutically effective amount” refers to an amount sufficient to effect beneficial or desirable biological and/or clinical results. The amount will vary depending on the therapeutic (e.g., a genetically-modified cell such as a CAR T cell or CAR NK cell) formulation or composition, the disease and its severity, and the age, weight, physical condition and responsiveness of the subject to be treated. In specific embodiments, an effective amount of a cell comprising a CAR described herein or pharmaceutical compositions described herein reduces at least one symptom or the progression of a disease (e.g., cancer). For example, an effective amount of the pharmaceutical compositions or genetically-modified cells described herein reduces the level of proliferation or metastasis of cancer, causes a partial or full response or remission of cancer, or reduces at least one symptom of cancer in a subject.

The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.” For example, in some embodiments, a genetically-modified cell is a T cell, such as a genetically-modified human T cell.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

The term “human antibody”, as used herein, refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of one mammalian species, such as a mouse, have been grafted onto human framework sequences. Additional framework region modifications may be made within the human framework sequences. A “humanized form” of an antibody, e.g., a non-human antibody, refers to an antibody that has undergone humanization.

As used herein, a “human T cell” or “T cell” refers to a T cell isolated from a human donor. In some cases, the human donor is not the subject treated according to the method (i.e., the T cells are allogeneic), but instead a healthy human donor. In some cases, the human donor is the subject treated according to the method. T cells, and cells derived therefrom, can include, for example, isolated T cells that have not been passaged in culture, or T cells that have been passaged and maintained under cell culture conditions without immortalization.

As used herein, the terms “human natural killer cell” or “human NK cell” or “natural killer cell” or “NK cell” refers to a type of cytotoxic lymphocyte critical to the innate immune system. The role NK cells play is analogous to that of cytotoxic T-cells in the vertebrate adaptive immune response. NK cells provide rapid responses to virally infected cells and respond to tumor formation, acting at around 3 days after infection. Human NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, the term “leukemia” refers to malignancies of the hematopoietic organs/systems and is generally characterized by an abnormal proliferation and development of leukocytes and their precursors in the blood and bone marrow.

As used herein, the term “sarcoma” refers to a tumor which is made up of a substance like the embryonic connective tissue and is generally composed of closely packed cells embedded in a fibrillary, heterogeneous, or homogeneous substance.

As used herein, the term “linker” refers to a peptide or a short oligopeptide sequence used to join two subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. In one particular embodiment, a linker may have a length of about 2 to 10 amino acids. In another embodiment, a linker may have a length of about 10 to 80 amino acids. In yet another embodiment, a linker may have a length of more than 80 amino acids. In a particular embodiment, a linker may be arranged between the Fv regions of immunoglobulin heavy chain (H chain) and light chain (L chain) fragments. In another embodiment, a linker may be arranged between the transmembrane domain and the intracellular domain of a CAR. In other embodiments, a linker may be arranged between the scFv and the transmembrane domain of a CAR. In a particular embodiment, a linker may have an amino acid sequence as set forth in SEQ ID NO: 25 or SEQ ID NO: 71. In another embodiment, a linker may have an amino acid sequence as set forth in SEQ ID NO: 25. In another embodiment, a linker may have an amino acid sequence as set forth in SEQ ID NO: 71.

In some embodiments, a linker joins two single chain subunits of an engineered meganuclease described herein. In some such embodiments, a meganuclease linker may include a sequence that substantially comprises glycine and serine. In other such embodiments, a meganuclease linker may include, without limitation, any of those encompassed by U.S. Pat. Nos. 8,445,251, 9,340,777, 9,434,931, and 10,041,053. In further such embodiments, a meganuclease linker may comprise residues 154-195 of SEQ ID NO: 68.

As used herein, the term “lymphoma” refers to a group of blood cell tumors that develop from lymphocytes.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells described herein such that cells can be transfected and maintained at 37° C. without observing deleterious effects on cell viability or significant reductions in meganuclease cleavage activity when measured using the methods described herein.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “megaTAL” refers to a single-chain endonuclease comprising a transcription activator-like effector (TALE) DNA binding domain with an engineered, sequence-specific homing endonuclease.

As used herein, the term “melanoma” refers to a tumor arising from the melanocytic system of the skin and other organs.

As used herein, the term with respect to recombinant proteins, the term “modification” means any insertion, deletion, or substitution of an amino acid residue in the recombinant sequence relative to a reference sequence (e.g., a wild-type or a native sequence).

As used herein, the terms “nuclease” and “endonuclease” refers to enzymes which cleave a phosphodiester bond within a polynucleotide chain.

As used herein, the term “operably linked” is intended to mean a functional linkage between two or more elements. For example, an operable linkage between a nucleic acid sequence encoding a nuclease described herein and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol.266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

Whether a nucleic acid sequence is matched/aligned is determined by results of a BLASTn or FASTDB sequence alignment. This percentage is then subtracted from the percent identity, calculated by the BLASTn program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first proteins corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, a “polycistronic” mRNA refers to a single messenger RNA that comprises two or more coding sequences (i.e., cistrons) and encodes more than one protein. A polycistronic mRNA can comprise any element known in the art to allow for the translation of two or more genes from the same mRNA molecule including, but not limited to, an IRES element, a T2A element, a P2A element, an E2A element, and an F2A element.

As used herein, the term “polynucleotide” or “polynucleotide sequence” refers to a sequence of two or more nucleotides connected by a 5′ to 3′ phosphodiester bond or any variant thereof.

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a nonspecific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a Cpf1 CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

Although the recombinant construct as a whole does not occur in nature, portions of the construct may be found in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “reduces” or “reduced” or “reduced expression” refers to any reduction in the symptoms or severity of a disease or any reduction in the proliferation or number of cancerous cells. In either case, such a reduction may be up to 5%, 10%, 20%, 30%, 40%, 50%, 60%, 70%, 80%, 90%, 95%, or up to 100%. Accordingly, the term “reduced” encompasses both a partial reduction and a complete reduction of a disease state. Further, in some embodiments, the term reduced expression refers to any reduction in the expression of an endogenous T cell receptor (e.g., an alpha/beta T cell receptor) or CD3 at the cell surface of a genetically-modified T cell when compared to a control cell. The term reduced can also refer to a reduction in the percentage of cells in a population of cells that express an endogenous polypeptide (i.e., an endogenous alpha/beta T cell receptor or CD3) at the cell surface when compared to a population of control cells. Such a reduction may be up to 5%, 10%, 20%, 30%, 40%, 50%, 60%, 70%, 80%, 90%, 95%, 96%, 97%, 98%, 99%, or up to 100%. Accordingly, the term “reduced” encompasses both a partial knockdown and a complete knockdown of an endogenous T cell receptor (e.g., an alpha/beta T cell receptor) or CD3.

As used herein, reference to “a heavy chain variable (VH) domain comprising a CDRH1, a CDRH2, and a CDRH3 set forth in” a particular SEQ ID NO, or to “a light chain variable (VL) domain comprising a CDRL1, CDRL2, and a CDRL3 set forth in” a particular SEQ ID NO, is intended to mean that the VH or VL domain comprises the CDRs of the VH or VL domain identified by the particular SEQ ID NO. Such CDRs can be identified, for example, by the definitions of Kabat or Chothia, described elsewhere herein.

As used herein, a “single chain variable fragment (scFv)” means a single chain polypeptide derived from an antibody which retains the ability to bind to an antigen, e.g., CD20. An example of the scFv includes an antibody polypeptide which is formed by a recombinant DNA technique and in which Fv regions of immunoglobulin heavy chain (H chain) and light chain (L chain) fragments are linked via a spacer sequence (or linker sequence). Various methods for preparing a scFv are known, and include methods described in U.S. Pat. No. 4,694,778, Science, vol. 242, pp. 423-442 (1988), Nature, vol. 334, p. 54454 (1989), and Science, vol. 242, pp. 1038-1041 (1988).

As used herein, the term “specifically binds” refers to the ability of a binding protein (e.g., a scFv) to recognize and form a complex with a target molecule (e.g., CD20) rather than to other proteins, and that is relatively stable under physiologic conditions. Specific binding can be characterized by an equilibrium dissociation constant of at least about 1×10⁶M or less (e.g., a smaller equilibrium dissociation constant denotes tighter binding). Methods for determining whether two molecules specifically bind are well known in the art and include, for example, equilibrium dialysis, surface plasmon resonance, and the like. Conversely, as used herein, the term “does not detectably bind” refers to an antibody that does not bind a cell (e.g., a genetically-modified cell) at a level significantly greater than background, e.g., binds to the cell at a level less than 10%, 8%, 6%, 5%, or 1% above background. In some embodiments, the antibody binds to the cell at a level less than 10%, 8%, 6%, 5%, or 1% more than an isotype control antibody. In one example, the binding is detected by Western blotting, flow cytometry, ELISA, antibody panning, and/or Biacore analysis.

As used herein wherein referring a nuclease, the term “specificity” means the ability of a nuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

As used herein, the term “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” or “TRAC” refers to a coding sequence of the T cell receptor alpha gene. The TCR alpha constant region includes the wild-type sequence, and functional variants thereof, identified by NCBI Gene ID NO. 28755.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, S1 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the terms “target site” or “target sequence” refers to a region of the chromosomal DNA of a cell comprising a recognition sequence for a nuclease.

As used herein, the terms “transfected” or “transformed” or “transduced” or “nucleofected” refer to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, the term “treat” or “treatment” means to reduce the frequency or severity of at least one sign or symptom of a disease or disorder (e.g., cancer) experienced by a subject. The terms “treatment” or “treating a subject” can further refer to the administration of a cell (e.g., a T cell) comprising a nucleic acid encoding a CAR in an amount sufficient to treat a disease, e.g., cancer, thereby resulting in either partial or complete destruction or elimination of the cancer. In some aspects, a CAR of the invention, a nucleic acid encoding the same, or a genetically-modified cell or population of genetically-modified cells described herein is administered during treatment in the form of a pharmaceutical composition of the invention.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to a viral vector. Viral vectors can include, without limitation, retroviral vectors (i.e., retroviruses), lentiviral vectors (i.e., lentiviruses), adenoviral vectors (i.e., adenoviruses), and adeno-associated viral vectors (AAV) (i.e., AAV vectors).

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell JG, Barbas CF 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values≥0 and ≤2 if the variable is inherently continuous.

### 2.1 Principle of the Invention

Provided herein are compositions and methods for the treatment of a disease, such as cancer, using a CAR or a genetically-modified cell comprising a CAR. The present invention is based, in part, on the discovery of polynucleotides encoding CARs with superior activity compared to conventional CARs. In some embodiments, a polynucleotide is provided that comprises a nucleic acid sequence encoding a CAR described herein. In some embodiments, the CAR is expressed in a host cell or a genetically-modified cell (e.g., a T cell or NK cell). Accordingly, host cells or genetically-modified cells are provided comprising a novel CAR described herein, as well as methods of making cells comprising the novel CAR.

Further disclosed herein are methods of administering a host cell or a genetically-modified cell comprising a CAR described herein, in order to treat or reduce the symptoms or severity of a disease (e.g., cancer). In some embodiments, administration of a host cell or a genetically-modified cell comprising a CAR described herein treats or reduces the symptoms or severity of diseases, such as cancers, autoimmune disorders, and other conditions which can be targeted by host cells or genetically-modified cells of the present disclosure. Also disclosed herein are methods of immunotherapy for treating cancer in a subject in need thereof comprising administering to the subject a pharmaceutical composition comprising a host cell or a genetically-modified cell described herein and a pharmaceutically acceptable carrier.

### 2.2 Chimeric Antigen Receptors (CARs)

Provided herein are host cells and genetically-modified cells expressing a CAR having specificity for human CD20. Generally, a CAR comprises at least an extracellular domain, a transmembrane domain, and an intracellular domain. The intracellular domain, or cytoplasmic domain, can comprise, for example, at least one co-stimulatory domain and one or more signaling domains. The extracellular domain of a CAR can comprise, for example, a target-specific binding element (e.g., a scFv that specifically binds to CD20) otherwise referred to herein as an extracellular ligand-binding domain (also referred to herein as an antigen-binding domain) or moiety.

The CAR of the present disclosure is engineered to specifically bind to human CD20, an antigen that is expressed on the surface of certain human cancers. The amino acid sequence of human CD20 is provided below:

(NCBI REFERENCE SEQUENCE: NP_690605.1)
(SEQ ID NO: 65)
MTTPRNSVNG TFPAEPMKGP IAMQSGPKPL
FRRMSSLVGP TQSFFMRESK TLGAVQIMNG
LFHIALGGLL MIPAGIYAPI CVTVWYPLWG
GIMYIISGSL LAATEKNSRK CLVKGKMIMN
SLSLFAAISG MILSIMDILN IKISHFLKME
SLNFIRAHTP YINIYNCEPA NPSEKNSPST
QYCYSIQSLF LGILSVMLIF AFFQELVIAG
IVENEWKRTC SRPKSNIVLL SAEEKKEQTI
EIKEEVVGLT ETSSQPKNEE DIEIIPIQEE
EEEETETNFP EPPQDQESSP IENDSSP

The extracellular ligand-binding domain or moiety of a CAR can be, for example, an antibody or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In certain instances, the extracellular ligand-binding domain or moiety of a CAR is in the form of a single-chain variable fragment (scFv) derived from a monoclonal antibody, which provides specificity for a particular epitope or antigen (e.g., CD20). In some embodiments, the scFv is attached via a linker sequence. In some embodiments, the scFv is murine, humanized, or fully human.

The extracellular ligand-binding domain of a CAR can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a CAR can also comprise a naturally-occurring ligand for an antigen of interest, or a fragment of a naturally-occurring ligand which retains the ability to bind the antigen of interest.

In some embodiments of the present invention, a CAR includes an extracellular domain comprising an scFv having a heavy chain variable (VH) domain comprising a CDRH1 of SEQ ID NO: 9, a CDRH2 of SEQ ID NO: 10, and a CDRH3 of SEQ ID NO: 11, a polypeptide linker, and a light chain variable (VL) domain comprising a CDRL1 of SEQ ID NO: 12, a CDRL2 of SEQ ID NO: 13, and a CDRL3 of SEQ ID NO: 14. In another embodiment, a CAR of the present disclosure includes an scFv having a heavy chain variable (VH) domain comprising a CDRH1 of SEQ ID NO: 15, a CDRH2 of SEQ ID NO: 16, and a CDRH3 of SEQ ID NO: 17, a polypeptide linker, and a VL domain comprising a CDRL1 of SEQ ID NO: 18, a CDRL2 of SEQ ID NO: 19, and a CDRL3 of SEQ ID NO: 20.

In other embodiments of the present invention, a CAR includes an extracellular domain comprising an scFv having a heavy chain variable (VH) domain comprising at least 80%, at least 85%, at least 90%, at least 95%, at least 99%, or 100% sequence identity to SEQ ID NO: 1, and a light chain variable (VL) domain comprising at least 80%, at least 85%, at least 90%, at least 95%, at least 99%, or 100% sequence identity to SEQ ID NO: 3. In another embodiment, a CAR of the present disclosure, that may be used in the compositions and methods described herein, includes an extracellular domain comprising a scFv having a heavy chain variable (VH) domain comprising at least 80%, at least 85%, at least 90%, at least 95%, at least 99%, or 100% sequence identity to SEQ ID NO: 5, and a light chain variable (VL) domain comprising at least 80%, at least 85%, at least 90%, at least 95%, at least 99%, or 100% sequence identity to SEQ ID NO: 7.

In further examples of the present invention, a CAR includes an extracellular domain comprising an scFv having a heavy chain variable (VH) domain comprising a CDRH1, a CDRH2, and a CDRH3 set forth in SEQ ID NO: 1, a polypeptide linker, and a light chain variable (VL) domain comprising a CDRL1, a CDRL2, and a CDRL3 set forth in SEQ ID NO: 3. In other examples, a CAR includes an extracellular domain comprising an scFv having a VH domain comprising a CDRH1, a CDRH2, and a CDRH3 set forth in SEQ ID NO: 5, a polypeptide linker, and a VL domain comprising a CDRL1, a CDRL2, and a CDRL3 set forth in SEQ ID NO: 7.

The identification of CDR sequences within a VH or VL domain has been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996). In particular examples, the CDR sequences of the VH and VL domains are identified by the Kabat numbering scheme.

It should be understood that the VH and VL domains of an scFv can be arranged such that the VH domain is the 5′ domain and the VL domain is the 3′ domain, or they can be arranged such that the VL domain is the 5′ domain and the VH domain is the 3′ domain, wherein the domains are separated by a linker.

In some examples, the CAR of the invention can include an scFv comprising an amino acid sequence having at least 80%, at least 85%, at least 90%, at least 95%, or more, sequence identity to SEQ ID NO: 35. In certain examples, the CAR of the invention can include an scFv comprising an amino acid sequence of SEQ ID NO: 35. In some examples, the CAR of the invention can include an scFv comprising an amino acid sequence having at least 80%, at least 85%, at least 90%, at least 95%, or more, sequence identity to SEQ ID NO: 37. In certain examples, the CAR of the invention can include an scFv comprising an amino acid sequence of SEQ ID NO: 37. In some examples, the CAR of the invention can include an scFv comprising an amino acid sequence having at least 80%, at least 85%, at least 90%, at least 95%, or more, sequence identity to SEQ ID NO: 47. In certain examples, the CAR of the invention can include an scFv comprising an amino acid sequence of SEQ ID NO: 47. In some examples, the CAR of the invention can include an scFv comprising an amino acid sequence having at least 80%, at least 85%, at least 90%, at least 95%, or more, sequence identity to SEQ ID NO: 49. In certain examples, the CAR of the invention can include an scFv comprising an amino acid sequence of SEQ ID NO: 49.

In certain examples, the CAR of the invention can include an scFv encoded by a nucleic acid sequence having at least 80%, at least 85%, at least 90%, at least 95%, or more, sequence identity to SEQ ID NO: 36. In some examples, the CAR of the invention can include an scFv encoded by a nucleic acid sequence comprising SEQ ID NO: 36. In certain examples, the CAR of the invention can include an scFv encoded by a nucleic acid sequence having at least 80%, at least 85%, at least 90%, at least 95%, or more, sequence identity to SEQ ID NO: 38. In some examples, the CAR of the invention can include an scFv encoded by a nucleic acid sequence comprising SEQ ID NO: 38. In certain examples, the CAR of the invention can include an scFv encoded by a nucleic acid sequence having at least 80%, at least 85%, at least 90%, at least 95%, or more, sequence identity to SEQ ID NO: 48. In some examples, the CAR of the invention can include an scFv encoded by a nucleic acid sequence comprising SEQ ID NO: 48. In certain examples, the CAR of the invention can include an scFv encoded by a nucleic acid sequence having at least 80%, at least 85%, at least 90%, at least 95%, or more, sequence identity to SEQ ID NO: 50. In some examples, the CAR of the invention can include an scFv encoded by a nucleic acid sequence comprising SEQ ID NO: 50.

A CAR comprises a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. For example, transmembrane domains of particular use in this invention may be derived from TCRα, TCRβ, TCRζ, CD3ζ, CD3ε, CD3γ, CD3δ, CD4, CD5, CD8, CD9, CD16, CD22, CD28, CD32, CD33, CD34, CD37, CD45, CD64, CD80, CD86, CD134, CD137, and CD154. However, any transmembrane domain is contemplated for use herein as long as the domain is capable of anchoring a CAR comprising the extracellular domain to a cell membrane. Transmembrane domains can also be identified using any method known in the art or described herein.

In particular embodiments, the transmembrane domain of the CAR is a CD8 transmembrane domain comprising an amino acid sequence having at least 80%, at least 85%, at least 90%, at least 95%, at least 99%, or 100% sequence identity to SEQ ID NO: 29.

In some embodiments, the transmembrane domain is a CD3 transmembrane domain comprising an amino acid sequence having at least 80%, at least 85%, at least 90%, at least 95%, at least 99%, or 100% sequence identity to SEQ ID NO: 62. In another embodiment, the transmembrane domain is a CD3 zeta transmembrane domain comprising an amino acid sequence having at least 80%, at least 85%, at least 90%, at least 95%, at least 99%, or 100% sequence identity to SEQ ID NO: 63. In some embodiments, the transmembrane domain is a CD8α transmembrane polypeptide, or a variant thereof. In yet another embodiment, the transmembrane domain is a CD28 transmembrane domain comprising an amino acid sequence having at least 80%, at least 85%, at least 90%, at least 95%, at least 99%, or 100% sequence identity to SEQ ID NO: 64.

In some embodiments, a CAR disclosed herein further comprises a hinge region. The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcγRllla receptor or IgGl.

In certain embodiments, the hinge region of the CAR is a CD8 hinge region comprising an amino acid sequence having at least 80%, at least 85%, at least 90%, at least 95%, at least 99%, or 100% sequence identity to SEQ ID NO: 27. In another embodiment, the hinge region is a CD8 hinge region comprising an amino acid sequence having at least 80%, at least 85%, at least 90%, at least 95%, at least 99%, or 100% sequence identity to SEQ ID NO: 59. In some embodiments, the hinge region is a CD28 hinge region comprising an amino acid sequence having at least 80%, at least 85%, at least 90%, at least 95%, at least 99%, or 100% sequence identity to SEQ ID NO: 60, or a variant thereof. In some embodiments, the hinge region is a hybrid CD8-CD28 hinge region comprising an amino acid sequence having at least 80%, at least 85%, at least 90%, at least 95%, at least 99%, or 100% sequence identity to SEQ ID NO: 61.

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular signaling domain can include one or more cytoplasmic signaling domains that transmit an activation signal to the T cell following antigen binding. Such cytoplasmic signaling domains can include, without limitation, a CD3 zeta signaling domain (SEQ ID NO: 31).

The intracellular domain of a CAR can also include one or more intracellular co-stimulatory domains that transmit a proliferative and/or cell-survival signal after ligand binding. In some cases, the co-stimulatory domain can comprise one or more TRAF-binding domains. Intracellular co-stimulatory domains can be any of those known in the art and can include, without limitation, those co-stimulatory domains disclosed in WO 2018/067697 including, for example, Novel 1 (“N1”; SEQ ID NO: 21) and Novel 6 (“N6”; SEQ ID NO: 23). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In particular examples, a CAR described herein comprises an intracellular domain comprising at least one co-stimulatory domain, such as those provided in SEQ ID NOs: 21 and 23, or an active variant thereof. In one embodiment, a CAR described herein comprises an intracellular domain comprising at least one co-stimulatory domain comprising at least 80%, at least 85%, at least 90%, at least 95%, at least 99%, or 100% sequence identity to SEQ ID NO: 21. In another embodiment, a CAR described herein comprises an intracellular domain comprising at least one co-stimulatory domain comprising at least 80%, at least 85%, at least 90%, at least 95%, at least 99%, or 100% sequence identity to SEQ ID NO: 23.

In other embodiments, a CAR described herein may comprise at least two co-stimulatory domains, wherein at least one of the co-stimulatory domains is set forth in SEQ ID NOs: 21 or 23, or an active variant thereof. In yet other embodiments, a CAR described herein comprises an intracellular domain comprising 2, 3, 4 or more co-stimulatory molecules in tandem, wherein at least one of the co-stimulatory domains is set forth in SEQ ID NOs: 21 or 23, or an active variant thereof.

The intracellular domains of a CAR described herein may be linked to each other in a specified or random order. In certain embodiments, the intracellular domain of a CAR described herein may contain short polypeptide linker or spacer regions, between 2 to 30 amino acids in length. In other embodiments, the intracellular domain of a CAR described herein may contain short polypeptide linker or spacer regions, between 2 to 10 amino acids in length. In some embodiments, the linker or spacer regions may include an amino acid sequence that substantially comprises glycine and serine.

In some examples, a CAR of the invention can comprise an amino acid sequence having at least 80%, at least 85%, at least 90%, at least 95%, or more, sequence identity to SEQ ID NO: 39. In some examples, a CAR of the invention can comprise an amino acid sequence of SEQ ID NO: 39. In some examples, a CAR of the invention can comprise an amino acid sequence having at least 80%, at least 85%, at least 90%, at least 95%, or more, sequence identity to SEQ ID NO: 41. In some examples, a CAR of the invention can comprise an amino acid sequence of SEQ ID NO: 41. In some examples, a CAR of the invention can comprise an amino acid sequence having at least 80%, at least 85%, at least 90%, at least 95%, or more, sequence identity to SEQ ID NO: 43. In some examples, a CAR of the invention can comprise an amino acid sequence of SEQ ID NO: 43. In some examples, a CAR of the invention can comprise an amino acid sequence having at least 80%, at least 85%, at least 90%, at least 95%, or more, sequence identity to SEQ ID NO: 45. In some examples, a CAR of the invention can comprise an amino acid sequence of SEQ ID NO: 45. In some examples, a CAR of the invention can comprise an amino acid sequence having at least 80%, at least 85%, at least 90%, at least 95%, or more, sequence identity to SEQ ID NO: 51. In some examples, a CAR of the invention can comprise an amino acid sequence of SEQ ID NO: 51. In some examples, a CAR of the invention can comprise an amino acid sequence having at least 80%, at least 85%, at least 90%, at least 95%, or more, sequence identity to SEQ ID NO: 53. In some examples, a CAR of the invention can comprise an amino acid sequence of SEQ ID NO: 53. In some examples, a CAR of the invention can comprise an amino acid sequence having at least 80%, at least 85%, at least 90%, at least 95%, or more, sequence identity to SEQ ID NO: 55. In some examples, a CAR of the invention can comprise an amino acid sequence of SEQ ID NO: 55. In some examples, a CAR of the invention can comprise an amino acid sequence having at least 80%, at least 85%, at least 90%, at least 95%, or more, sequence identity to SEQ ID NO: 57. In some examples, a CAR of the invention can comprise an amino acid sequence of SEQ ID NO: 57. In some examples, a CAR of the invention can comprise an amino acid sequence having at least 80%, at least 85%, at least 90%, at least 95%, or more, sequence identity to SEQ ID NO: 73. In some examples, a CAR of the invention can comprise an amino acid sequence of SEQ ID NO: 73. In some examples, a CAR of the invention can comprise an amino acid sequence having at least 80%, at least 85%, at least 90%, at least 95%, or more, sequence identity to SEQ ID NO: 75. In some examples, a CAR of the invention can comprise an amino acid sequence of SEQ ID NO: 75. In some examples, a CAR of the invention can comprise an amino acid sequence having at least 80%, at least 85%, at least 90%, at least 95%, or more, sequence identity to SEQ ID NO: 77. In some examples, a CAR of the invention can comprise an amino acid sequence of SEQ ID NO: 77. In some examples, a CAR of the invention can comprise an amino acid sequence having at least 80%, at least 85%, at least 90%, at least 95%, or more, sequence identity to SEQ ID NO: 79. In some examples, a CAR of the invention can comprise an amino acid sequence of SEQ ID NO: 79. In some examples, a CAR of the invention can comprise an amino acid sequence having at least 80%, at least 85%, at least 90%, at least 95%, or more, sequence identity to SEQ ID NO: 81. In some examples, a CAR of the invention can comprise an amino acid sequence of SEQ ID NO: 81.

In certain examples, the CAR of the invention can be encoded by a nucleic acid sequence having at least 80%, at least 85%, at least 90%, at least 95%, or more, sequence identity to SEQ ID NO: 40. In some examples, the CAR of the invention can be encoded by a nucleic acid sequence of SEQ ID NO: 40. In certain examples, the CAR of the invention can be encoded by a nucleic acid sequence having at least 80%, at least 85%, at least 90%, at least 95%, or more, sequence identity to SEQ ID NO: 42. In some examples, the CAR of the invention can be encoded by a nucleic acid sequence of SEQ ID NO: 42. In certain examples, the CAR of the invention can be encoded by a nucleic acid sequence having at least 80%, at least 85%, at least 90%, at least 95%, or more, sequence identity to SEQ ID NO: 44. In some examples, the CAR of the invention can be encoded by a nucleic acid sequence of SEQ ID NO: 44. In certain examples, the CAR of the invention can be encoded by a nucleic acid sequence having at least 80%, at least 85%, at least 90%, at least 95%, or more, sequence identity to SEQ ID NO: 46. In some examples, the CAR of the invention can be encoded by a nucleic acid sequence of SEQ ID NO: 46. In certain examples, the CAR of the invention can be encoded by a nucleic acid sequence having at least 80%, at least 85%, at least 90%, at least 95%, or more, sequence identity to SEQ ID NO: 52. In some examples, the CAR of the invention can be encoded by a nucleic acid sequence of SEQ ID NO: 52. In certain examples, the CAR of the invention can be encoded by a nucleic acid sequence having at least 80%, at least 85%, at least 90%, at least 95%, or more, sequence identity to SEQ ID NO: 54. In some examples, the CAR of the invention can be encoded by a nucleic acid sequence of SEQ ID NO: 54. In certain examples, the CAR of the invention can be encoded by a nucleic acid sequence having at least 80%, at least 85%, at least 90%, at least 95%, or more, sequence identity to SEQ ID NO: 56. In some examples, the CAR of the invention can be encoded by a nucleic acid sequence of SEQ ID NO: 56. In certain examples, the CAR of the invention can be encoded by a nucleic acid sequence having at least 80%, at least 85%, at least 90%, at least 95%, or more, sequence identity to SEQ ID NO: 58. In some examples, the CAR of the invention can be encoded by a nucleic acid sequence of SEQ ID NO: 58. In certain examples, the CAR of the invention can be encoded by a nucleic acid sequence having at least 80%, at least 85%, at least 90%, at least 95%, or more, sequence identity to SEQ ID NO: 74. In some examples, the CAR of the invention can be encoded by a nucleic acid sequence of SEQ ID NO: 74. In certain examples, the CAR of the invention can be encoded by a nucleic acid sequence having at least 80%, at least 85%, at least 90%, at least 95%, or more, sequence identity to SEQ ID NO: 76. In some examples, the CAR of the invention can be encoded by a nucleic acid sequence of SEQ ID NO: 76. In certain examples, the CAR of the invention can be encoded by a nucleic acid sequence having at least 80%, at least 85%, at least 90%, at least 95%, or more, sequence identity to SEQ ID NO: 78. In some examples, the CAR of the invention can be encoded by a nucleic acid sequence of SEQ ID NO: 78. In certain examples, the CAR of the invention can be encoded by a nucleic acid sequence having at least 80%, at least 85%, at least 90%, at least 95%, or more, sequence identity to SEQ ID NO: 80. In some examples, the CAR of the invention can be encoded by a nucleic acid sequence of SEQ ID NO: 80. In certain examples, the CAR of the invention can be encoded by a nucleic acid sequence having at least 80%, at least 85%, at least 90%, at least 95%, or more, sequence identity to SEQ ID NO: 82. In some examples, the CAR of the invention can be encoded by a nucleic acid sequence of SEQ ID NO: 82.

In some embodiments, the chimeric antigen receptors described herein are encoded by a polynucleotide comprising a sequence having 1, 2, 3, 4, 5, 6, 7, 8, 9, 10, 11, 12, 13, 14, 15, 20, 25, 30, 35, 40, 45, 50, or more than 50 nucleotides that differ relative to the sequence as set forth in any one of SEQ ID NOs: 40, 42, 44, 46, 52, 54, 56 and 58. These differences may comprise nucleotides that have been inserted, deleted, or substituted relative to the sequence of any one of SEQ ID NOs: 40, 42, 44, 46, 52, 54, 56 and 58. In some embodiments, the disclosed polynucleotides comprise truncations at the 5′ or 3′ end relative to any one of SEQ ID NOs: 40, 42, 44, 46, 52, 54, 56 and 58. In some embodiments, the disclosed polynucleotides contain stretches of about 50, about 75, about 100, about 125, about 150, about 175, or about 180 nucleotides in common with the sequence of any one of SEQ ID NOs: 40, 42, 44, 46, 52, 54, 56 and 58. In some embodiments, a disclosed polynucleotide that varies in identity of up to 20% relative to (i.e., has at least 80% identity to) any of the sequences of SEQ ID NOs: 40, 42, 44, 46, 52, 54, 56 and 58 encodes a chimeric antigen receptor polypeptide that contains a co-stimulatory domain that has at least 95%, or at least 98%, or up to 100% amino acid sequence identity to either of the sequences of SEQ ID NO: 21 or 23. In some such embodiments, the chimeric antigen receptor polypeptide has at least 95% or at least 98% or up to 100% amino acid sequence identity to any of the amino acid sequences of SEQ ID NO: 39, 41, 43, 45, 51, 53, 55, and 57.

In some embodiments, the chimeric antigen receptors described herein are encoded by a polynucleotide comprising a sequence having 1, 2, 3, 4, 5, 6, 7, 8, 9, 10, 11, 12, 13, 14, 15, 20, 25, 30, 35, 40, 45, 50, or more than 50 nucleotides that differ relative to the sequence as set forth in any one of SEQ ID NOs: 74, 76, 78, 80, and 82. These differences may comprise nucleotides that have been inserted, deleted, or substituted relative to the sequence of any one of SEQ ID NOs: 74, 76, 78, 80, and 82. In some embodiments, the disclosed polynucleotides comprise truncations at the 5′ or 3′ end relative to any one of SEQ ID NOs: 74, 76, 78, 80, and 82. In some embodiments, the disclosed polynucleotides contain stretches of about 50, about 75, about 100, about 125, about 150, about 175, or about 180 nucleotides in common with the sequence of any one of SEQ ID NOs: 74, 76, 78, 80, and 82. In some embodiments, a disclosed polynucleotide that varies in identity of up to 20% relative to (i.e., has at least 80% identity to) any of the sequences of SEQ ID NOs: 74, 76, 78, 80, and 82 encodes a chimeric antigen receptor polypeptide that has at least 95% or at least 98% or up to 100% amino acid sequence identity to any of the amino acid sequences of SEQ ID NO: 73, 75, 77, 79, and 81.

Further, it is to be understood that any of the polynucleotides described herein that encode a CAR can be prepared by a routine method, such as recombinant technology. Methods for preparing a CAR described herein may involve, in some embodiments, the generation of a polynucleotide that encodes a polypeptide comprising each of the domains of the CAR (e.g., at least an extracellular domain, a transmembrane domain, and a intracellular domain).

### 2.3 Methods for Producing Recombinant Viruses (i.e., Viral Vectors)

In some embodiments, the present disclosure provides recombinant AAV vectors for use in the compositions and methods of the present disclosure. Recombinant AAV vectors are typically produced in mammalian cell lines such as HEK-293. Because the viral cap and rep genes are removed from the vector to prevent its self-replication and to make room for the therapeutic gene(s) to be delivered (e.g. the endonuclease gene), it is necessary to provide these in trans in the packaging cell line. In addition, it is necessary to provide the “helper” (e.g. adenoviral) components necessary to support replication (Cots D, Bosch A, Chillon M (2013) Curr. Gene Ther. 13(5): 370-81). Frequently, recombinant AAV vectors are produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient. Accordingly, methods are provided herein for producing recombinant AAV vectors comprising at least one nucleic acid (e.g., a polynucleotide encoding a CAR) described herein.

In some embodiments, genetic transfer is accomplished via lentiviruses (i.e., lentiviral vectors). Lentiviruses, in contrast to other retroviruses, in some contexts may be used for transducing certain non-dividing cells. Non-limiting examples of lentiviruses include those derived from a lentivirus, such as Human Immunodeficiency Virus 1 (HIV-1), HIV-2, an Simian Immunodeficiency Virus (SrV), Human T-lymphotropic virus 1 (HTLV-1), HTLV-2 or equine infection anemia virus (E1AV). For example, lentiviruses have been generated by multiply attenuating the HIV virulence genes, for example, the genes env, vif, vpr, vpu and nef are deleted, making the vector safer for therapeutic purposes. Lentiviruses are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In specific embodiments, lentiviruses are prepared using a plasmid encoding the gag, pol, tat, and rev genes cloned from human immunodeficiency virus (HIV) and a second plasmid encoding the envelope protein from vesicular stomatitis virus (VSV-G) used to pseudotype viral particles. A transfer vector, such as the pCDH-EF1-MCS vector, can be used with a suitable promoter such as the JeT promoter or the EF1 promoter. A CAR described herein can then be inserted downstream of the promoter, followed by an IRES and GFP. All three plasmids can then be transfected into lentivirus cells, such as the Lenti-X-293T cells, and lentivirus can then be harvested, concentrated and screened after a suitable incubation time. Accordingly, methods are provided herein for producing recombinant lentiviruses (i.e., lentiviral vectors) comprising at least one nucleic acid (e.g., a polynucleotide encoding a CAR) described herein. Likewise, methods are provided herein for producing recombinant lentiviruses encoding a CAR described herein.

### 2.4 Genetically-Modified Cells and Populations Thereof

Provided herein are cells that are genetically-modified to express a CAR described herein. In specific embodiments, a genetically-modified cell of the invention comprises a polynucleotide encoding a CAR described herein. In certain embodiments of the present disclosure, a polynucleotide or expression cassette which encodes a CAR described herein is present (i.e., integrated) within the genome of the genetically-modified cell or, alternatively, is not integrated into the genome of the cell. In some embodiments, where the polynucleotide or expression cassette is not integrated into the genome, the polynucleotide or expression cassette is present in the genetically-modified cell in a recombinant DNA construct, in an mRNA, in a viral genome, or in another polynucleotide which is not integrated into the genome of the cell.

Thus, in some examples, genetically-modified cells of the invention can contain a polynucleotide encoding a CAR described herein, positioned within the genome of the cell. In certain embodiments, genetically-modified cells contain a polynucleotide encoding a CAR described herein, positioned within the endogenous T cell receptor alpha gene of the cell. In certain other embodiments, a polynucleotide encoding a CAR described herein is positioned within the endogenous T cell receptor alpha constant region gene, such as within exon 1 of the T cell receptor alpha constant region gene. In particular examples, a polynucleotide encoding a CAR described herein is positioned specifically within SEQ ID NO: 66 (i.e., the TRC 1-2 recognition sequence) within the T cell receptor alpha constant region gene. In further examples, a polynucleotide encoding a CAR described herein is positioned between positions 13 and 14 of SEQ ID NO: 66 (i.e., the TRC 1-2 recognition sequence) within the T cell receptor alpha constant region gene.

The genetically-modified cells comprising a CAR described herein can be, for example, eukaryotic cells. In some such examples, the genetically-modified cells are human cells. In further examples, the genetically-modified cells are immune cells, such as T cells, NK cells, macrophages, monocytes, neutrophils, eosinophils, cytotoxic T lymphocytes, or regulatory T cells. A population of immune cells can be obtained from any source, such as peripheral blood mononuclear cells (PBMCs), cord blood, tissue from site of an infection, ascites, pleural effusion, bone marrow, tissues such as spleen, lymph node, thymus, or tumor tissue. A source suitable for obtaining the type of cell desired would be evident to one of skill in the art. In some embodiments, the population of immune cells is derived from PBMCs. Immune cells useful for the invention may also be derived from pluripotent stem cells (e.g., induced pluripotent stem cells) that have been differentiated into an immune cell.

In some particular embodiments, the genetically-modified cells of the invention are T cells or NK cells, particularly human T cells or human NK cells, or cells derived therefrom. Such cells can be, for example, primary T cells or primary NK cells. In certain embodiments, any number of T cell and NK cell lines available in the art may be used. In some embodiments, T cells and NK cells are obtained from a unit of blood collected from a subject using any number of techniques known to the skilled artisan, such as those described herein above. In one embodiment, cells from the circulating blood of an individual are obtained by apheresis.

Methods of preparing cells capable of expressing a CAR described herein may comprise expanding isolated cells ex vivo. Expanding cells may involve any method that results in an increase in the number of cells capable of expressing a CAR described herein, for example, by allowing the cells to proliferate or stimulating the cells to proliferate. Methods for stimulating expansion of cells will depend on the type of cell used for expression of a CAR and will be evident to one of skill in the art. In some embodiments, the cells expressing a CAR described herein are expanded ex vivo prior to administration to a subject.

Genetically-modified cells comprising a CAR described herein can exhibit increased proliferation when compared to appropriate control cells, or populations of control cells, without a particular co-stimulatory domain described herein (e.g., the co-stimulatory domains as set forth in SEQ ID NOs: 21 and 23). In some embodiments, cells comprising at least one of the co-stimulatory domains described herein further exhibit increased activation and proliferation in vitro or in vivo following stimulation with an appropriate antigen. For example, cells, such as CAR T cells and CAR NK cells, can exhibit increased activation, proliferation, and/or increased cytokine secretion compared to a control cell lacking the co-stimulatory domains described herein. Increased cytokine secretion can include the increased secretion of IFN-γ, IL-2, TNF-α, among others. Methods for measuring cell activation and cytokine production are well known in the art, and some suitable methods are provided in the examples herein.

Also provided herein are genetically-modified cells expressing an inducible regulatory construct. In some embodiments, an inducible regulatory construct is a transmembrane or intracellular construct that is expressed in a cell which provides an inducible co-stimulatory signal to promote cell proliferation, cell survival, and/or cytokine secretion. In some embodiments, an inducible regulatory construct comprises one or more co-stimulatory domains, e.g., those set forth in SEQ ID NOs: 21 or 23 such as those described herein, and/or others that are known in the art, which provide a co-stimulatory signal upon activation. Generally, a co-stimulatory signal can be induced, for example, by homodimerization of two inducible regulatory construct polypeptides. An inducible regulatory construct typically comprises a binding domain which allows for homodimerization following binding of a small molecule, an antibody, or other molecule that allows for homodimerization of two construct polypeptides. Dimerization can initiate the co-stimulatory signal to the cell to promote proliferation, survival, and/or cytokine secretion. In some embodiments, wherein the binding domain binds a small molecule, the binding domain comprises an analogue of FKBP12 (e.g., comprising an F36V substitution) and the small molecule is rimiducid (i.e., API 903). Any binding domains known in the art to be useful in such inducible regulatory constructs, such as CAR T cell safety switches and the like, are contemplated in the present disclosure.

Genetically-modified cells of the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

The present disclosure further provides a population of genetically-modified cells comprising a plurality of genetically-modified cells described herein, which comprise in their genome a polynucleotide encoding a CAR described herein. Thus, in various embodiments of the invention, a population of genetically-modified cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are genetically-modified cells that comprise a CAR described herein. In certain embodiments, a population of genetically-modified cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population express a CAR described herein.

The present invention also provides a population of cells comprising a plurality of genetically-modified cells described herein, which comprise in their genome a polynucleotide encoding a CAR described herein. Thus, in various embodiments of the invention, a population of cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are genetically-modified cells that comprise a polynucleotide encoding a CAR described herein, wherein the CAR is expressed by the genetically-modified cells.

Cells modified by the methods and compositions described herein can express a CAR described herein and further lack expression of an endogenous T cell receptor (e.g., an alpha/beta T cell receptor) due to inactivation of the T cell receptor alpha gene and/or the T cell receptor alpha constant region gene. The T cell receptor alpha chain is required for assembly of the endogenous alpha/beta T cell receptor; therefore, disrupted expression of the T cell receptor alpha chain also disrupts assembly of the endogenous alpha/beta T cell receptor on the cell surface. This further results in a lack of detectable expression of CD3 on the cell surface, because CD3 is also a component of the endogenous alpha/beta T cell receptor.

Thus, the invention further provides a population of cells that express a CAR described herein and do not have detectable cell surface expression of an endogenous T cell receptor (e.g., an alpha/beta T cell receptor). For example, the population can include a plurality of genetically-modified cells of the invention which express a CAR described herein (i.e., are CAR+), and do not have detectable cell surface expression of an endogenous T cell receptor (i.e., are TCR−). In various embodiments of the invention, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified cell described herein that is TCR−/CAR+. In a particular example, the population can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, genetically-modified cells that are TCR−/CAR+.

Further provided is a population of cells comprising a plurality of genetically-modified cells described herein which comprise a polynucleotide encoding a CAR described herein, and which express the CAR (i.e., are CAR+). In some such embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified cell described herein that is CAR+. Also provided is a population of cells comprising a plurality of such genetically-modified cells comprising a polynucleotide encoding a CAR described here (i.e., are CAR+), that also comprise an inactivated T cell receptor alpha gene and/or an inactivated T cell receptor alpha constant region gene (i.e., are TCR−). Such cells do not have detectable cell surface expression of an endogenous T cell receptor (i.e., an alpha/beta T cell receptor). In some such embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are such genetically-modified cells that are TCR−/CAR+.

### 2.5 Methods for Producing Genetically-Modified Cells

The present disclosure provides methods for producing genetically-modified cells (e.g., T cells or NK cells) comprising a CAR described herein. In specific embodiments, methods are provided for modifying a cell to comprise a polynucleotide encoding a CAR described herein. In other aspects of the present disclosure, a polynucleotide or an expression cassette encoding a CAR described herein is integrated into the genome of the cell or, in alternative embodiments, is not integrated into the genome of the cell.

In certain embodiments, the polynucleotide encoding a CAR described herein can be introduced into the genome of a cell by random integration using a lentivirus. Such cells can be further modified to comprise an inactivated T cell receptor alpha gene and/or an inactivated T cell receptor alpha constant region gene, such that the resulting cell expresses the CAR but does not express an endogenous alpha/beta T cell receptor on the cell surface.

In other embodiments, the methods of the invention for producing a genetically-modified cell comprise introducing into the cell a first nucleic acid comprising a polynucleotide encoding an engineered nuclease having specificity for a recognition sequence in the genome of the cell, wherein the engineered nuclease is expressed in the cell. The method further comprises introducing into the cell a template nucleic acid comprising a polynucleotide encoding a CAR described herein. According to the method, the engineered nuclease generates a cleavage site at the recognition sequence, and the polynucleotide is inserted into the genome at said cleavage site. As discussed elsewhere, genetically-modified cells produced by the method can be, for example, genetically-modified T cells or genetically-modified NK cells, particularly genetically-modified human T cells, genetically-modified human NK cells, and cells derived therefrom.

The template nucleic acid can be introduced into the cell by any number of means, such as using a virus (i.e., a viral vector). In particular examples of the method, a virus used to introduce the template nucleic acid is a recombinant AAV (i.e., a recombinant AAV vector). Such recombinant AAVs can comprise the template nucleic acid within a viral capsid. This and other methods for introducing the template nucleic acid are further detailed below.

The first nucleic acid, which encodes the engineered nuclease, can also be introduced by any number of means, such as introduction as an mRNA that is expressed by the cell. This and other methods of introducing the first nucleic acid encoding the engineered nuclease, are further detailed below.

In some examples of this method, the nuclease recognition sequence is within a target gene, and expression of the polypeptide encoded by the target gene is disrupted following insertion of the polynucleotide at the cleavage site. The target gene can be, for example, a gene encoding a component of the alpha/beta T cell receptor, such as the T cell receptor alpha gene or the T cell receptor alpha constant region gene. In particular examples, the target gene is a T cell receptor alpha constant region gene. In such cases, the polynucleotide can be inserted anywhere within the T cell receptor alpha gene or the T cell receptor alpha constant region gene, so long as it is inserted in a manner that allows for expression of the CAR. Further, in certain embodiments of the method, the recognition sequence comprises SEQ ID NO: 66, also referred to as the TRC 1-2 recognition sequence, which is present within the T cell receptor alpha constant region gene. Cleavage of SEQ ID NO: 66 by an engineered meganuclease would be expected to produce a cleavage site between positions 13 and 14 of the recognition sequence. As such, in some examples of the method, the polynucleotide encoding a CAR described herein is inserted into the genome between positions 13 and 14 of SEQ ID NO: 66.

The use of nucleases for disrupting expression of an endogenous TCR gene has been disclosed, including the use of zinc finger nucleases (ZFNs), transcription activator-like effector nucleases (TALENs), megaTALs, and CRISPR systems (e.g., Osborn et al. (2016), Mol. Ther. 24(3): 570-581; Eyquem et al. (2017), Nature 543: 113-117; U.S. Pat. No. 8,956,828; U.S. Publication No. US2014/0301990; U.S. Publication No. US2012/0321667). The specific use of engineered meganucleases for cleaving DNA targets in the human TRAC gene has also been previously disclosed. For example, International Publication No. WO 2014/191527, which disclosed variants of the I-OnuI meganuclease that were engineered to target a recognition sequence within exon 1 of the TCR alpha constant region gene. Moreover, in International Publication Nos. WO 2017/062439 and WO 2017/062451, Applicants disclosed engineered meganucleases which have specificity for recognition sequences in exon 1 of the TCR alpha constant region gene. These included “TRC 1-2 meganucleases” which have specificity for the TRC 1-2 recognition sequence (SEQ ID NO: 66) in exon 1 of the TRAC gene. The '439 and '451 publications also disclosed methods for targeted insertion of a CAR coding sequence or an exogenous TCR coding sequence into a cleavage site in the TCR alpha constant region gene.

Thus, any engineered nuclease can be used for targeted insertion of the polynucleotide encoding a CAR described herein including, for example, an engineered meganuclease, a zinc finger nuclease, a TALEN, a compact TALEN, a CRISPR system nuclease, or a megaTAL.

Zinc-finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. Application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). A CRISPR system comprises two components: (1) a CRISPR nuclease; and (2) a short “guide RNA” comprising a ˜20 nucleotide targeting sequence that directs the nuclease to a location of interest in the genome. The CRISPR system may also comprise a tracrRNA. By expressing multiple guide RNAs in the same cell, each having a different targeting sequence, it is possible to target DNA breaks simultaneously to multiple sites in the genome.

Engineered meganucleases that bind double-stranded DNA at a recognition sequence that is greater than 12 base pairs can be used for the presently disclosed methods. A meganuclease can be an endonuclease that is derived from I-CreI and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker.

Nucleases referred to as megaTALs are single-chain endonucleases comprising a transcription activator-like effector (TALE) DNA binding domain with an engineered, sequence-specific homing endonuclease.

In particular embodiments, the nucleases used to practice the invention are single-chain meganucleases. A single-chain meganuclease comprises an N-terminal subunit and a C-terminal subunit joined by a linker peptide. Each of the two domains recognizes half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. For example, nuclease-mediated insertion using engineered single-chain meganucleases has been disclosed in International Publication Nos. WO 2017/062439 and WO 2017/062451. Nuclease-mediated insertion of the polynucleotide can also be accomplished, for example, using an engineered single-chain meganuclease comprising any one of SEQ ID NOs: 68-70.

In some embodiments, mRNA encoding the engineered nuclease is delivered to the cell because this reduces the likelihood that the gene encoding the engineered nuclease will integrate into the genome of the cell.

The mRNA encoding an engineered nuclease can be produced using methods known in the art such as in vitro transcription. In some embodiments, the mRNA comprises a modified 5′ cap. Such modified 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, Calif.), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element. The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In particular embodiments, nucleoside modifications can include a modification of uridine to pseudouridine, and/or a modification of uridine to N1-methyl pseudouridine.

Purified nuclease proteins can be delivered into cells to cleave genomic DNA, which allows for homologous recombination or non-homologous end-joining at the cleavage site with an exogenous nucleic acid molecule encoding a polypeptide of interest as described herein, by a variety of different mechanisms known in the art, including those further detailed herein.

In another particular embodiment, a nucleic acid encoding an engineered nuclease can be introduced into the cell using a single-stranded DNA template. The single-stranded DNA can further comprise a 5′ and/or a 3′ AAV inverted terminal repeat (ITR) upstream and/or downstream of the sequence encoding the engineered nuclease. In other embodiments, the single-stranded DNA can further comprise a 5′ and/or a 3′ homology arm upstream and/or downstream of the sequence encoding the engineered nuclease.

In other embodiments, genes encoding a nuclease of the invention are introduced into a cell using a linearized DNA template. Such linearized DNA templates can be produced by methods known in the art. For example, a plasmid DNA encoding a nuclease can be digested by one or more restriction enzymes such that the circular plasmid DNA is linearized prior to being introduced into a cell.

Purified engineered nuclease proteins, or nucleic acids encoding engineered nucleases, can be delivered into cells to cleave genomic DNA by a variety of different mechanisms known in the art, including those further detailed herein below.

In some embodiments, the nuclease proteins, or DNA/mRNA encoding the nuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49. In an alternative embodiment, engineered nucleases, or DNA/mRNA encoding nucleases, are coupled covalently or non-covalently to an antibody that recognizes a specific cell-surface receptor expressed on target cells such that the nuclease protein/DNA/mRNA binds to and is internalized by the target cells. Alternatively, engineered nuclease protein/DNA/mRNA can be coupled covalently or non-covalently to the natural ligand (or a portion of the natural ligand) for such a cell-surface receptor. (McCall, et al. (2014) Tissue Barriers. 2(4):e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

In some embodiments, nuclease proteins, or DNA/mRNA encoding nucleases, are encapsulated within biodegradable hydrogels for injection or implantation within the desired region of the liver (e.g., in proximity to hepatic sinusoidal endothelial cells or hematopoietic endothelial cells, or progenitor cells which differentiate into the same). Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, nuclease proteins, or DNA/mRNA encoding nucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33(30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors. In some embodiments, the nuclease proteins or DNA/mRNA encoding the nucleases are encapsulated within liposomes or complexed using cationic lipids (see, e.g., LIPOFECTAMINE™, Life Technologies Corp., Carlsbad, Calif.; Zuris et al. (2015) Nat Biotechnol. 33: 73-80; Mishra et al. (2011) J Drug Deliv. 2011:863734). The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, nuclease proteins, or DNA/mRNA encoding nucleases, are encapsulated within polymeric scaffolds (e.g., PLGA) or complexed using cationic polymers (e.g., PEI, PLL) (Tamboli et al. (2011) Ther Deliv. 2(4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease proteins, or DNA/mRNA encoding nucleases, are combined with amphiphilic molecules that self-assemble into micelles (Tong et al. (2007) J Gene Med. 9(11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease proteins, or DNA/mRNA encoding nucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, nuclease proteins, or DNA/mRNA encoding nucleases, are covalently attached to, or non-covalently associated with, multifunctional polymer conjugates, DNA dendrimers, and polymeric dendrimers (Mastorakos et al. (2015) Nanoscale. 7(9): 3845-56; Cheng et al. (2008) J Pharm Sci. 97(1): 123-43). The dendrimer generation can control the payload capacity and size, and can provide a high payload capacity. Moreover, display of multiple surface groups can be leveraged to improve stability, reduce nonspecific interactions, and enhance cell-specific targeting and drug release.

In some embodiments, genes encoding a nuclease are delivered using a virus (i.e., a viral vector). Such viruses are known in the art and include retroviruses (i.e., retroviral vectors), lentiviruses (i.e., lentiviral vectors), adenoviruses (i.e., adenoviral vectors), and adeno-associated viruses (AAVs) (i.e., AAV vectors) (reviewed in Vannucci, et al. (2013 New Microbial. 36:1-22). Recombinant AAVs useful in the invention can have any serotype that allows for transduction of the virus into a target cell type and expression of the nuclease gene in the target cell. In particular embodiments, recombinant AAVs have a serotype of AAV2 or AAV6. Recombinant AAVs can be single-stranded AAVs. AAVs can also be self-complementary such that they do not require second-strand DNA synthesis in the host cell (McCarty, et al. (2001) Gene Ther. 8:1248-54).

If the nuclease genes are delivered in DNA form (e.g. plasmid) and/or via a virus (e.g. AAV) they must be operably linked to a promoter. In some embodiments, this can be a viral promoter such as endogenous promoters from the viral vector (e.g. the LTR of a lentiviral vector) or the well-known cytomegalovirus- or SV40 virus-early promoters. In a preferred embodiment, nuclease genes are operably linked to a promoter that drives gene expression preferentially in the target cell (e.g., a T cell).

In particular embodiments, an mRNA encoding an engineered nuclease of the invention can be a polycistronic mRNA encoding two or more nucleases that are simultaneously expressed in the cell. A polycistronic mRNA can encode two or more nucleases that target different recognition sequences in the same target gene. Alternatively, a polycistronic mRNA can encode at least one nuclease described herein and at least one additional nuclease targeting a separate recognition sequence positioned in the same gene, or targeting a second recognition sequence positioned in a second gene such that cleavage sites are produced in both genes. A polycistronic mRNA can comprise any element known in the art to allow for the translation of two or more genes (i.e., cistrons) from the same mRNA molecule including, but not limited to, an IRES element, a T2A element, a P2A element, an E2A element, and an F2A element.

The invention further provides for the introduction of a template nucleic acid comprising a polynucleotide described herein (i.e., encoding a CAR described herein), wherein the polynucleotide is inserted into a cleavage site in the targeted gene. In some embodiments, the template nucleic acid comprises a 5′ homology arm and a 3′ homology arm flanking the polynucleotide and elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

The polynucleotide encoding the CAR can further comprise additional control sequences. For example, the sequence can include homologous recombination enhancer sequences, Kozak sequences, polyadenylation sequences, transcriptional termination sequences, selectable marker sequences (e.g., antibiotic resistance genes), origins of replication, and the like. Sequences encoding engineered nucleases can also include at least one nuclear localization signal. Examples of nuclear localization signals are known in the art (see, e.g., Lange et al., J. Biol. Chem., 2007, 282:5101-5105).

A template nucleic acid, comprising a polynucleotide described herein (i.e., a polynucleotide encoding a CAR described here), can be introduced into the cell by any of the means previously discussed. In a particular embodiment, the template nucleic acid is introduced by way of a virus, such as a recombinant AAV. Recombinant AAVs useful for introducing a template nucleic acid can have any serotype that allows for transduction of the virus into the cell and insertion of the polynucleotide into the cell genome. In particular embodiments, the recombinant AAV has a serotype of AAV2 or AAV6. Recombinant AAVs can be single-stranded AAV vectors. Recombinant AAVs can also be self-complementary such that they do not require second-strand DNA synthesis in the host cell (McCarty, et al. (2001) Gene Ther. 8: 1248-54).

In another alternative, the template nucleic acid can be introduced into the cell using a single-stranded DNA template. The single-stranded DNA can comprise the polynucleotide and, in preferred embodiments, can comprise 5′ and 3′ homology arms to promote insertion of the polynucleotide into the cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, the template nucleic acid can be introduced into the cell by transfection with a linearized DNA template. In some examples, a plasmid DNA can be digested by one or more restriction enzymes such that the circular plasmid DNA is linearized prior to transfection into the cell.

In particular embodiments, introducing a polynucleotide encoding a CAR described herein into a cell can increase activation, proliferation, and/or cytokine secretion of the cell when compared to a control cell encoding a different CAR lacking a co-stimulatory domain set forth in SEQ ID NOs: 21 or 23.

In particular embodiments, the period of cell proliferation and/or expansion of the cell population, and/or delay cell exhaustion, is prolonged following introduction of a polynucleotide described herein (i.e., a polynucleotide encoding a CAR described herein) when compared to control cells. Methods of measuring cell expansion and exhaustion (such as T cell or NK cell expansion and exhaustion) are known in the art and disclosed elsewhere herein.

T cells modified by the present invention may require activation prior to introduction of a nuclease and/or an exogenous sequence of interest. For example, T cells can be contacted with anti-CD3 and anti-CD28 antibodies that are soluble or conjugated to a support (i.e., beads) for a period of time sufficient to activate the cells.

### 2.6 Pharmaceutical Compositions

In one aspect of the invention, the present disclosure provides a pharmaceutical composition comprising a genetically-modified cell described herein, a population of genetically-modified cells described herein, or a population of cells described herein, and a pharmaceutically-acceptable carrier. Such pharmaceutical compositions can be prepared in accordance with known techniques. See, e.g., Remington, The Science and Practice of Pharmacy (21^{st} ed. 2005). In the manufacture of a pharmaceutical formulation, according to the present disclosure, cells are typically admixed with a pharmaceutically acceptable carrier and the resulting composition is administered to a subject (e.g., a human). The pharmaceutically acceptable carrier must, of course, be acceptable in the sense of being compatible with any other ingredients in the formulation and must not be deleterious to the subject. In some embodiments, the pharmaceutical compositions of the present disclosure further comprise one or more additional agents useful in the treatment of a disease (e.g., cancer) in a subject. In additional embodiments, where the genetically-modified cell is a genetically-modified human T cell or NK cell (or a cell derived therefrom), pharmaceutical compositions of the present disclosure can further include biological molecules, such as cytokines (e.g., IL-2, IL-7, IL-15, and/or IL-21), which promote in vivo cell proliferation and engraftment. Pharmaceutical compositions comprising genetically-modified cells of the present disclosure can be administered in the same composition as an additional agent or biological molecule or, alternatively, can be co-administered in separate compositions.

The present disclosure also provides genetically-modified cells, or populations thereof, described herein for use as a medicament. The present disclosure further provides the use of genetically-modified cells, or populations thereof, described herein in the manufacture of a medicament for treating a disease in a subject in need thereof. In one such aspect, the medicament is useful for cancer immunotherapy in subjects in need thereof.

In some embodiments, the pharmaceutical compositions and medicaments of the present disclosure are useful for treating any disease state that can be targeted by adoptive immunotherapy. In a particular embodiment, the pharmaceutical compositions and medicaments of the present disclosure are useful as immunotherapy in the treatment of cancer. In some embodiments, the pharmaceutical composition is useful for treating a CD20 related disease by killing a CD20 expressing (positive) target cell. In particular examples, the pharmaceutical composition is useful for treating a cancer of B cell origin that expresses CD20. In certain examples, the cancer is B-lineage acute lymphoblastic leukemia, B-cell chronic lymphocytic leukemia and B-cell non-Hodgkin lymphoma. In some examples, the cancer is chronic lymphocytic leukemia (CLL) or small lymphocytic lymphoma (SLL). In other examples, the cancer may be lung cancer, melanoma, breast cancer, prostate cancer, colon cancer, renal cell carcinoma, ovarian cancer, neuroblastoma, rhabdomyosarcoma, leukemia and lymphoma, acute lymphoblastic leukemia, small cell lung cancer, Hodgkin lymphoma, or childhood acute lymphoblastic leukemia, so long as the cancer cells express CD20.

### 2.7 Methods of Administering Genetically-Modified Cells

In another aspect of the invention, a genetically-modified cell described herein, a population of genetically-modified cells described herein, a population of cells described herein, or a pharmaceutical composition described herein, is administered to a subject in need thereof.

For example, an effective amount of such genetically-modified cells, populations, or pharmaceutical compositions can be administered to a subject having a disease or disorder. The genetically-modified cells administered to the subject, which express a CAR described herein, facilitate the reduction of the proliferation, reduce the number, or kill target cells in the recipient. Unlike antibody therapies, genetically-modified cells of the present disclosure are able to replicate and expand in vivo, resulting in long-term persistence that can lead to sustained control of a disease.

Examples of possible routes of administration include parenteral, (e.g., intravenous (IV), intramuscular (IM), intradermal, subcutaneous (SC), or infusion) administration. Moreover, the administration may be by continuous infusion or by single or multiple boluses. In specific embodiments, the agent is infused over a period of less than about 12 hours, less than about 10 hours, less than about 8 hours, less than about 6 hours, less than about 4 hours, less than about 3 hours, less than about 2 hours, or less than about 1 hour. In still other embodiments, the infusion occurs slowly at first and then is increased over time.

In some of these embodiments wherein cancer is treated with the presently disclosed genetically-modified cells, the subject administered the genetically-modified cells is further administered an additional therapeutic agent or treatment, including, but not limited to gene therapy, radiation, surgery, or a chemotherapeutic agent(s) (i.e., chemotherapy).

When an “effective amount” or “therapeutic amount” is indicated, the precise amount of the compositions of the present disclosure to be administered can be determined by a physician with consideration of individual differences in age, weight, tumor size (if present), extent of infection or metastasis, and condition of the patient (subject). In some embodiments, a pharmaceutical composition comprising the genetically-modified cells described herein is administered at a dosage of 10⁴ to 10⁹ cells/kg body weight, including all integer values within those ranges. In further embodiments, the dosage is 10⁵ to 10⁷ cells/kg body weight, including all integer values within those ranges. In some embodiments, cell compositions are administered multiple times at these dosages. The genetically-modified cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

In some embodiments, the administration of genetically-modified cells of the present disclosure reduces at least one symptom of a target disease or condition. For example, administration of genetically-modified cells of the present disclosure can reduce at least one symptom of a cancer, such as cancers of B-cell origin. Symptoms of cancers, such as cancers of B-cell origin, are well known in the art and can be determined by known techniques.

### 2.8 Variants

The present invention encompasses variants of the polypeptide and polynucleotide sequences described herein. As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein. Such variants may result, for example, from human manipulation. Biologically active variants of polypeptides described herein will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, as determined by sequence alignment programs and parameters described elsewhere herein. A biologically active variant of a polypeptide may differ from that polypeptide or subunit by as few as about 1-40 amino acid residues, as few as about 1-20, as few as about 1-10, as few as about 5, as few as 4, 3, 2, or even 1 amino acid residue.

The polypeptides may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a polypeptide or RNA. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide (e.g., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its biological activity.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Design of CD20 CARs and Construction of AAVs

To build anti-CD20 CARs, single-chain variable fragments (scFvs) were designed using the variable heavy (VH) and variable light (VL) chain sequences of two anti-CD20 antibodies. The first antibody is a fully human IgG antibody with specificity against CD20 and is referred to herein as huCD20. The second antibody is a murine antibody with specificity against CD20 and is referred to herein as muCD20. The variable regions from the heavy and light chains for each antibody were cloned and joined by a GS (glycine-serine) linker to form the scFv. The scFv comprising the variable regions of the muCD20 antibody comprised a linker set forth in SEQ ID NO: 71, whereas the scFv comprising the variable regions of the huCD20 antibody comprised a linker set forth in SEQ ID NO: 25. To construct a CAR, the scFv was joined to a CD8 hinge (SEQ ID NO: 27) and CD8 transmembrane (SEQ ID NO: 29) region and an intracellular signaling domain comprising an N6 co-stimulatory domain (SEQ ID NO: 23) and a CD3ζ intracellular signaling domain (SEQ ID NO: 31). In some experiments, the N6 co-stimulatory domain was replaced with an N1 (SEQ ID NO: 21), 4-1BB, or 4-1BB mutant co-stimulatory domain. The CAR further included a CD8 signal peptide (SEQ ID NO: 33). When these CAR molecules interact with CD20+ target cells, the receptors cluster together in the cytoplasmic membrane and transduce signals through the N6-CD3ζ tails.

The CAR constructs described above were placed under the control of a JeT promoter (a synthetic promoter containing four SP1 sites). The following studies utilize a nuclease-mediated targeted insertion approach to produce CD20 CAR T cells. The target insertion site is an engineered meganuclease recognition sequence in the T cell receptor alpha constant region (TRAC) gene, referred to as TRC 1-2 (SEQ ID NO: 66). For preparation of an AAV for delivery, region of homology to the sequences flanking the TRC 1-2 recognition sequence were added to each end of the CAR construct to enable homology-driven insertion into edited TRAC alleles. This construct was then cloned into an AAV6 packaging plasmid and used to transfect packaging cells along with RepCap and a helper plasmid for AAV6 particle production. The design of the CAR constructs tested herein are provided in Table 1.

TABLE 1
CAR Construct Design

| | Signal | | | Trans- | Co- | Intracellular |
|---|---|---|---|---|---|---|
| Construct# | Peptide | scFv | Hinge | membrane | stimulatory | signaling |
| 7260 (SEQ | CD8 | muCD20 | CD8 | CD8 | N6 | CD3 ξ |
| ID NO: 74) | | | | | | |
| 7261 (SEQ | CD8 | huCD20 | CD8 | CD8 | N6 | CD3 ξ |
| ID NO: 76) | | | | | | |
| 7362 (SEQ | CD8 | huCD20 | CD8 | CD8 | 4-1BB | CD3 ξ |
| ID NO: 78) | | | | | | |
| 7363 (SEQ | CD8 | huCD20 | CD8 | CD8 | 4-1BB Del | CD3 ξ |
| ID NO: 80) | | | | | | |
| 7364 (SEQ | CD8 | huCD20 | CD8 | CD8 | N1 | CD3 ξ |
| ID NO: 82) | | | | | | |

### Example 2

### Production and Characterization of CD20-N6 CAR T Cells

### 1. Methods

In this study, an apheresis sample was drawn from a healthy, informed, and compensated donor, and the T cells were enriched using the CD3 positive selection kit II in accord with the manufacturer's instructions (Stem Cell Technologies). T cells were activated using ImmunoCult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 μg of RNA encoding the TRC 1-2L.1592 meganuclease (SEQ ID NO: 68), which recognizes and cleaves the TRC 1-2 recognition sequence in the T cell receptor alpha constant locus, and were transduced with AAV packaged with construct 7260 or 7261 at an MOI of 25000 viral genomes/cell. AAV6-7206 (encoding an anti-CD19 FMC63 CAR) was included as a control. Cultures were carried out for 5 days in complete X-VIVO-15 medium supplemented with 30 ng/ml IL-2 prior to conducting a flow cytometric analysis of CD3 (clone UCHT1, BD Biosciences) and CAR expression to determine the frequency of TRAC knock-out and CAR knock-in cells. To detect CAR expression, two anti-idiotype clones (VM57 anti-muCD20 and VM4 anti-huCD20) were produced and conjugated to AlexaFluor647 in-house. In addition, the frequencies of CD4 and CD8 cells were determined using anti-CD4 clone OKT4, (BD Biosciences) and anti-CD8 clone HIT8a (BioLegend). A panel of surface markers were also measured to assess the degree to which the CAR T cells have differentiated in culture. Specifically, CD62L (clone SK11, BD), CD45RO (clone UCHL1 BioLegend), and CD27 (Clone M-T271 BD) levels were measured. The following phenotypes were used to define the various populations:

CD62L^{HI}CD45RO^{Lo}=Central memory (CM)
CD62L^{HI}CD45RO^{HI}=Transitional memory (TM)
CD62L⁻CD45RO^{HI}=Effector memory (EM)

Positive CD27 expression was also used as an indicator of a central memory phenotype and to set the threshold of CD45RO Lo versus Hi expression (not shown).

### 2. Results

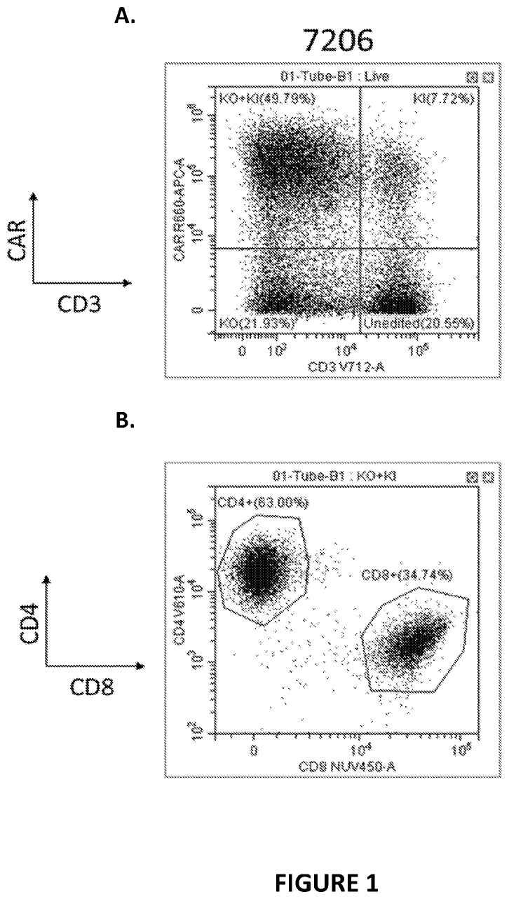

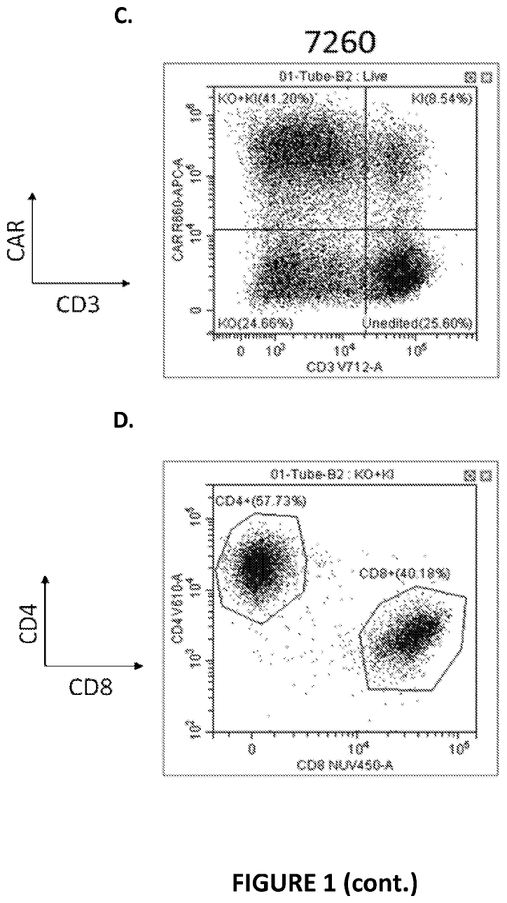

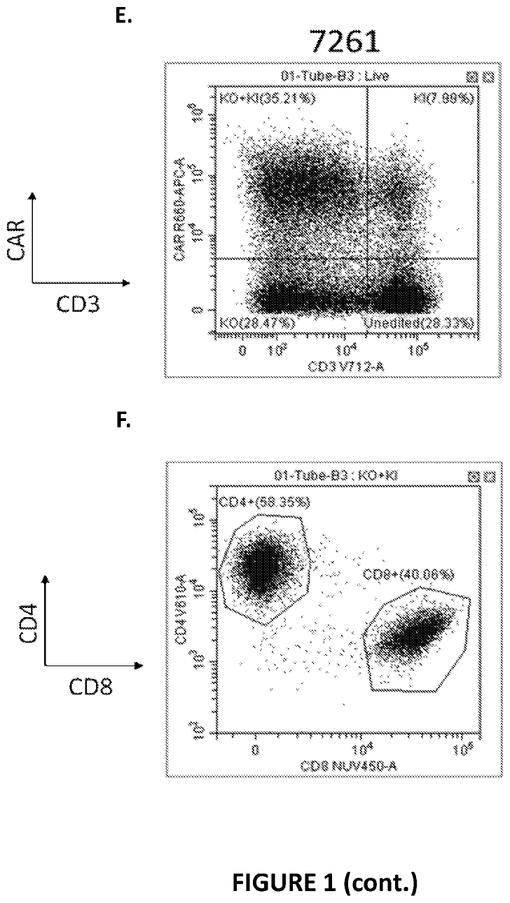

The knock-in/knock-out frequencies of the various CAR T cultures are shown in the CD3 versus CAR dot plots in FIGS. 1A, 1C, and 1E. The overall frequencies of TRAC-edited CAR T cells were found to be 41% and 35% of total cells for the 7260 and 7261 constructs, respectively. By comparison, the analogous population in the 7206 culture was approximately 50%. The CD4:CD8 ratios (FIGS. 1B, 1D, and 1F) from each population of CD3− CAR+ cells were approximately equal, ranging from 1.4-1.8.

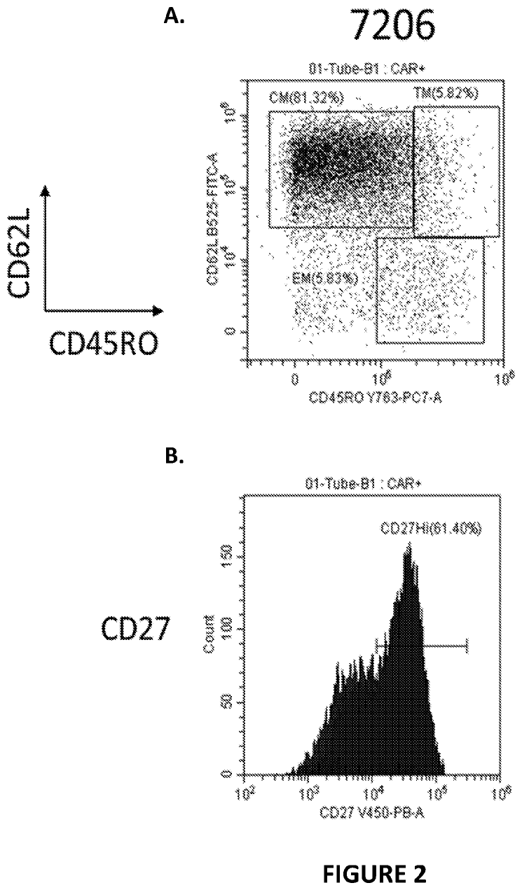

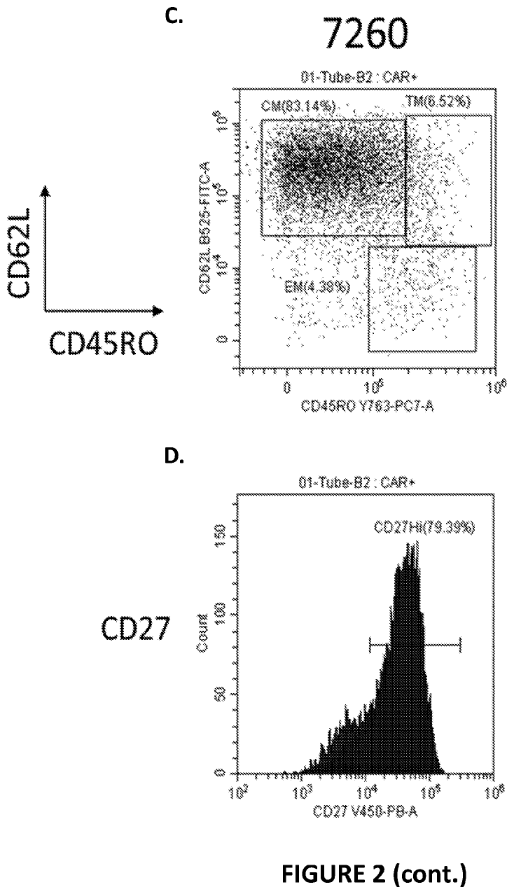

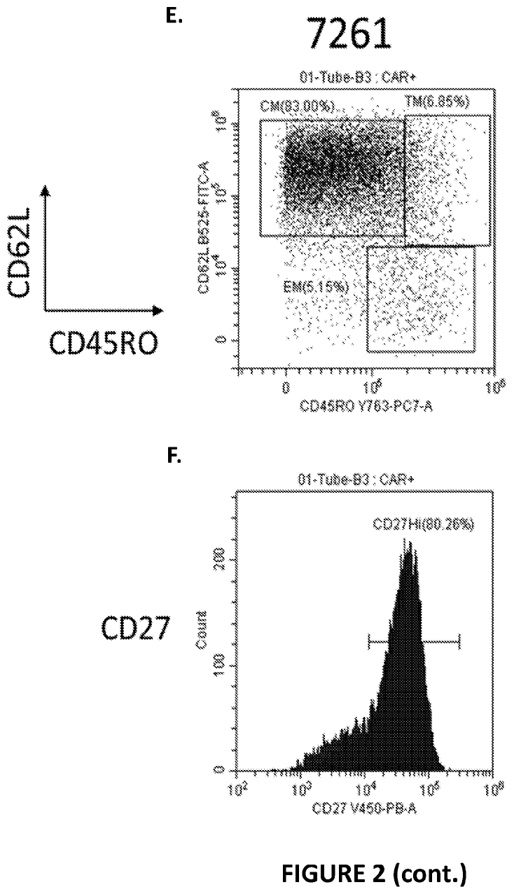

The vast majority (approximately 80%) of CD3− CAR+ cells in the cultures displayed a central memory phenotype (CD62L^{HI}CD45RO^{Lo}), with few cells displaying a more differentiated (TM or EM) phenotype (FIGS. 2A, 2C, and 2E). Frequencies of CD27 cells were likewise between 60-80% (FIGS. 2B, 2D, and 2F).

### 3. Conclusions

Expanding CAR T cells in culture during production carries a risk of differentiating the cells into short-lived populations and a risk of skewing the CD4:CD8 ratio in favor of CD8 T cells. These studies demonstrate the production of anti-CD20 CAR T cells using a murine (7260) or human (7261) scFv yields cells that have a similar phenotype to previously described CD19-specific CAR T cells.

### Example 3

### Antigen-Mediated CAR T Cell Proliferation, Cell Killing, and Cytokine Secretion

### 1. Methods

The purpose of this study was to evaluate CD20 CAR T cell performance in vitro for T cell proliferation, target cell killing, and effector cytokine production.

In this study, an apheresis sample was drawn from a healthy, informed, and compensated donor, and the T cells were enriched using the CD3 positive selection kit II in accord with the manufacturer's instructions (Stem Cell Technologies). T cells were activated using ImmunoCult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 ug of RNA encoding the TRC 1-2L.1592 meganuclease, which recognizes and cleaves the TRC 1-2 recognition sequence in the T cell receptor alpha constant locus, and were transduced with AAV packaged with construct 7260 or 7261 at an MOI of 25000 viral genomes/cell. Following transduction, cells were cultured in X-VIVO 15+5% FBS and 30 ng/ml IL-2 for a period of 5 days, at which point, the non-edited CD3+ cells were magnetically depleted using the CD3 positive selection kit (StemCell Technologies).

Flow cytometry was used to measure CD3 (clone UCHT1, BD Biosciences) and CAR expression to determine the frequency of TRAC knock-out and CAR knock-in cells. To detect CAR expression, two anti-idiotype clones (VM57 anti-muCD20 and VM4 anti-huCD20) were produced and conjugated to AlexaFluor647 in-house.

CAR T cells were placed into co-culture with target cells expressing CD20 or not expressing CD20. Both target cells were K562 lines. The CD20 negative line was simply parental K562 cells while the CD20+ line was K562 cells transfected with a CD20 expression vector (produced in-house), drug-selected for positive transfected cells, and FACS-sorted for the top 5% of expressors by mean fluorescence intensity on a Becton-Dickinson FACS Melody. This line was designated “K20.” The 7260 or 7261 CAR T cells were placed into culture with either K562 cells or K20 cells at target:effector ratios of 1:1, 3:1, or 9:1 in triplicate wells, where 1 is equal to 20,000 cells. The cultures were carried out for 6 days.

On day three, supernatant samples were obtained for analyses of cytokine secretion. On day 6, T cells and target cells were identified using anti-CD4 (clone OKT4, BioLegend), anti-CD8 (clone RPA-T8, BD Biosciences), and anti-CD20 (Clone 2H7), and enumerated using a Beckman-Coulter CytoFLEX-S flow cytometer. Supernatant samples were measured for IL-2, IFNγ, TNFα, and Granzyme B using the Ella Simple Plex cartridge reader (Protein Simple), and 4-plex array cartridges containing capture and detection reagents specific for the aforementioned cytokines.

### 2. Results

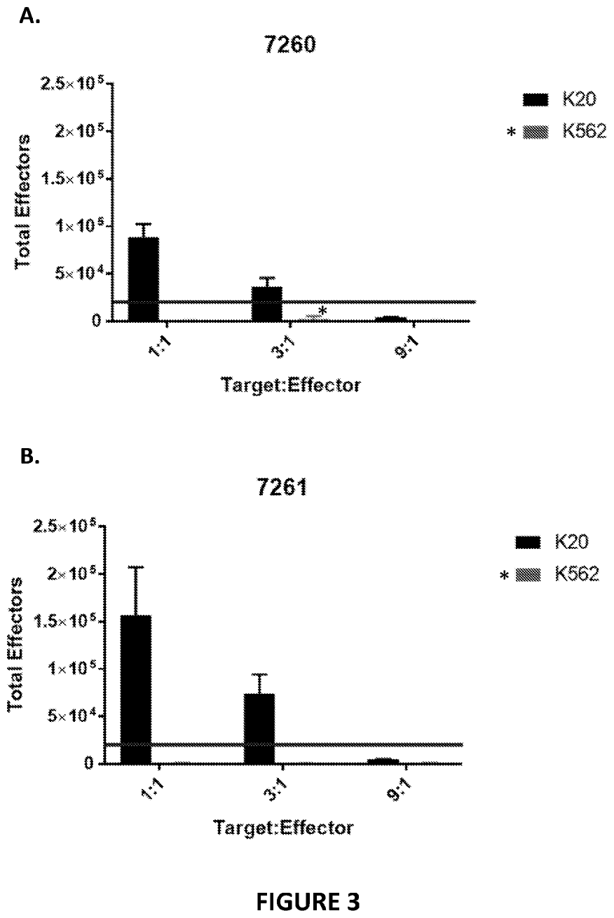

In this study, 20,000 CD20 CAR T cells were stimulated with either 20,000, 60,000, or 180,000 target cells and their expansion over the next six days was assessed and plotted (FIGS. 3A and 3B). Both 7260 and 7261 CAR T cells proliferated in response to CD20+ target cells, but not in response to unmanipulated K562 targets. Both CARs exhibited maximal expansion (4-7-fold) at T:E of 1:1, moderate expansion at 3:1, and no expansion at 9:1. At 1:1 ratios, 7261 CAR T cells displayed an approximately two-fold proliferative advantage over 7260 CAR T cells.

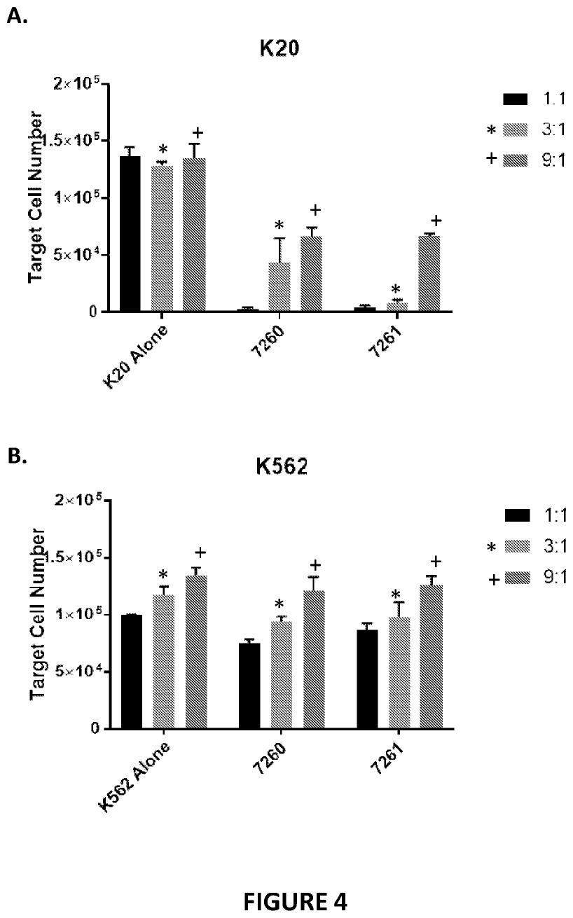

Both CAR T variants directed cytotoxic activity against CD20+ target cells but not K562 cells, as there were no differences in the number of surviving K562 when cultured in the presence or absence of CAR T cells (FIGS. 4A and 4B). By comparison, there were very few K20 cells that survived for 6 days in co-culture with either CAR T variant. Consistent with the proliferation data, 7261 CAR T cells displayed slightly more cytotoxic activity against K20 cells than 7260 CAR T cells, as appreciable numbers of targets were observed in 7260 co-cultures at 3:1 T:E ratios, but were not observed in 7261 co-cultures.

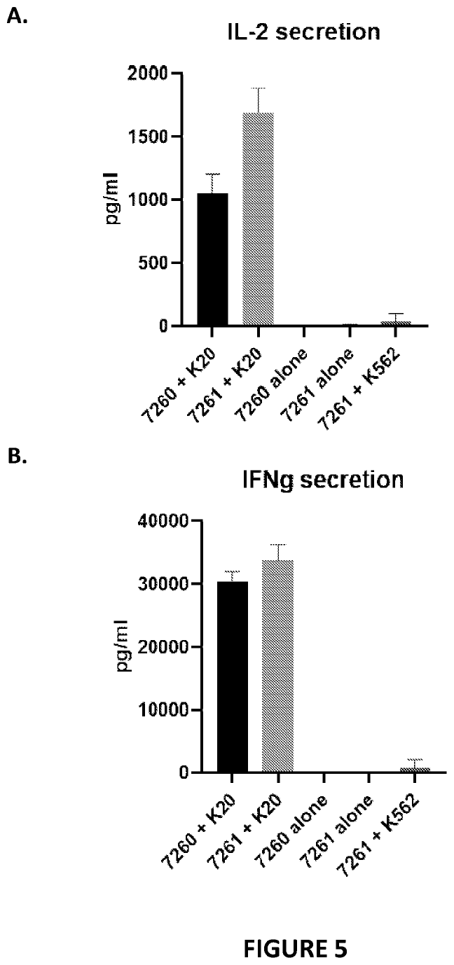

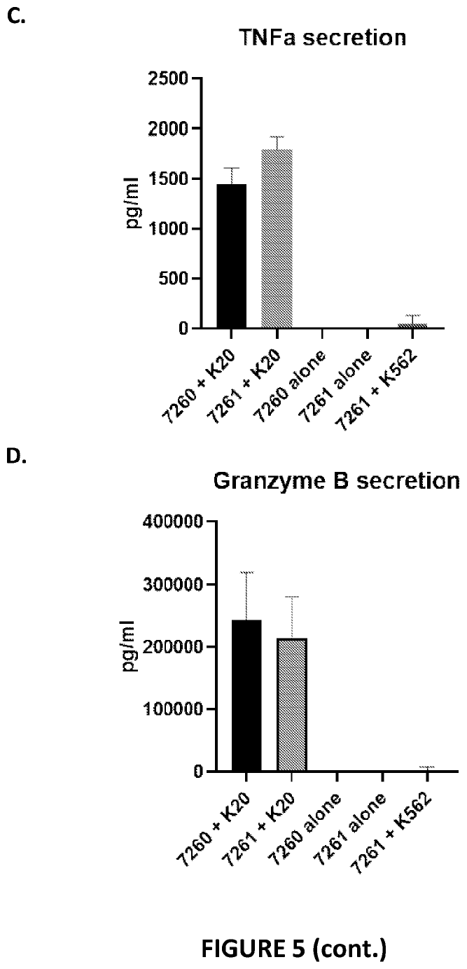

Both CAR T variants secreted effector cytokines in response to antigen encounter. High levels of IL-2, IFNγ, TNFα, and Granzyme B were detected in K20 co-cultures, but not K562 co-cultures or in cultures of CAR T cells alone (FIG. 5A-5D).

### 3. Conclusions

CAR T variants 7260 and 7261 exhibited proliferative, cytotoxic, and cytokine secretion responses following encounter with CD20+ target cells, but they did not do so in the absence of CD20. Slightly greater expansion and slightly more potent target killing responses were observed in 7261 CAR T cells.

### Example 4

### In Vivo Mouse Study with CD20-N6 CAR T Cells

### 1. Methods

In this study, an apheresis sample was drawn from a healthy, informed, and compensated donor, and the T cells were enriched using the CD3 positive selection kit II in accord with the manufacturer's instructions (Stem Cell Technologies). T cells were activated using ImmunoCult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 ug of RNA encoding the TRC 1-2L.1592 meganuclease, which recognizes and cleaves the TRC 1-2 recognition sequence in the T cell receptor alpha constant locus, and were transduced with AAV packaged with construct 7260 (encoding a CAR build from the muCD20 scFv) or 7261 (a CAR built from the huCD20 scFv) at an MOI of 25000 viral genomes/cell. Cultures were carried out for 5 days in complete X-VIVO-15 medium supplemented with 30 ng/ml IL-2 prior to conducting a flow cytometric analysis of CD3 (clone UCHT1, BD Biosciences) and CAR expression to determine the frequency of TRAC knock-out and CAR knock-in cells. To detect CAR expression, two anti-idiotype clones (VM57 anti-muCD20 and VM4 anti-huCD20) were produced and conjugated to AlexaFluor647 in-house. On day 5 post-transduction, the non-edited CD3+ cells were magnetically depleted using the CD3 positive selection kit (StemCell Technologies) and culture was carried out for an additional 3 days in X-VIVO 15 medium+5% FBS and 10 ng/ml of IL-15 and IL-21 (Gibco).

NOD.Cg-Prkdc^{scid} Il2rg^{tm1Wjl}/SzJ (NOD.scid.gamma chain KO, or NSG) mice were ordered from The Jackson Laboratory and were engrafted with Raji lymphoma cells expressing firefly luciferase. Each mouse was given 3×10⁶ Raji cells in 50% Matrigel (Corning) injected subcutaneously under the right flank. Tumor progression was monitored using twice-weekly caliper measurements and twice weekly luminescence imaging using the IVIS in vivo imaging system (Perkin Elmer). At day 14 following tumor implantation, doses of 1×10⁶ or 5×10⁶ CAR T cells made using either AAV6-7260 or AAV6-7261 were injected via the tail vein and tumor progression was monitored for an additional 80 days. Control groups which received vehicle or T cells not expressing CAR (TCR KO) were included in the study.

### 2. Results

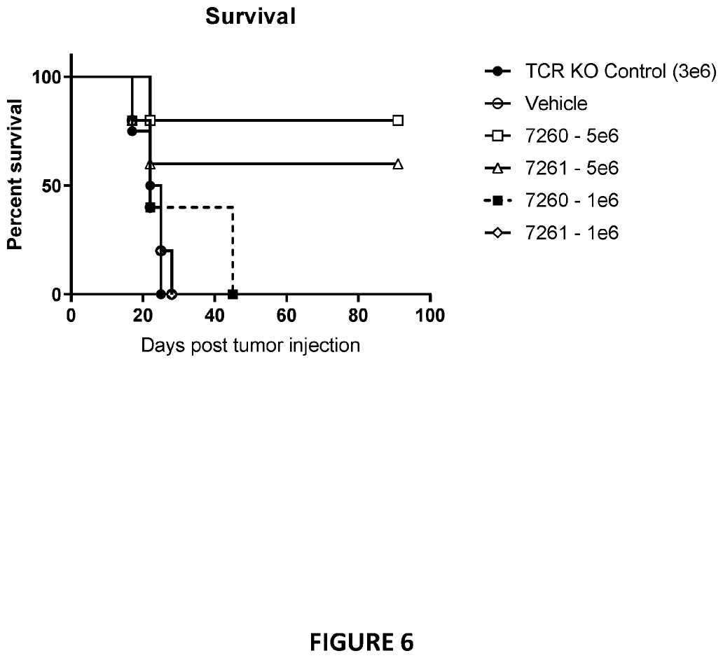

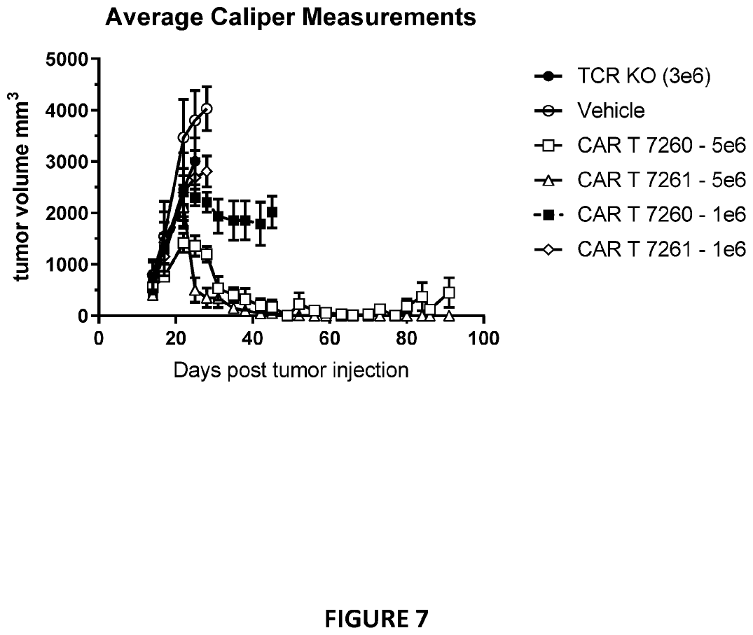

Treatment of tumor-bearing mice with either CAR variant resulted in lower luminescence signals, reduced tumor volumes, and increased survival. Although no significant survival advantage was observed at the low CAR T dose (median survival=22-24 days) compared to the control groups, the high doses of CAR T cells conferred significant protection. 3 mice receiving 7261 CAR T cells and 4 mice receiving 7260 CAR T cells survived for the entire 94 days. The three surviving mice in the 7261 group had no detectable tumor by either palpation or luminescence (FIG. 6). One of the four surviving mice in the 7260 group was likewise tumor-free. Caliper measurements confirmed these observations, as tumor sizes were reduced from above 1000 mm³ to non-measureable sizes by day 4. Tumor regression was only observed at the high doses of CAR T cells (FIG. 7).

### 3. Conclusions

Both variants of CAR T cells reduce the size of pre-established Raji lymphoma tumors and confer significant survival advantages at doses of 5×10⁶ cells/mouse. This suggests that either variant is active in in vivo lymphoma models.

### Example 5

### Comparison of huCD20-N6 to huCD20-N1

### 1. Methods

Three variants of the 7261 CAR sequence were constructed, each with different co-stimulatory signaling domains cloned into the intracellular domains to replace the N6 domain. Another novel signaling domain designed in-house N1 was cloned into construct 7364 (in place of N6) and native 4-1BB or an inactive 4-1BB mutant (DEL) were used to replace N6 in constructs 7362 and 7363, respectively (see Table 1 for construct design).

In this study, an apheresis sample was drawn from a healthy, informed, and compensated donor, and the T cells were enriched using the CD3 positive selection kit II in accord with the manufacturer's instructions (Stem Cell Technologies). T cells were activated using ImmunoCult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 ug of RNA encoding the TRC 1-2L.1592 meganuclease, which recognizes and cleaves the TRC 1-2 recognition sequence in the T cell receptor alpha constant locus, and were transduced with AAV packaged with construct 7261, 7362, 7363, or 7364 at an MOI of 25000 viral genomes/cell. Cultures were carried out for 5 days in complete X-VIVO-15 medium supplemented with 30 ng/ml IL-2 prior to conducting a flow cytometric analysis of CD3 (clone UCHT1, BD Biosciences) and CAR expression to determine the frequency of TRAC knock-out and CAR knock-in cells. To detect CAR expression, anti-idiotype clone VM4 anti-huCD20 was produced and conjugated to AlexaFluor647 in-house. In addition, the frequencies of CD4 and CD8 cells were determined using anti-CD4 clone OKT4, (BD Biosciences) and anti-CD8 clone HIT8a (BioLegend). A panel of surface markers were also measured to assess the degree to which the CAR T cells have differentiated in culture. Specifically, CD62L (clone SK11, BD), CD45RO (clone UCHL1 BioLegend), CD27 (Clone M-T271 BD), and CCR7 (clone G043H7, BioLegend) levels were measured. The following phenotypes were used to define the various populations:

CD62L^{HI}CD45RO^{Lo}=Central memory (CM)
CD62L^{HI}CD45RO^{HI}=Transitional memory (TM)
CD62L⁻CD45RO^{HI}=Effector memory (EM)

Positive CD27 and/or CCR7 expression was also used as an indicator of a central memory phenotype and to set the threshold of CD45RO Lo versus Hi expression (not shown).

### 2. Results

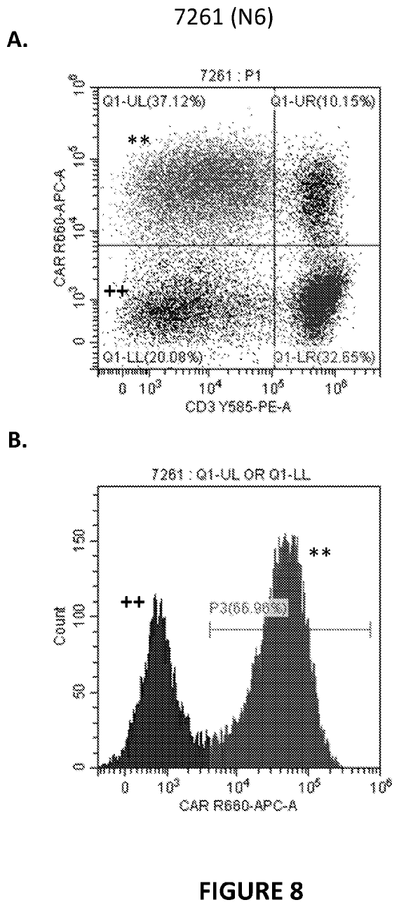

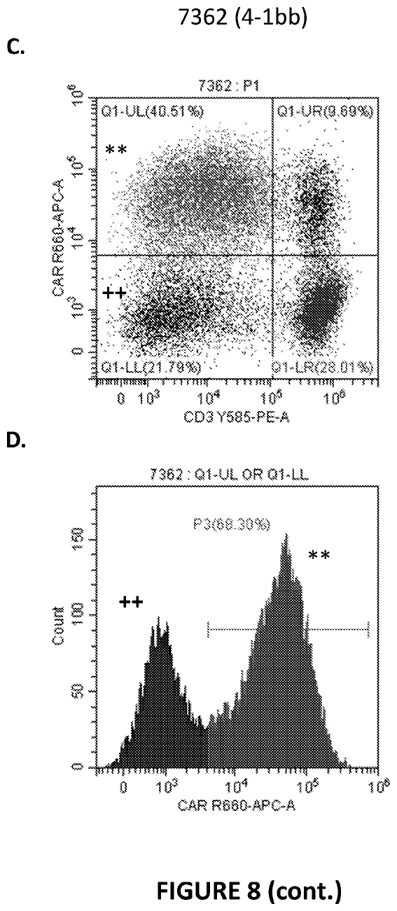

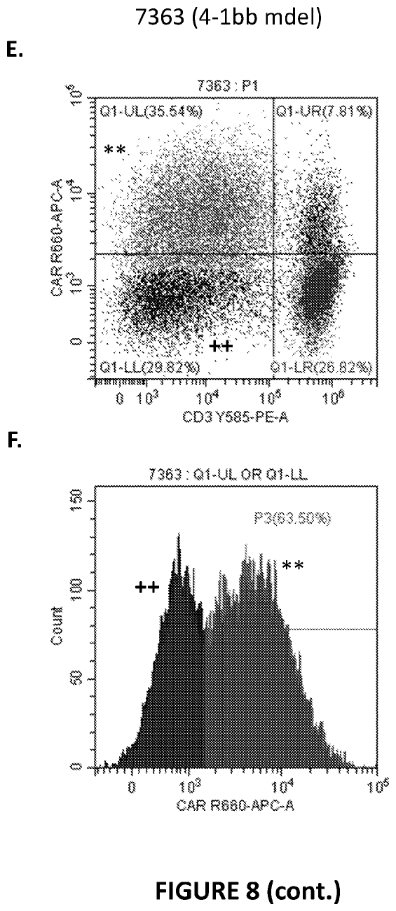

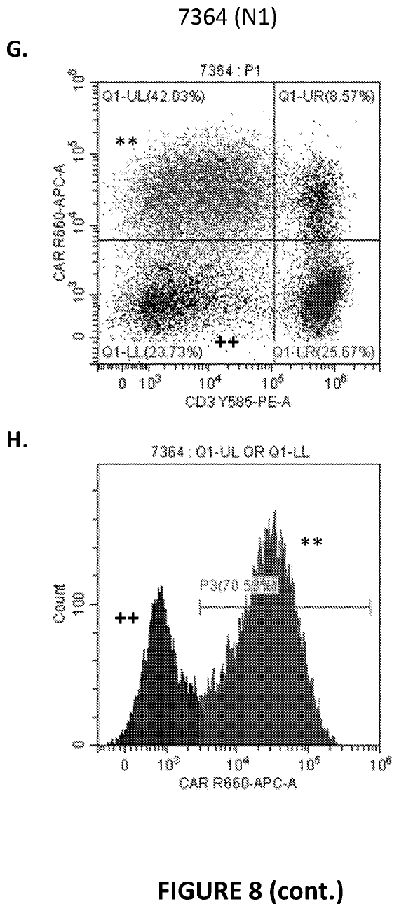

Flow cytometric evaluation of cell products revealed similar frequencies of TRAC-edited huCD20 CAR+ cells in cultures produced using the four different costimulatory signaling variants. As shown in FIG. 8, the frequency of CD3-CAR+ events ranged from 35.5-42% (FIGS. 8A, 8C, 8E, and 8G). When considering just the TRAC-edited cells (CD3−), the range of CAR+ cells was 63.5-70.5% (FIGS. 8B, 8D, 8F, and 8H). Table 2 provides CD4:CD8 ratios, and Table 3 provides the memory subset composition of the CD4 and CD8 CAR T cells. As was the case for knockout-knock-in rates, the frequency of CD4 and CD8 cells did not differ markedly from variant to variant, nor did the frequencies of the various memory subsets.

TABLE 2
CD4:CD8 ratio of CAR+ cells produced with
different costimulatory variants

| | CD4:CD8 | |
|---|---|---|
| Co-stimulatory Domain | CD4 | CD8 |
| 4-1BB mdel | 37 | 62 |
| N6 | 38 | 60 |
| N1 | 36 | 63 |
| 4-1bb | 39 | 59 |

TABLE 3
Memory Phenotype of CAR+ cells produced with
different costimulatory variants

| | Memory Phenotype | | | | |
|---|---|---|---|---|---|
| | CD62L/CD45RO | | | | |
| 4-1bb mdel | CM | TM | EM | CCR7 | CD27 |
| CD4 cells | 88 | 10 | 1 | 71 | 77 |
| CD8 cells | 98 | 1 | 0 | 52 | 81 |
| | CD62L/CD45RO | | | | |
| N6 | CM | TM | EM | CCR7 | CD27 |
| CD4 cells | 88 | 9 | 1 | 70 | 78 |
| CD8 cells | 98 | 1 | 0 | 50 | 82 |
| | CD62L/CD45RO | | | | |
| N1 | CM | TM | EM | CCR7 | CD27 |
| CD4 cells | 87 | 10 | 1 | 61 | 76 |
| CD8 cells | 98 | 1 | 0 | 48 | 80 |
| | CD62L/CD45RO | | | | |
| 4-1bb | CM | TM | EM | CCR7 | CD27 |
| CD4 cells | 83 | 14 | 1 | 72 | 57 |
| CD8 cells | 96 | 3 | 0 | 61 | 60 |

### 3. Conclusions

Data acquired from production runs of four costimulatory signaling domain variants indicate that there is no difference in the phenotype of the cells produced using either of the CAR vectors. Any potential differences observed in their function are not likely to be ascribable to phenotypic differences that are acquired during production.

### Example 6

### Stress Test Proliferation and Cell Killing

### 1. Methods

In this study, an apheresis sample was drawn from a healthy, informed, and compensated donor, and the T cells were enriched using the CD3 positive selection kit II in accord with the manufacturer's instructions (Stem Cell Technologies). T cells were activated using ImmunoCult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 ug of RNA encoding the TRC 1-2L.1592 meganuclease, which recognizes and cleaves the TRC 1-2 recognition sequence in the T cell receptor alpha constant locus, and were transduced with AAV packaged with construct 7261 (containing the N6 signaling domain), 7362 (4-1BB signaling domain), 7363 (4-1BB DEL signaling domain), or 7364 (N1 signaling domain), all at an MOI of 25000 viral genomes/cell. Following transduction, cells were cultured in X-VIVO 15+5% FBS and 30 ng/ml IL-2 for a period of 5 days, at which point, the non-edited CD3+ cells were magnetically depleted using the CD3 positive selection kit (StemCell Technologies). Flow cytometry was used to measure CD3 (clone UCHT1, BD Biosciences) and CAR expression to determine the frequency of TRAC knock-out and CAR knock-in cells. To detect CAR expression, an anti-idiotype antibody (VM4 anti-huCD20) was produced and conjugated to AlexaFluor647 in-house. CAR T cells were placed into co-culture with K562 cells transfected with a CD20 expression vector (produced in-house), drug-selected for positive transfected cells, and FACS-sorted for the top 5% of expressors by mean fluorescence intensity on a Becton-Dickinson FACS Melody. This like was designated “K20.”

CAR T cells were placed into culture in triplicate wells of a 96 well plate with K20 cells at a target:effector ratio of 1:1, where 1=20,000 cells. At days 7, 11, 14, 17, and 21, the cultures were sampled and T cells and target cells were identified using anti-CD4 (clone OKT4, BioLegend), anti-CD8 (clone RPA-T8, BD Biosciences), and anti-CD20 (Clone 2H7), and enumerated using a Beckman-Coulter CytoFLEX-S flow cytometer. Immediately thereafter, fresh K20 target cells were added to the cultures such that a 1:1 ratio was re-established at each time point.

### 2. Results

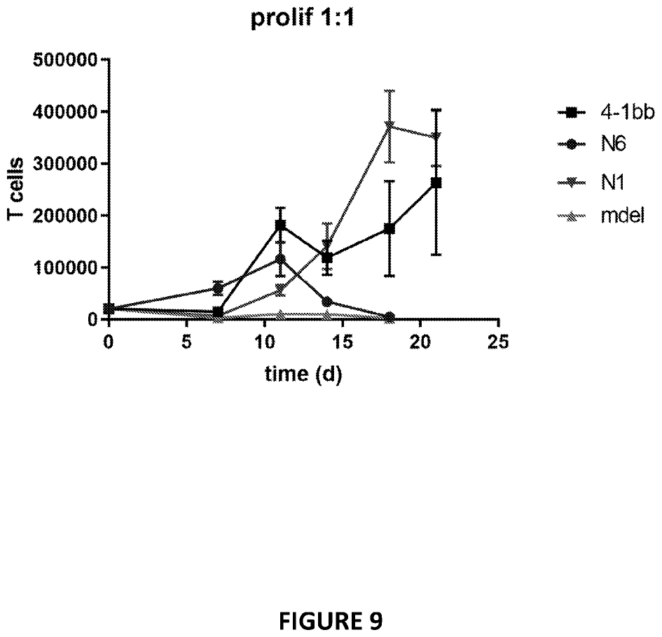

Increases to the number of T cells were observed in the first 11 days of culture for all CAR T variants except for the construct expressing the 4-1BB mdel signaling domain. As 4-1BB mdel is a functionally inactivated signaling domain, this was expected. At d11 of culture, the each signal peptide demonstrated increases in T cell numbers. Between d11 and the end of the experiment, the number of T cells in the N6 culture steadily decreased while the T cells in the 4-1BB and N1 cultures increased on average (FIG. 9).

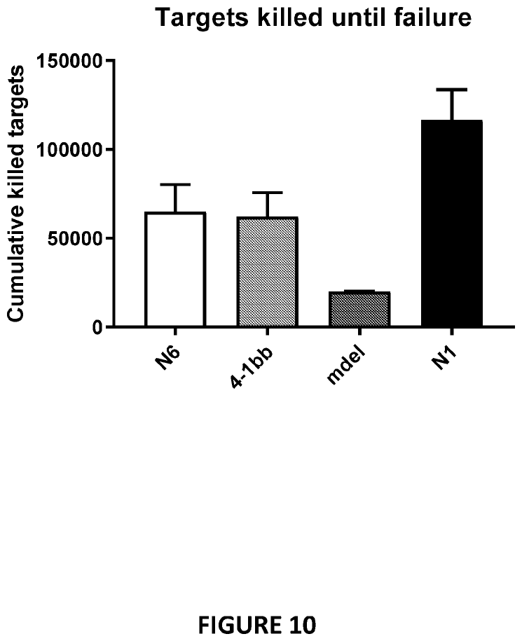

Because the experiment featured the addition of a specified number of fresh target cells at each time point, and the number of surviving target cells was calculated at each analysis, the total number of targets killed by the CAR T signaling variants could be plotted. As displayed in FIG. 10, the 4-1BB, N1, and N6 co-stimulatory domains were functional in killing target cells.

### 3. Conclusions

In this examination of CAR T responses to repeated antigen encounters, CAR T cells produced with the 4-1BB, N1, and N6 co-stimulatory signaling domains exhibited sustained proliferation and targeted cell killing.

### Example 7

### Specificity and Activity of CD20 CAR T Cells Prepared from Multiple Donors

### 1. Background

This study sought to analyze the specificity and activity of CD20-specific CAR T cells of the invention, prepared from three separate healthy human donors, against CD20+ and CD20− target cell lines in vitro. Furthermore, the phenotype of CD20 CAR T cells was evaluated. The CD20-specific CARs utilized in these CAR T cells include an scFv (oriented VL-linker-VH) comprising the VH region (SEQ ID NO: 5) and VL region (SEQ ID NO: 7) of the huCD20 antibody connected by a polypeptide linker (SEQ ID NO: 25). The full scFv comprised an amino acid sequence set forth in SEQ ID NO: 47. The CAR further included a CD8 alpha hinge domain (SEQ ID NO: 27), a CD8 alpha transmembrane domain (SEQ ID NO: 29), an N6 co-stimulatory domain (SEQ ID NO: 23), and a CD3 zeta signaling domain (SEQ ID NO: 31). The CAR further included an N-terminal CD8 signal peptide set forth in SEQ ID NO: 33. The full CD20 CAR comprised an amino acid sequence set forth in SEQ ID NO: 75, and was encoded by a nucleic acid sequence set forth in SEQ ID NO: 76. The CD20 CAR T cells used for this study were generated as full-scale demonstration runs which used the same process, scale, and comparable materials to be used for current Good Manufacturing Practices manufacturing.

### 2. Methods

### Production of CD20 CAR T Cells

CD20 CAR T cells were prepared as previously described in Example 2 from T cells obtained from three different healthy human donors (CD20Donor1, CD20Donor2, and CD20Donor3, respectively). Cryopreserved CD20 CAR T cells were thawed and added to X-VIVO 15 medium supplemented with 5% fetal bovine serum (FBS). The cell suspension was centrifuged and the supernatant decanted. The cells were resuspended in X-VIVO 15 medium supplemented with 5% FBS and 10 ng/mL each of IL-15 and IL-21 and plated in a sterile tissue culture flask and placed in an incubator overnight.

Target K20 (CD20+) and K562 (CD20-) cells were thawed, washed, and resuspended in X-VIVO 15 medium and incubated overnight.

### Immunophenotyping

Immunophenotyping of the three CD20 CAR T cell batches was conducted. In brief, an aliquot of cells from each batch was washed in phosphate buffered saline (PBS), centrifuged, and then stained with an antibody cocktail in PBS for 15 minutes at room temperature. Samples were then washed twice in PBS, resuspended in fresh PBS, and analyzed on a flow cytometer to collect data for frequency of CD3− cells, frequency of CAR+ cells, CD4:CD8 ratio, and frequency of T cell memory subpopulations.

### CD20 CAR T Cell Co-Culture

Target K20 cells (human K562 cells engineered to express CD20) were used to stimulate CD20 CAR T cell responses and unmanipulated human K562 cells which do not express CD20 were included as negative controls. CD20 CAR T cells were cocultured with K20 cells in X-VIVO 15 medium supplemented with 5% FBS at E:T ratios (CD20 CAR T cells:K20 cells) of 1:1 (2×10⁴:2×10⁴), 1:3 (2×10⁴:6×10⁴), and 1:9 (2×10⁴:18×10⁴). CD20 CAR T cells were cocultured with K562 cells at an E:T of 1:1. Cocultured CD20 CAR T cells and target cells were incubated for 48 hours and assessed for cytokine release and incubated for an additional 3 days and then assessed for proliferation and cytotoxicity.

### Assessment of Cytokine Release

After 48 hours of coculture, 50 μL of supernatant was removed from each coculture well and stored at −20° C. until analysis. Coculture supernatants were thawed and diluted at a 1:10 ratio in manufacturer's diluent and a 4-plex cartridge was loaded according to manufacturer's instructions (50 μL/well for sample wells, 1 mL/well for wash buffer). Levels of IFNγ, IL-2,

IL-6, and TNFα were measured in supernatants on a ProteinSimple Ella plate reader. Activity of CD20 CAR T cells against K20 target cells was measured in triplicate coculture wells. Activity of CD20 CAR T cells against K562 control cells was measured in duplicate coculture wells.

### In Vitro Assessment of Proliferation and Cytotoxicity

Proliferation and cytotoxicity samples were prepared at Day 5. Cocultures were resuspended by pipetting and 140 μL samples were removed and prepared for flow cytometric analyses. Samples were incubated for 15 minutes at 4° C. with 100 μL of PBS containing:
1 μL anti-CD8 BV421
0.5 μL anti-CD4 FITC
0.5 μL anti-CD20 PE
0.25 μL Ghost Dye 510
   After incubation, 200 μL of PBS was added to the samples and the cells were pelleted by centrifugation. Cells were resuspended in 120 μL of PBS. Sample data were acquired immediately after PBS resuspension on a Beckman-Coulter CytoFLEX-S flow cytometer. Cell count data were captured and exported for analysis.

### 3. Results

### Phenotype of CD20 CAR T Cells

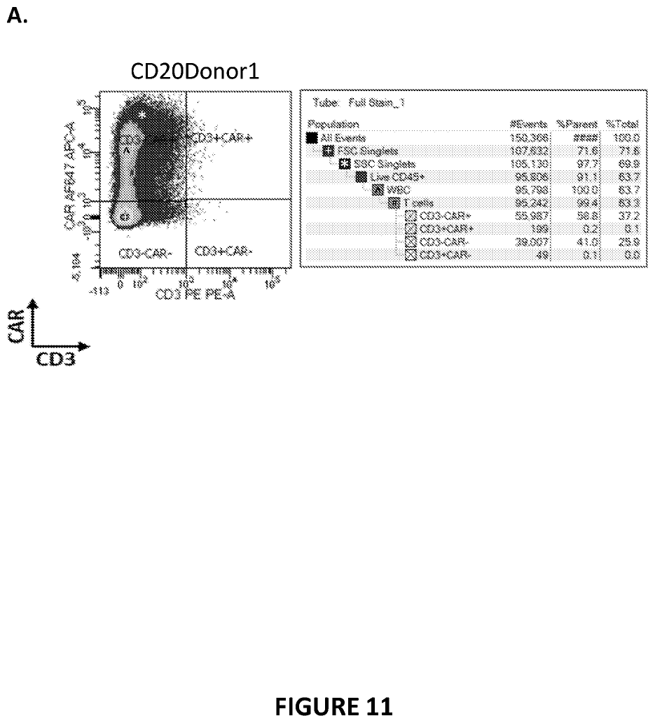

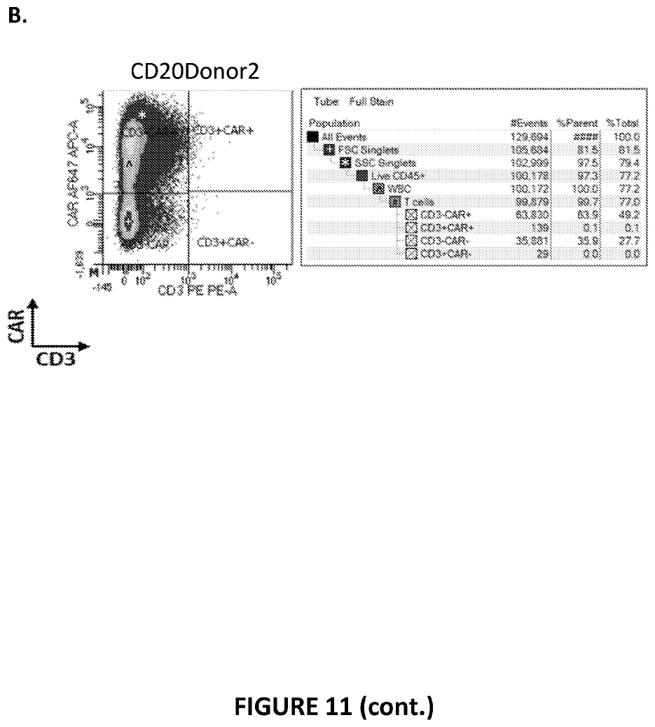

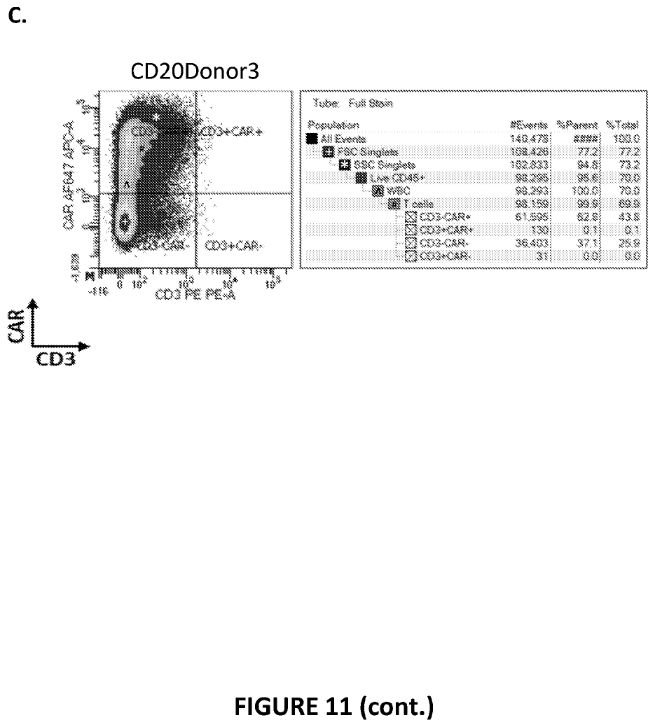

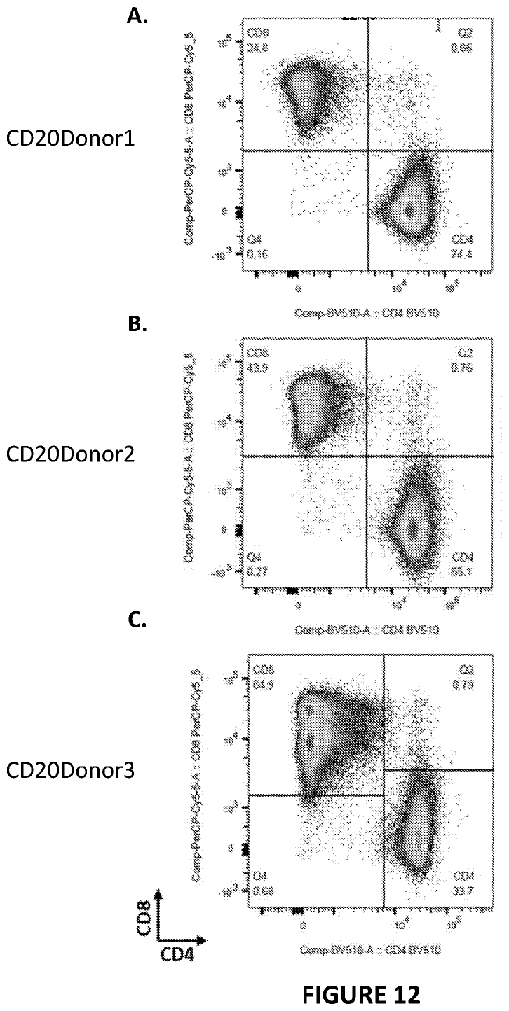

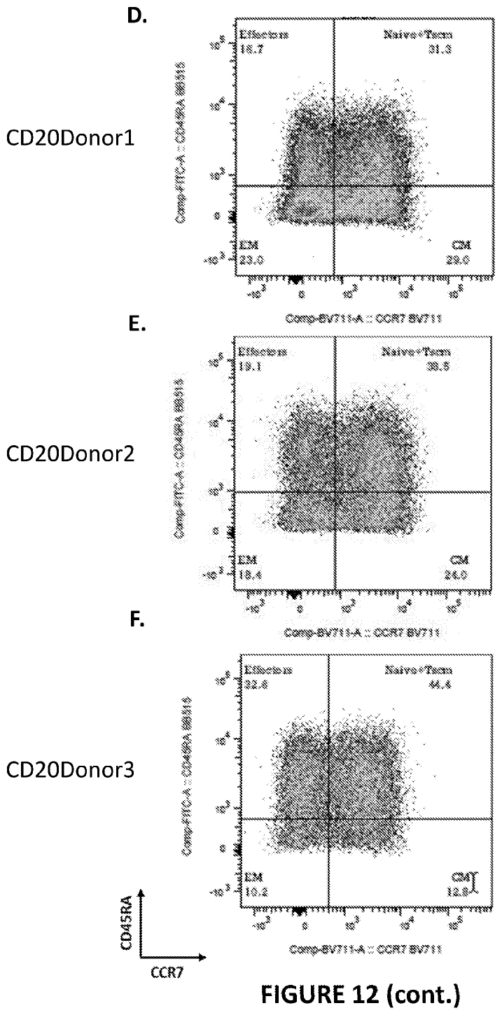

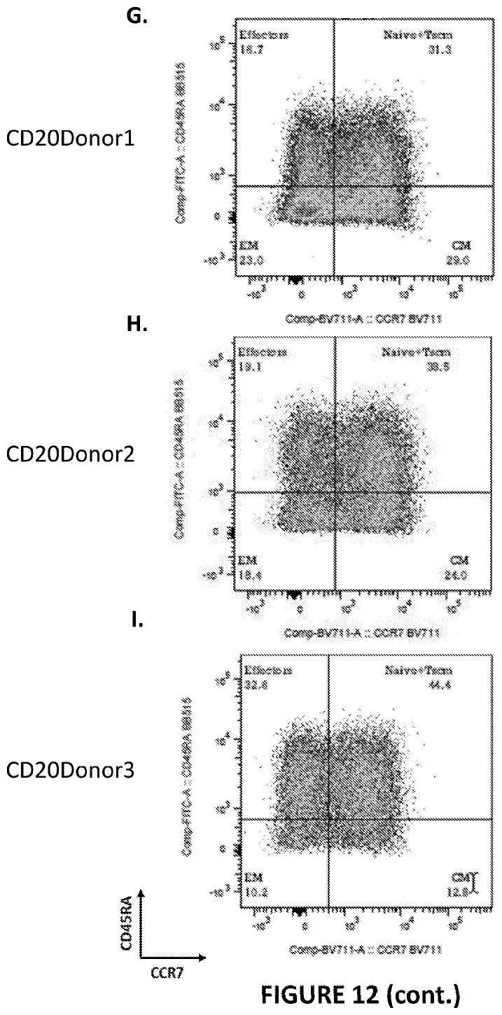

Three batches of CD20 CAR T cells (CD20Donor1, CD20Donor2, and CD20Donor3) were analyzed by flow cytometry to determine the percentage of T cells that are CD3−, CAR+, CD4+, CD8+, naïve (Tn), central memory (Tcm), and effector memory (Tem) phenotypes. Flow cytometry results for all 3 batches of CD20 CAR T cells are summarized in Table 4 below and flow cytometry plots are presented in FIG. 11 and FIG. 12.

Flow cytometry results demonstrate that >99% of the cells are CD3-, of which>50% are CD3-CAR+(range: 58.8% to 63.9%). The CD4:CD8 ratios of CD3-CAR+ cells ranged from 0.52 to 3. The majority of CD4+CAR+ cells are represented by a combination of Tn and Tcm phenotypes. This data profile shows that the process consistently generates an enriched population of CD3-CAR+ T cells with a desirable composition and phenotype.

TABLE 4
Overview of CD20 CAR T cell characterization

| Parameter | CD20Donor1 | CD20Donor2 | CD20Donor3 |
|---|---|---|---|
| CD3−(%) | 99.8 | 99.8 | 99.9 |
| CD3−CAR⁺ (%) | 58.8 | 63.9 | 62.8 |
| KI (% of KO) | 58.9 | 64.0 | 62.9 |
| CD4:CD8 ratio | 3.00 | 1.26 | 0.519 |
| (CD3−CAR⁺) | | | |
| CD4⁺CD3− | 74.4 | 55.1 | 33.7 |
| CAR⁺(%) | | | |
| CD8⁺CD3− | 24.8 | 43.9 | 64.9 |
| CAR⁺(%) | | | |
| CD4⁺CCR7⁺ | 60.3 | 62.5 | 57.2 |
| (%) | | | |
| CD8⁺CCR7⁺ | 45.0 | 44.4 | 33.7 |
| (%) | | | |
| Viability(%) | 92.2 | 92.6 | 77.8 |

### CD20 CAR T Cell Activity—Proliferation

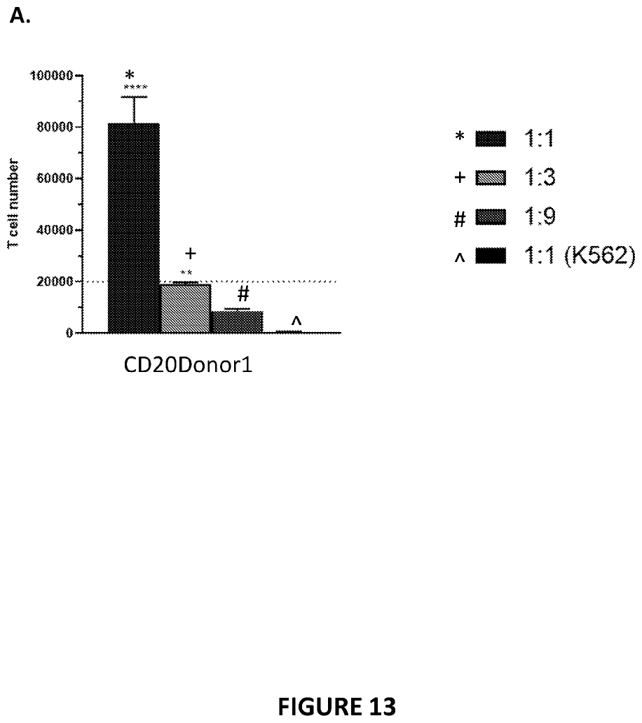

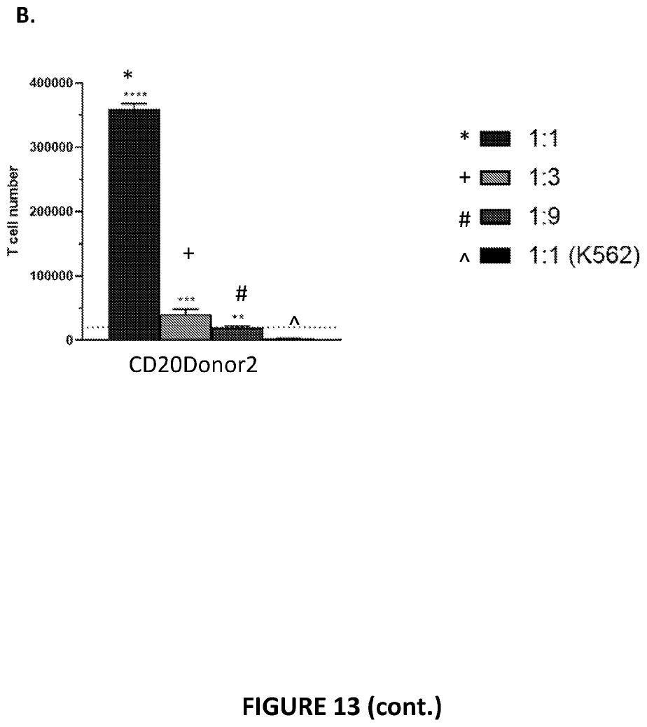

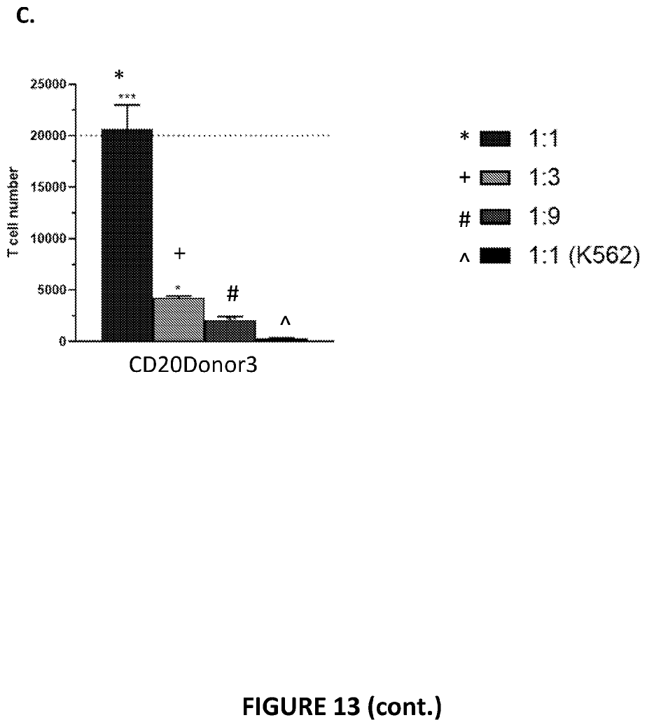

After 5 days of coculture, CD20 CAR T cells from 3 different donors proliferated in response to stimulation by CD20+ K20 target cells at an E:T ratio of 1:1 as shown in FIG. 13. Batch CD20Donor2 (FIG. 13B) demonstrated the highest levels of expansion when compared to batches CD20Donor1 (FIG. 13A) and CD20Donor3 (FIG. 13C), which showed no proliferation at E:T ratios of 1:3 and 1:9. As expected, CD20 CAR T cells did not proliferate in response to coculture with CD20− K562 cells.

### CD20 CAR T Cell Activity—Cytotoxicity

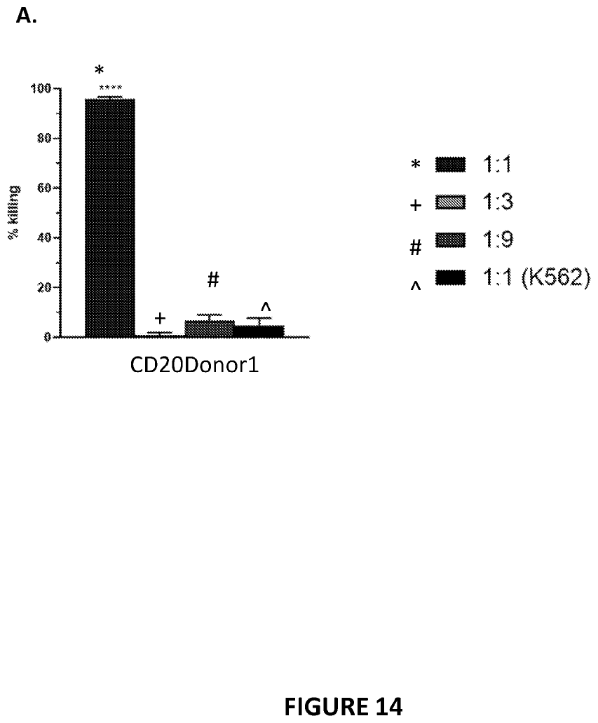

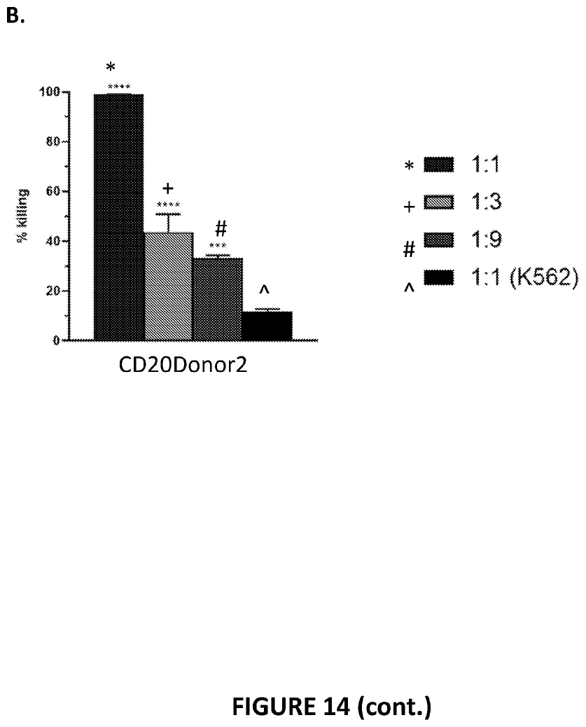

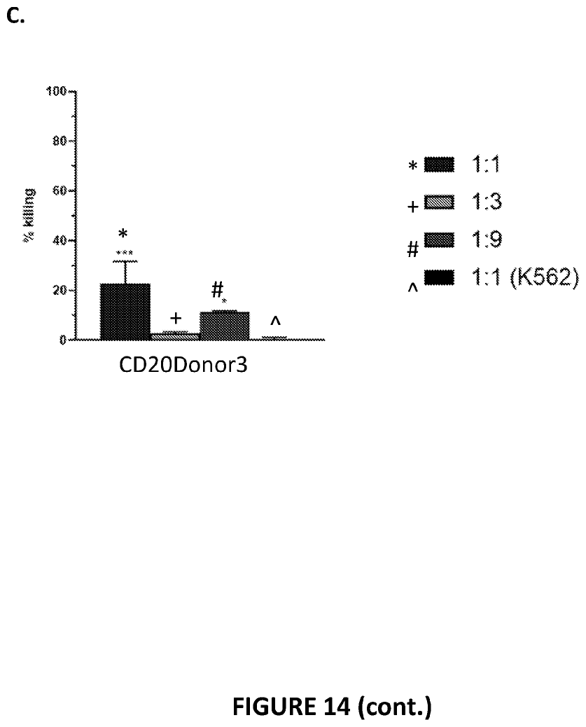

The cytotoxic potential of CD20 CAR T cells from 3 different donor batches was evaluated after 5 days of coculture in the presence of CD20+ K20 target cells and CD20− K562 cells. FIG. 14 shows CD20 CAR T cell-mediated cytotoxic killing of CD20+ K20 cells in vitro at E:T ratios ranging from 1:1 to 1:9. CD20donor1 CART cells (FIG. 14A) demonstrated the highest levels of cytotoxicity in response to CD20+ K20 target cells at all E:T ratios when compared to batches CD20donor2 CAR T cells (FIG. 14B) and CD20donor3 CAR T cells (FIG. 14C). Target cell killing was not observed when CD20 CAR T cells from any batch were cocultured with CD20− K562 target cells.

### CD20 CAR T Cell Activity—Cytokine Response

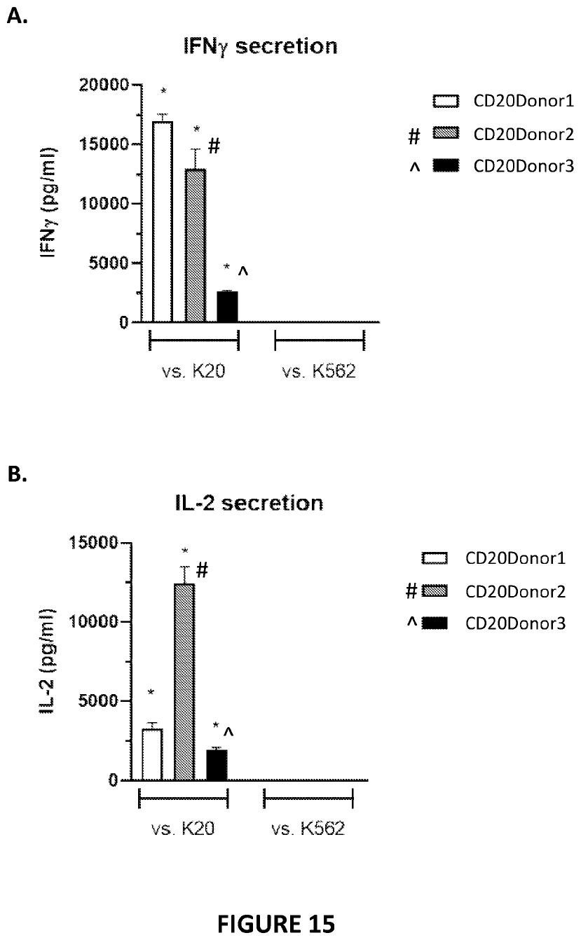

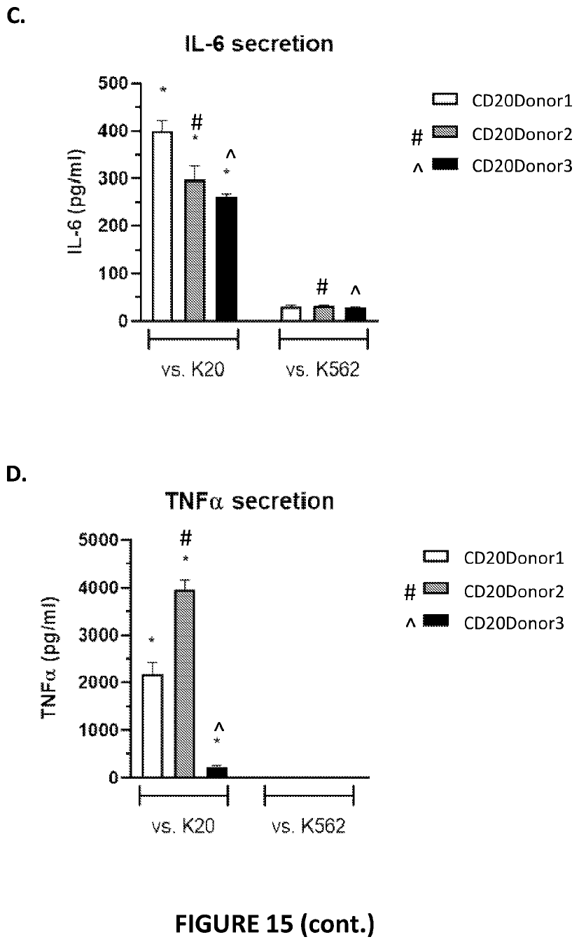

The cytokine response of CD20 CAR T cells from 3 different donor batches was evaluated after 2 days of coculture in the presence of CD20+ K20 target cells and CD20− K562 cells by testing cell culture supernatants by multiplex enzyme-linked immunosorbent assays. CD20 CAR T cells produce the cytokines IFNγ, IL-2, IL-6, and TNFα when cocultured with CD20+ K20 target cells (FIG. 15). In contrast, CD20 CAR T cells cocultured with CD20− K562 target cells exhibited minimal production of cytokines.

### 4. Conclusions

The data provided in these studies demonstrates that the CD20 CAR T cells generated from 3 independent donors were greatly enriched for CD3− cells with >50% CD3− CAR+ T cells and had a desirable composition and phenotype (CD4:CD8 ratio≥0.5:1, Tn+Tcm≥50%). These CAR T cell products all demonstrated activity specifically when cocultured with CD20+ cells and not in the presence of CD20− control cells. Overall, these results confirm the specificity and activity of the CD20 CAR T cells towards CD20+ target cells.

### Example 8

### In Vivo Efficacy of CD20 CAR T Cells in Subcutaneous Mantle Cell Lymphoma Model

### 1. Background

This in vivo study evaluated CD20 CAR T cells from Donor 2, as described above in Example 7 (i.e., CD20Donor2), for antitumor efficacy in a murine xenograft subcutaneous model of mantle cell lymphoma (MCL) for 45 days. The antitumor efficacy of the 3 CD20 CAR T cell batches was assessed at doses ranging from 1×10⁶ to 1×10⁷ cells per animal. Efficacy was determined by inhibition of tumor growth assessed by caliper measurements, body weight, and survival in comparison to control.

### 2. Methods

In this in vivo efficacy study, 1×10⁶ Granta-519 tumor cells were implanted subcutaneously on the right flank of female NSG mice. Once tumors reached an average size of 80 to 120 mm³, dosing began (Day 1, 16 days postimplantation). Mice were dosed IV with either vehicle control, CD3− control T cells, or CD20 CAR T cells (Table 5).

TABLE 5
In vivo efficacy study design (Granta-519-PRCB-e200)

| | | Tumor | | Dose | | |
|---|---|---|---|---|---|---|
| Group | n | implantation | Treatment (IV) | (cell/animal) | Body Weight | Caliper |
| 1 | 6 | 1 × 10⁶ cells | Vehicle² | | QD × 5, then | Biweekly |
| | | | | | biweekly to end | |
| 2 | 6 | 1 × 10⁶ cells | CD3-T cells | 5 × 10⁶ | QD × 5, then | Biweekly |
| | | | (NP11)³ | | biweekly to end | |
| 3 | 6 | 1 x 10⁶ cells | CD20 CAR T | 1 × 10⁶ | QD × 5, then | Biweekly |
| | | | low (NP10)⁴ | | biweekly to end | |
| 4 | 6 | 1 x 10⁶ cells | CD20 CAR T | 5 × 10⁶ | QD × 5, then | Biweekly |
| | | | mid (NP10)⁴ | | biweekly to end | |
| 5 | 6 | 1 x 10⁶ cells | CD20 CAR T | 1 x 10⁷ | QD × 5, then | Biweekly |
| | | | high (NP10) ⁴ | | biweekly to end | |

### Mice

Female NSG mice (NOD.Cg-Prkdc^{scid}Il2^{rgtmIWjl}/SzJ, The Jackson Laboratory) were 10-weeks old with body weights ranging from 19.2 to 25.9 grams at the beginning of the study (Day 1 of dosing).

### Granta-519 Cells

Human Granta-519 cells (ACC 342, DSMZ) were established from the peripheral blood taken in 1991 at relapse of a high-grade B-NHL (leukemic transformation of MCL, stage IV) diagnosed in a 58-year-old Caucasian woman with previous history of cervical carcinoma. Frozen cells were thawed and cultured according to supplier's recommendation in Dulbecco's Modified Eagle Medium (high glucose) containing 10% fetal bovine serum, 2 mM glutamine, 100 units/mL penicillin G sodium, 100m/mL streptomycin sulfate, and 25 μg/mL gentamicin, and incubated in 5% CO₂ at 37° C.

### Test and Control Products

CD20 CAR T cells (CD3-CAR+) and TCR knock-out control T cells (CD3-) were produced as described in Example 2. The cells were supplied frozen and formulated in cryopreservation media (48.0% normal saline, 2.0% human serum albumin (HSA), 47.5% Cryostor CS10, 2.5% dimethyl sulfoxide (DMSO), with the final DMSO concentration at 7.5%). Drug product diluent (Plasmalyte and 2.0% HSA) served as the vehicle. Pre- and post-injection viability was 90.9% and 88.4%, respectively, for CD3− control T cells. Pre-injection viability for CD20 CAR T cell doses ranged from 84.0% to 87.9% and post-injection viability ranged from 82.9 to 87.4%.

### Subcutaneous Tumor Cell Injection and Tumor Growth

Granta-519 cells were harvested during log phase growth and resuspended in RPMI medium at a concentration of 1×10⁷ cells/mL. Each mouse was injected subcutaneously into the right flank with 1×10⁶ Granta-519 cells (in a 0.1 mL suspension) into the right flank of each animal. Tumors were monitored as their volumes approached the target range of 80 to 120 mm³. Tumors were measured twice a week for the duration of the study in 2 dimensions using calipers, and volume was calculated using the formula:

Tumor Volume (mm³)=(length×width²)/2

Sixteen days after tumor cell implantation (designated as Day 1 of the study), animals were sorted into 5 groups (n=6 per group) with individual tumor volumes of 75 to 144 mm³, and group mean tumor volumes from 97 to 100 mm³.

### Treatment

Animals were randomized to 6 animals per group based on tumor volume. Animals were administered 5×10⁶ CD3− control T cells, or 1×10⁶, 5×10⁶, and 1×10⁷ CD20 CAR T cells after tumors reached an average size of 80 to 120 mm³. CD20 CAR T cell dosing began on Day 1 of the study, which was 16 days postimplantation of Granta-519 cells. Dosing was initiated according to the treatment plan summarized in Table 5 above.

### Endpoint and Tumor Growth Delay Analysis

Individual animals were euthanized when tumor volume reached 2000 mm³ or on the last day of the study (Day 62), whichever came first. Animals that exited the study for tumor volume endpoint were documented as euthanized for tumor progression, with the date of euthanasia. The time to endpoint (TTE) for analysis was calculated for each mouse by the following equation:

TTE=(log₁₀(endpoint volume)−b)/m

where TTE is expressed in days, endpoint volume is expressed in mm³, b is the intercept, and m is the slope of the line obtained by linear regression of a log-transformed tumor growth data set.

The data set consisted of the first observation that exceeded the endpoint volume used in analysis and the 3 consecutive observations that immediately preceded the attainment of this endpoint volume. The calculated TTE is usually less than the tumor progression date, the day on which the animal was euthanized for tumor size. Animals with tumors that did not reach the endpoint volume were assigned a TTE value equal to the last day of the study (Day 62). In instances in which the log-transformed calculated TTE preceded the day prior to reaching endpoint or exceeded the day of reaching tumor volume endpoint, a linear interpolation was performed to approximate the TTE.

Any animal classified as having died from non-treatment related (NTR) causes due to accident or due to unknown etiology were excluded from TTE calculations (and all further analyses).

Animals classified as TR (treatment-related) deaths or NTR deaths due to metastasis were assigned a TTE value equal to the day of death.

Treatment outcome was evaluated from tumor growth delay (TGD), which is defined as the increase in the median TTE in a treatment group compared to the control group:

TGD=T−C,

expressed in days, or as a percentage of the median TTE of the control group:

% TGD=(T−C)/C×100

Where T=median TTE for a treatment group and C=median TTE for the designated control group.

### Median Tumor Volume and Criteria for Regression Responses

Treatment efficacy was also determined from the tumor volumes of animals remaining in the study on the last day (Day 62) and from the number and magnitude of regression responses. The MTV (n) is defined as the median tumor volume on Day 62 in the number of evaluable animals remaining, n, whose tumors have not attained the volume endpoint. Treatment may cause partial regression (PR) or complete regression (CR) of the tumor in an animal.

In a PR response, the tumor volume is 50% or less of its Day 1 volume for 3 consecutive measurements during the course of the study, and equal to or greater than 13.5 mm³ for 1 or more of these 3 measurements. In a CR response, the tumor volume is less than 13.5 mm³ for 3 consecutive measurements during the study. Animals were scored only once during the study for a PR or CR event and only as a CR if both PR and CR criteria were satisfied. Any animal with a CR response at the end of the study was additionally classified as a tumor-free survivor.

### Clinical Observations

Animals were weighed daily from Day 1 to Day 5, then twice a week until the completion of the study. The mice were observed frequently for overt signs of any TR side effects, and clinical observations were recorded. Individual body weight was monitored as per protocol, and any animal with weight loss exceeding 30% for 1 measurement or exceeding 25% for 3 consecutive measurements was euthanized as a TR death (for treated groups).

Group mean body weight loss was also monitored. An animal death was classified as TR if the death was attributable to treatment side effects as evidenced by clinical signs and/or necropsy. A TR classification was also assigned to deaths by unknown causes during the dosing period or within 14 days of the last dose. A death was classified as NTR if there was no evidence that death was related to treatment side effects.

### Statistical and Graphical Analyses

Prism (GraphPad) for Windows 8.1.1 was used for graphical presentations and statistical analyses. Study groups experiencing toxicity beyond acceptable limits (>20% group mean body weight loss or greater than 10% TR deaths) or having fewer than 5 evaluable observations, were nonevaluable and not included in statistical analyses. Prism summarizes test results as not significant at p>0.05, significant (symbolized by “*”) at 0.01<p≤0.05, very significant (“**”) at 0.001<p<0.01, and extremely significant (“***”) at p<0.001. Because tests of statistical significance do not provide an estimate of the magnitude of the difference between groups, all levels of significance were described as either significant or not significant within the text of this report.

The log rank test, which evaluates overall survival experience, was used to analyze the significance of the differences between the TTE values of 2 groups. Logrank analysis includes the data for all animals in a group except those assessed as NTR deaths. Two-tailed statistical analyses were conducted at significance level p=0.05 and were not corrected for multiple comparisons.

Scatter plots were constructed to show TTE values for individual mice, by group. Group median and mean tumor volumes were plotted as a function of time. When an animal exited the study due to tumor size, the final tumor volume recorded for the animal was included with the data used to calculate the mean volume at subsequent time points.

Kaplan-Meier plots, which uses the same TTE data set as the log rank test, shows the percentage of animals in each group remaining in the study versus time.

Group body weight changes over the course of the study were plotted as percent mean change from Day 1. Tumor growth and body weight plots excluded the data for animals assessed as NTR deaths and were truncated when fewer than 50% of the animals in a group remained in the study.

### 3. Results

### Antitumor Efficacy of CD20 CAR T Cells

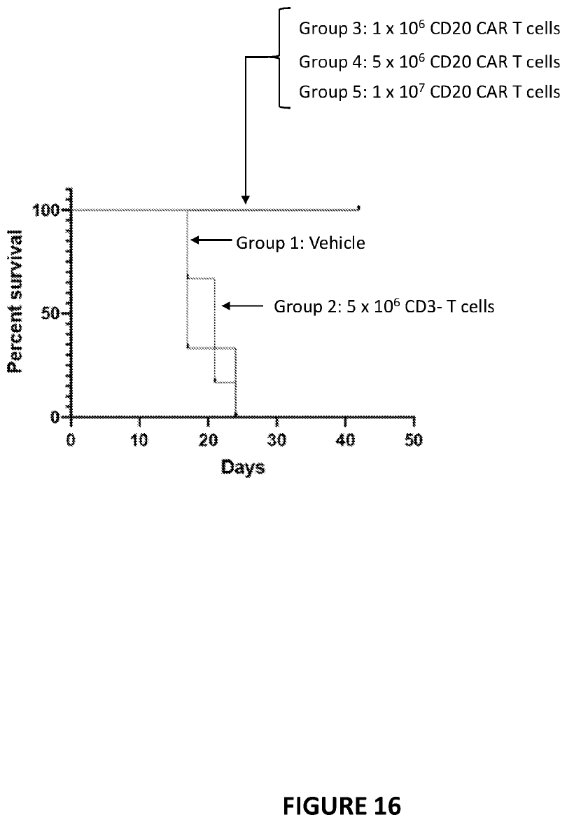

All groups were monitored for survival and tumor volume over time as described above. CD20 CAR T cells conferred significant survival advantages at all doses over vehicle control or CD3− control T cell administration (FIG. 16). On Day 17, four animals each in the vehicle control and CD3− control T cell groups died due to tumor metastasis. Additional deaths due to tumor metastasis occurred on Day 21 (1 animal, CD3− control T cell group), Day 23 (1 animal, vehicle control group), and Day 24 (1 animal, CD3− control T cell group). One animal from the vehicle control group was euthanized on Day 21 due to tumor progression. In contrast, all animals administered CD20 CAR T cells at all doses survived until the end of study (Day 62).

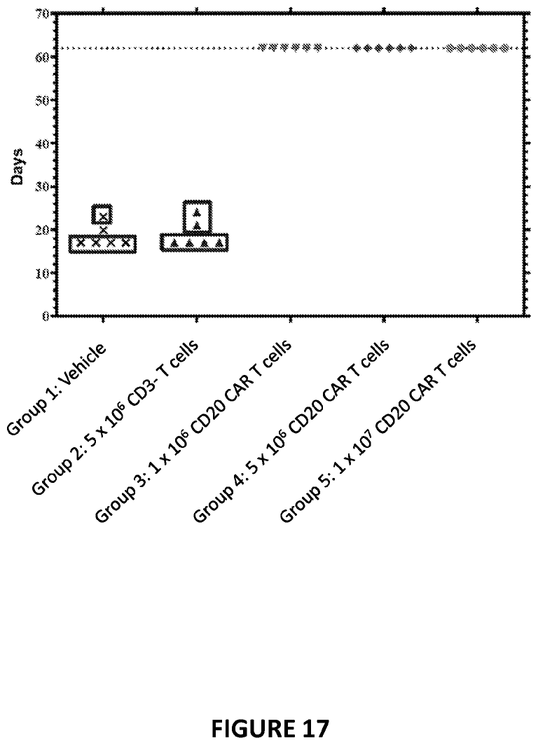

Results show an increase in TTE in CD20 CAR T cell-treated groups compared with vehicle control or CD3− control T cell groups. The median TTE for the vehicle control group and CD3− control T cell groups was 17 days, which established the maximum difference between CD20 CAR T cell treatment groups and vehicle control median TTEs (T−C) as 45 days in this 62-day study. Individual TTEs for all groups are shown in FIG. 17 and median TTEs are summarized in Table 6.

TABLE 6
Summary of response

| | | | | | | Statistical | | | | | | | |
|---|---|---|---|---|---|---|---|---|---|---|---|---|---|
| | | Dose | Median | | | Significance | | MTV (n) | Regressions | | | Deaths | |
| Group | Treatment | (cells/animal) | TTE | T-C | % TGD | vs G1 | vs G2 | Day 62 | PR | CR | TFS | TR | NTR |
| 1 | vehicle | NA | 17.0 | — | — | — | NS | — | 0 | 0 | 0 | 0 | 5 |
| 2 | CD3-T cells | 5 × 10⁶ | 17.0 | 0 | 0 | NS | — | — | 0 | 0 | 0 | 0 | 6 |
| 3 | CD20 CAR | 1 × 10⁶ | 62.0 | 45.0 | 265 | *** | *** | 0 (6) | 0 | 6 | 6 | 0 | 0 |
| | T low | | | | | | | | | | | | |
| 4 | CD20 CAR | 5 × 10⁶ | 62.0 | 45.0 | 265 | *** | *** | 0 (6) | 0 | 6 | 6 | 0 | 0 |
| | T mid | | | | | | | | | | | | |
| 5 | CD20 CAR | 1 × 10⁷ | 62.0 | 45.0 | 265 | *** | *** | 0 (6) | 0 | 6 | 6 | 0 | 0 |
| | T high | | | | | | | | | | | | |

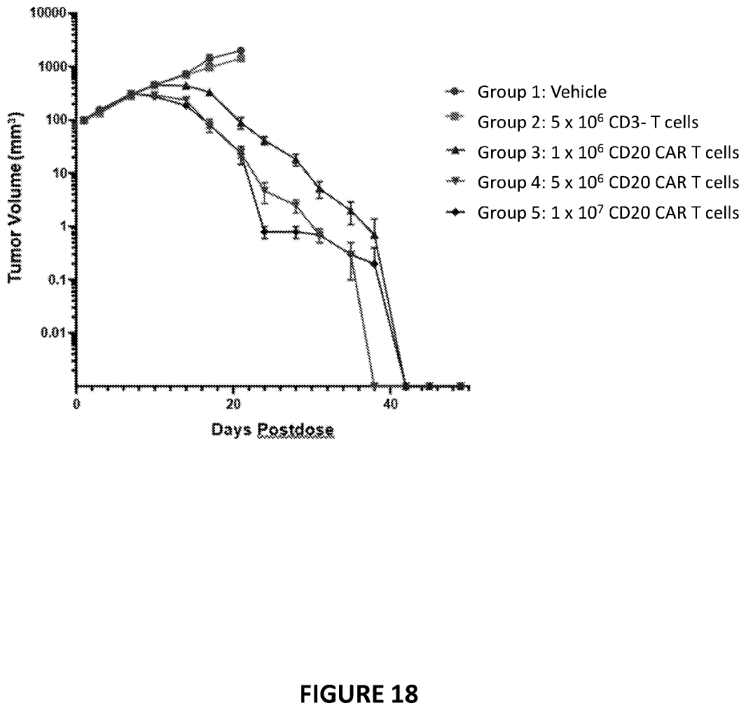

Tumor growth in the vehicle control and CD3− control T cell groups was progressive and similar between groups. Administration of CD20 CAR T cells resulted in dose-dependent reduction in tumor volume (FIG. 18). All animals administered CD20 CAR T cells experienced CR and were tumor free by Day 38 (5×10⁶ cells per animal) and Day 42 (1×10⁶ and 1×10⁷ cells per animal) (Table 6). All animals administered CD20 CAR T cells at all doses were assessed as tumor free survivors at the end of study (Day 62).

### Adverse Events

In Group 1, five NTR deaths due to tumor metastasis occurred on Day 17 (4 mice) and Day 23 (1 mouse). In Group 2, six NTR deaths due to tumor metastasis occurred on Day 17 (four mice), Day 21 (1 mouse), and Day 24 (1 mouse). Among the NTR deaths due to tumor metastasis, all mice that could be necropsied (n=8) displayed enlarged livers with numerous metastases.

Little or no group mean body weight loss occurred among all CD20 CAR T treatment groups.

### 4. Conclusions

This in vivo MCL xenograft study evaluated CD20 CAR T cells for antitumor efficacy in NSG mice bearing 16-day old subcutaneously implanted CD20+Granta-519 MCL tumors. Antitumor efficacy was evaluated by digital caliper measurements for calculation of tumor volume, body weight, and comparison of survival among vehicle control, CD3− control T cells, and CD20 CAR T cell treatment groups.

By Day 24 postdose, 5 out of 6 animals in the vehicle control group and all animals in the CD3− control group died due tumor metastasis. In contrast, all animals treated with CD20 CAR T cells (all doses) demonstrated complete tumor regression by Day 42, with tumors in all CD20 CAR T cell treated animals dropping below the limit of detection by caliper measurement. CD20 CAR T cells conferred significant survival advantages at all doses, with all mice administered CD20 CAR T cells remaining alive at Day 62, in comparison to animals administered CD3− control T cells.

These results demonstrate in vivo activity of CD20 CAR T cells of the invention against subcutaneous MCL tumors, including the ability of IV administered CD20 CAR T cells to traffic to distal tumor sites and mediate anti-tumor activity.

### Summary of Sequences

| NO: | Identifier | Sequence |
|---|---|---|
| 1 | muCD20 heavy | EVQLQQSGAELVKPGASVKMSCKASGYTFTSYNMHWVK |
| | chain variable | QTPGQGLEWIGAIYPGNGDTSYNQKFKGKATLTADKSSST |
| | (VH) region | AYMQLSSLTSEDSADYYCARSNYYGSSYWFFDVWGAGT |
| | | TVTVSS |
| 2 | muCD20 heavy | GAGGTGCAGCTGCAGCAGTCTGGGGCTGAGCTGGTGAA |
| | chain variable | GCCTGGGGCCTCAGTGAAGATGTCCTGCAAGGCTTCTG |
| | (VH) region | GCTACACATTTACCAGTTACAATATGCACTGGGTAAAG |
| | | CAGACACCTGGACAGGGCCTGGAATGGATTGGAGCTAT |
| | | TTATCCAGGAAATGGTGATACTTCCTACAATCAGAAGTT |
| | | CAAAGGCAAGGCCACATTGACTGCAGACAAATCCTCCA |
| | | GCACAGCCTACATGCAGCTCAGCAGCCTGACATCTGAG |
| | | GACTCTGCGGACTATTACTGTGCAAGATCTAATTATTAC |
| | | GGTAGTAGCTACTGGTTCTTCGATGTCTGGGGCGCAGG |
| | | GACCACGGTCACCGTCTCCTCA |
| 3 | muCD20 light | DIVLTQSPAILSASPGEKVTMTCRASSSVNYMDWYQ |
| | chain variable | KKPGSSPKPWIYATSNLASGVPARFSGSGSGTSYSL |
| | (VH) region | TISRVEAEDAATYYCQQWSFNPPTFGGGTKLEIK |
| 4 | muCD20 light | GACATTGTGCTGACCCAATCTCCAGCTATCCTGTCTGCA |
| | chain variable | TCTCCAGGGGAGAAGGTCACAATGACTTGCAGGGCCAG |
| | (VH) region | CTCAAGTGTAAATTACATGGACTGGTACCAGAAGAAGC |
| | | CAGGATCCTCCCCCAAACCCTGGATTTATGCCACATCCA |
| | | ACCTGGCTTCTGGAGTCCCTGCTCGCTTCAGTGGCAGTG |
| | | GGTCTGGGACCTCTTACTCTCTCACAATCAGCAGAGTGG |
| | | AGGCTGAAGATGCTGCCACTTATTACTGCCAGCAGTGG |
| | | AGTTTTAATCCACCCACGTTCGGAGGGGGGACCAAGCT |
| | | GGAAATAAAA |
| 5 | huCD20 heavy | EVQLVQSGAEVKKPGESLKISCKGSGYSFTSYWIGWVRQ |
| | chain variable | MPGKGLEWMGIIYPGDSDTRYSPSFQGQVTISADKSITT |
| | (VH) region | AYLQWSSLKASDTAMYYCARHPSYGSGSPNFDYWGQGTL |
| | | VTVSS |
| 6 | huCD20 heavy | GAAGTGCAGCTGGTCCAGTCTGGGGCCGAGGTGAAGAA |
| | chain variable | ACCTGGAGAAAGTCTGAAGATCTCATGTAAAGGCTCCG |
| | (VH) region | GGTACTCTTTCACAAGTTATTGGATTGGCTGGGTCCGAC |
| | | AGATGCCAGGAAAGGGCCTGGAGTGGATGGGAATCATC |
| | | TACCCCGGCGACAGCGATACCCGGTATTCTCCTAGTTTT |
| | | CAGGGCCAGGTGACAATCAGCGCAGACAAGTCCATTAC |
| | | CACAGCCTATCTGCAGTGGTCAAGCCTGAAAGCCTCTG |
| | | ATACCGCTATGTACTATTGTGCCAGGCACCCTAGCTACG |
| | | GGTCAGGAAGCCCAAACTTTGACTATTGGGGCCAGGGG |
| | | ACACTGGTGACTGTCTCCTCT |
| 7 | huCD20 light | DIVMTQTPLSSPVTLGQPASISCRSSQSLVYSDGNTYL |
| | chain variable | SWLQQRPGQPPRLLIYKISNRFSGVPDRFSGSGAGTDF |
| | (VH) region | TLKISRVEAEDVGVYYCVQATQFPLTFGGGTKVEIK |
| 8 | huCD20 light | GACATTGTGATGACTCAGACACCACTGAGCTCCCCAGT |
| | chain variable | GACTCTGGGACAGCCAGCCAGTATCTCATGCAGATCTA |
| | (VH) region | GTCAGTCACTGGTCTACAGCGACGGCAACACCTATCTG |
| | | AGCTGGCTGCAGCAGCGACCAGGACAGCCACCTAGACT |
| | | GCTGATCTACAAGATTTCCAATAGGTTCTCTGGAGTGCC |
| | | CGACCGCTTTAGCGGATCCGGAGCTGGAACTGATTTCA |
| | | CCCTGAAAATCTCCCGCGTGGAGGCTGAAGATGTGGGC |
| | | GTCTACTATTGCGTCCAGGCAACCCAGTTCCCTCTGACA |
| | | TTTGGCGGGGGAACTAAGGTGGAGATCAAG |
| 9 | muCD20 CDRH1 | SYNMH |
| 10 | muCD20 CDRH2 | AIYPGNGDTSYNQKFKG |
| 11 | muCD20 CDRH3 | SNYYGSSYWFFDV |
| 12 | muCD20 CDRL1 | RASSSVNYMD |
| 13 | muCD20 CDRL2 | ATSNLAS |
| 14 | muCD20 CDRL3 | QQWSFNPPT |
| 15 | huCD20 CDRH1 | SYWIG |
| 16 | huCD20 CDRH2 | IIYPGDSDTRYSPSFQG |
| 17 | huCD20 CDRH3 | HPSYGSGSPNFDY |
| 18 | huCD20 CDRL1 | RSSQSLVYSDGNTYLS |
| 19 | huCD20 CDRL2 | KISNRFS |
| 20 | huCD20 CDRL3 | VQATQFPLT |
| 21 | N1 co-stimulatory | KHSRKKFVHLLKRPFIKTTGAAQMEDASSCRCPQEEEGEC |
| | domain | DL |
| 22 | NI co-stimulatory | AAACATAGCCGCAAAAAATTTGTGCATCTGCTGAAACG |
| | domain | CCCGTTTATTAAAACCACCGGCGCGGCGCAGATGGAAG |
| | | ATGCGAGCAGCTGCCGCTGCCCGCAGGAAGAAGAAGG |
| | | CGAATGCGATCTG |
| 23 | N6 co-stimulatory | KASRKKAAAAAKSPFASPASSAQEEDASSCRAPSEEEGSC |
| | domain | EL |
| 24 | N6 co-stimulatory | AAAGCGAGCCGCAAAAAAGCGGCGGCGGCGGCGAAAA |
| | domain | GCCCGTTTGCGAGCCCGGCGAGCAGCGCGCAGGAAGAA |
| | | GATGCGAGCAGCTGCCGCGCGCCGAGCGAAGAAGAAG |
| | | GCAGCTGCGAACTG |
| 25 | Linker | GGGGSGGGGSGGGGS |
| 26 | Linker | GGAGGAGGAGGATCTGGAGGAGGAGGAAGTGGAGGAG |
| | | GAGGATCC |
| 27 | CD8 hinge | TTTPAPRPPTPAPTIASQPLSLRP |
| 28 | CD8 hinge | ACTACTACCCCAGCCCCACGTCCCCCCACGCCAGCTCCA |
| | | ACGATAGCAAGTCAGCCCTTATCTCTTCGCCCT |
| 29 | CD8 | EACRPAAGGAVHTRGLDFACDIYIWAPLAGTCGVLLLSLV |
| | transmembrane | FTLYC |
| 30 | CD8 | GAGGCTTGCAGGCCCGCGGCGGGCGGCGCCGTTCACAC |
| | transmembrane | GCGAGGACTAGACTTCGCCTGCGACATCTACATCTGGG |
| | | CACCACTAGCCGGGACTTGCGGAGTGTTGTTGTTGAGCT |
| | | TGGTAATAACGCTCTACTGC |
| 31 | CD3 zeta signaling | RVKFSRSADAPAYQQGQNQLYNELNLGRREEYDVLDKRR |
| | domain | GRDPEMGGKPRRKNPQEGLYNELQKDKMAEAYSEIGMK |
| | | GERRRGKGHDGLYQGLSTATKDTYDALHMQALPPR |
| 32 | CD3 zeta signaling | AGAGTGAAGTTCTCTCGCTCCGCGGACGCACCCGCTTA |
| | domain | CCAGCAGGGTCAGAACCAGCTATACAACGAGTTAAACC |
| | | TGGGGCGCCGGGAGGAGTACGACGTGTTAGACAAGCGT |
| | | AGAGGTAGGGACCCGGAGATGGGAGGCAAGCCTCGGA |
| | | GAAAGAACCCCCAGGAGGGCCTGTACAACGAACTCCAG |
| | | AAGGACAAGATGGCTGAGGCGTACTCGGAGATTGGTAT |
| | | GAAGGGCGAGAGACGTCGCGGAAAGGGACACGACGGC |
| | | TTATACCAGGGGCTTTCCACCGCGACCAAGGACACATA |
| | | CGACGCGCTGCACATGCAAGCCTTACCACCTCGA |
| 33 | CD8 signal peptide | MALPVTALLLPLALLLHAARP |
| 34 | CD8 signal peptide | ATGGCGCTCCCAGTGACAGCCTTACTTTTACCTCTGGCG |
| | | TTATTATTGCACGCGGCTCGTCCT |
| 35 | muCD20 scFv | DIVLTQSPAILSASPGEKVTMTCRASSSVNYMDWYQKKPG |
| | (VL-Linker-VH) | SSPKPWIYATSNLASGVPARFSGSGSGTSYSLTISRVEAE |
| | | DAATYYCQQWSFNPPTFGGGTKLEIKGSTSGGGSGGGSGG |
| | | GGSSEVQLQQSGAELVKPGASVKMSCKASGYTFTSYNMH |
| | | WVKQTPGQGLEWIGAIYPGNGDTSYNQKFKGKATLTADK |
| | | SSSTAYMQLSSLTSEDSADYYCARSNYYGSSYWFFDVWG |
| | | AGTTVTVSS |
| 36 | muCD20 scFv | GACATTGTGCTGACCCAATCTCCAGCTATCCTGTCTGCA |
| | (VL-Linker-VH) | TCTCCAGGGGAGAAGGTCACAATGACTTGCAGGGCCAG |
| | | CTCAAGTGTAAATTACATGGACTGGTACCAGAAGAAGC |
| | | CAGGATCCTCCCCCAAACCCTGGATTTATGCCACATCCA |
| | | ACCTGGCTTCTGGAGTCCCTGCTCGCTTCAGTGGCAGTG |
| | | GGTCTGGGACCTCTTACTCTCTCACAATCAGCAGAGTGG |
| | | AGGCTGAAGATGCTGCCACTTATTACTGCCAGCAGTGG |
| | | AGTTTTAATCCACCCACGTTCGGAGGGGGGACCAAGCT |
| | | GGAAATAAAAGGCAGTACTAGCGGTGGTGGCTCCGGGG |
| | | GCGGTTCCGGTGGGGGCGGCAGCAGCGAGGTGCAGCTG |
| | | CAGCAGTCTGGGGCTGAGCTGGTGAAGCCTGGGGCCTC |
| | | AGTGAAGATGTCCTGCAAGGCTTCTGGCTACACATTTAC |
| | | CAGTTACAATATGCACTGGGTAAAGCAGACACCTGGAC |
| | | AGGGCCTGGAATGGATTGGAGCTATTTATCCAGGAAAT |
| | | GGTGATACTTCCTACAATCAGAAGTTCAAAGGCAAGGC |
| | | CACATTGACTGCAGACAAATCCTCCAGCACAGCCTACA |
| | | TGCAGCTCAGCAGCCTGACATCTGAGGACTCTGCGGAC |
| | | TATTACTGTGCAAGATCTAATTATTACGGTAGTAGCTAC |
| | | TGGTTCTTCGATGTCTGGGGCGCAGGGACCACGGTCAC |
| | | CGTCTCCTCA |
| 37 | muCD20 scFv | EVQLQQSGAELVKPGASVKMSCKASGYTFTSYNMHWVK |
| | (VH-Linker-VL) | QTPGQGLEWIGAIYPGNGDTSYNQKFKGKATLTADKSSST |
| | | AYMQLSSLTSEDSADYYCARSNYYGSSYWFFDVWGAGT |
| | | TVTVSSGSTSGGGSGGGSGGGGSSDIVLTQSPAILSASPGE |
| | | KVTMTCRASSSVNYMDWYQKKPGSSPKPWIYATSNLASG |
| | | VPARFSGSGSGTSYSLTISRVEAEDAATYYCQQWSFNPPTF |
| | | GGGTKLEIK |
| 38 | muCD20 scFv | GAGGTGCAGCTGCAGCAGTCTGGGGCTGAGCTGGTGAA |
| | (VH-Linker-VL) | GCCTGGGGCCTCAGTGAAGATGTCCTGCAAGGCTTCTG |
| | | GCTACACATTTACCAGTTACAATATGCACTGGGTAAAG |
| | | CAGACACCTGGACAGGGCCTGGAATGGATTGGAGCTAT |
| | | TTATCCAGGAAATGGTGATACTTCCTACAATCAGAAGTT |
| | | CAAAGGCAAGGCCACATTGACTGCAGACAAATCCTCCA |
| | | GCACAGCCTACATGCAGCTCAGCAGCCTGACATCTGAG |
| | | GACTCTGCGGACTATTACTGTGCAAGATCTAATTATTAC |
| | | GGTAGTAGCTACTGGTTCTTCGATGTCTGGGGCGCAGG |
| | | GACCACGGTCACCGTCTCCTCAGGCAGTACTAGCGGTG |
| | | GTGGCTCCGGGGGCGGTTCCGGTGGGGGCGGCAGCAGC |
| | | GACATTGTGCTGACCCAATCTCCAGCTATCCTGTCTGCA |
| | | TCTCCAGGGGAGAAGGTCACAATGACTTGCAGGGCCAG |
| | | CTCAAGTGTAAATTACATGGACTGGTACCAGAAGAAGC |
| | | CAGGATCCTCCCCCAAACCCTGGATTTATGCCACATCCA |
| | | ACCTGGCTTCTGGAGTCCCTGCTCGCTTCAGTGGCAGTG |
| | | GGTCTGGGACCTCTTACTCTCTCACAATCAGCAGAGTGG |
| | | AGGCTGAAGATGCTGCCACTTATTACTGCCAGCAGTGG |
| | | AGTTTTAATCCACCCACGTTCGGAGGGGGGACCAAGCT |
| | | GGAAATAAAA |
| 39 | muCD20 scFv | DIVLTQSPAILSASPGEKVTMTCRASSSVNYMDWYQKKPG |
| | CAR (VL-Linker- | SSPKPWIYATSNLASGVPARFSGSGSGTSYSLTISRVEAE |
| | VH-CD8-CD8- | DAATYYCQQWSFNPPTFGGGTKLEIKGSTSGGGSGGGSGG |
| | N1-CD3) | GGSSEVQLQQSGAELVKPGASVKMSCKASGYTFTSYNMH |
| | | WVKQTPGQGLEWIGAIYPGNGDTSYNQKFKGKATLTADK |
| | | SSSTAYMQLSSLTSEDSADYYCARSNYYGSSYWFFDVWGA |
| | | GTTVTVSSTTTPAPRPPTPAPTIASQPLSLRPEACRPAAG |
| | | GAVHTRGLDFACDIYIWAPLAGTCGVLLLSLVITLYCKHS |
| | | RKKFVHLLKRPFIKTTGAAQMEDASSCRCPQEEEGECDLRV |
| | | KFSRSADAPAYQQGQNQLYNELNLGRREEYDVLDKRRGR |
| | | DPEMGGKPRRKNPQEGLYNELQKDKMAEAYSEIGMKGE |
| | | RRRGKGHDGLYQGLSTATKDTYDALHMQALPPR |
| 40 | muCD20 scFv | GACATTGTGCTGACCCAATCTCCAGCTATCCTGTCTGCA |
| | CAR (VL-Linker- | TCTCCAGGGGAGAAGGTCACAATGACTTGCAGGGCCAG |
| | VH-CD8-CD8- | CTCAAGTGTAAATTACATGGACTGGTACCAGAAGAAGC |
| | N1-CD3) | CAGGATCCTCCCCCAAACCCTGGATTTATGCCACATCCA |
| | | ACCTGGCTTCTGGAGTCCCTGCTCGCTTCAGTGGCAGTG |
| | | GGTCTGGGACCTCTTACTCTCTCACAATCAGCAGAGTGG |
| | | AGGCTGAAGATGCTGCCACTTATTACTGCCAGCAGTGG |
| | | AGTTTTAATCCACCCACGTTCGGAGGGGGGACCAAGCT |
| | | GGAAATAAAAGGCAGTACTAGCGGTGGTGGCTCCGGGG |
| | | GCGGTTCCGGTGGGGGCGGCAGCAGCGAGGTGCAGCTG |
| | | CAGCAGTCTGGGGCTGAGCTGGTGAAGCCTGGGGCCTC |
| | | AGTGAAGATGTCCTGCAAGGCTTCTGGCTACACATTTAC |
| | | CAGTTACAATATGCACTGGGTAAAGCAGACACCTGGAC |
| | | AGGGCCTGGAATGGATTGGAGCTATTTATCCAGGAAAT |
| | | GGTGATACTTCCTACAATCAGAAGTTCAAAGGCAAGGC |
| | | CACATTGACTGCAGACAAATCCTCCAGCACAGCCTACA |
| | | TGCAGCTCAGCAGCCTGACATCTGAGGACTCTGCGGAC |
| | | TATTACTGTGCAAGATCTAATTATTACGGTAGTAGCTAC |
| | | TGGTTCTTCGATGTCTGGGGCGCAGGGACCACGGTCAC |
| | | CGTCTCCTCAACTACTACCCCAGCCCCACGTCCCCCCAC |
| | | GCCAGCTCCAACGATAGCAAGTCAGCCCTTATCTCTTCG |
| | | CCCTGAGGCTTGCAGGCCCGCGGCGGGCGGCGCCGTTC |
| | | ACACGCGAGGACTAGACTTCGCCTGCGACATCTACATC |
| | | TGGGCACCACTAGCCGGGACTTGCGGAGTGTTGTTGTT |
| | | GAGCTTGGTAATAACGCTCTACTGCAAACATAGCCGCA |
| | | AAAAATTTGTGCATCTGCTGAAACGCCCGTTTATTAAAA |
| | | CCACCGGCGCGGCGCAGATGGAAGATGCGAGCAGCTGC |
| | | CGCTGCCCGCAGGAAGAAGAAGGCGAATGCGATCTGA |
| | | GAGTGAAGTTCTCTCGCTCCGCGGACGCACCCGCTTACC |
| | | AGCAGGGTCAGAACCAGCTATACAACGAGTTAAACCTG |
| | | GGGCGCCGGGAGGAGTACGACGTGTTAGACAAGCGTA |
| | | GAGGTAGGGACCCGGAGATGGGAGGCAAGCCTCGGAG |
| | | AAAGAACCCCCAGGAGGGCCTGTACAACGAACTCCAGA |
| | | AGGACAAGATGGCTGAGGCGTACTCGGAGATTGGTATG |
| | | AAGGGCGAGAGACGTCGCGGAAAGGGACACGACGGCT |
| | | TATACCAGGGGCTTTCCACCGCGACCAAGGACACATAC |
| | | GACGCGCTGCACATGCAAGCCTTACCACCTCGA |
| 41 | muCD20 scFv | EVQLQQSGAELVKPGASVKMSCKASGYTFTSYNMHWVK |
| | CAR (VH-Linker- | QTPGQGLEWIGAIYPGNGDTSYNQKFKGKATLTADKSSST |
| | VL-CD8-CD8-N1- | AYMQLSSLTSEDSADYYCARSNYYGSSYWFFDVWGAGT |
| | CD3) | TVTVSSGSTSGGGSGGGSGGGGSSDIVLTQSPAILSASPGE |
| | | KVTMTCRASSSVNYMDWYQKKPGSSPKPWIYATSNLASG |
| | | VPARFSGSGSGTSYSLTISRVEAEDAATYYCQQWSFNPPTF |
| | | GGGTKLEIKTTTPAPRPPTPAPTIASQPLSLRPEACRPAAGG |
| | | AVHTRGLDFACDIYIWAPLAGTCGVLLLSLVITLYCKHSR |
| | | KKFVHLLKRPFIKTTGAAQMEDASSCRCPQEEEGECDLRV |
| | | KFSRSADAPAYQQGQNQLYNELNLGRREEYDVLDKRRGR |
| | | DPEMGGKPRRKNPQEGLYNELQKDKMAEAYSEIGMKGE |
| | | RRRGKGHDGLYQGLSTATKDTYDALHMQALPPR |
| 42 | muCD20 scFv | GAGGTGCAGCTGCAGCAGTCTGGGGCTGAGCTGGTGAA |
| | CAR (VH-Linker- | GCCTGGGGCCTCAGTGAAGATGTCCTGCAAGGCTTCTG |
| | VL-CD8-CD8-N1- | GCTACACATTTACCAGTTACAATATGCACTGGGTAAAG |
| | CD3) | CAGACACCTGGACAGGGCCTGGAATGGATTGGAGCTAT |
| | | TTATCCAGGAAATGGTGATACTTCCTACAATCAGAAGTT |
| | | CAAAGGCAAGGCCACATTGACTGCAGACAAATCCTCCA |
| | | GCACAGCCTACATGCAGCTCAGCAGCCTGACATCTGAG |
| | | GACTCTGCGGACTATTACTGTGCAAGATCTAATTATTAC |
| | | GGTAGTAGCTACTGGTTCTTCGATGTCTGGGGCGCAGG |
| | | GACCACGGTCACCGTCTCCTCAGGCAGTACTAGCGGTG |
| | | GTGGCTCCGGGGGCGGTTCCGGTGGGGGCGGCAGCAGC |
| | | GACATTGTGCTGACCCAATCTCCAGCTATCCTGTCTGCA |
| | | TCTCCAGGGGAGAAGGTCACAATGACTTGCAGGGCCAG |
| | | CTCAAGTGTAAATTACATGGACTGGTACCAGAAGAAGC |
| | | CAGGATCCTCCCCCAAACCCTGGATTTATGCCACATCCA |
| | | ACCTGGCTTCTGGAGTCCCTGCTCGCTTCAGTGGCAGTG |
| | | GGTCTGGGACCTCTTACTCTCTCACAATCAGCAGAGTGG |
| | | AGGCTGAAGATGCTGCCACTTATTACTGCCAGCAGTGG |
| | | AGTTTTAATCCACCCACGTTCGGAGGGGGGACCAAGCT |
| | | GGAAATAAAAACTACTACCCCAGCCCCACGTCCCCCCA |
| | | CGCCAGCTCCAACGATAGCAAGTCAGCCCTTATCTCTTC |
| | | GCCCTGAGGCTTGCAGGCCCGCGGCGGGCGGCGCCGTT |
| | | CACACGCGAGGACTAGACTTCGCCTGCGACATCTACAT |
| | | CTGGGCACCACTAGCCGGGACTTGCGGAGTGTTGTTGTT |
| | | GAGCTTGGTAATAACGCTCTACTGCAAACATAGCCGCA |
| | | AAAAATTTGTGCATCTGCTGAAACGCCCGTTTATTAAAA |
| | | CCACCGGCGCGGCGCAGATGGAAGATGCGAGCAGCTGC |
| | | CGCTGCCCGCAGGAAGAAGAAGGCGAATGCGATCTGA |
| | | GAGTGAAGTTCTCTCGCTCCGCGGACGCACCCGCTTACC |
| | | AGCAGGGTCAGAACCAGCTATACAACGAGTTAAACCTG |
| | | GGGCGCCGGGAGGAGTACGACGTGTTAGACAAGCGTA |
| | | GAGGTAGGGACCCGGAGATGGGAGGCAAGCCTCGGAG |
| | | AAAGAACCCCCAGGAGGGCCTGTACAACGAACTCCAGA |
| | | AGGACAAGATGGCTGAGGCGTACTCGGAGATTGGTATG |
| | | AAGGGCGAGAGACGTCGCGGAAAGGGACACGACGGCT |
| | | TATACCAGGGGCTTTCCACCGCGACCAAGGACACATAC |
| | | GACGCGCTGCACATGCAAGCCTTACCACCTCGA |
| 43 | muCD20 scFv | DIVLTQSPAILSASPGEKVTMTCRASSSVNYMDWYQKKPG |
| | CAR (VL-Linker- | SSPKPWIYATSNLASGVPARFSGSGSGTSYSLTISRVEAED |
| | VH-CD8-CD8- | AATYYCQQWSFNPPTFGGGTKLEIKGSTSGGGSGGGSGG |
| | N6-CD3) | GGSSEVQLQQSGAELVKPGASVKMSCKASGYTFTSYNMH |
| | | WVKQTPGQGLEWIGAIYPGNGDTSYNQKFKGKATLTADK |
| | | SSSTAYMQLSSLTSEDSADYYCARSNYYGSSYWFFDVWGA |
| | | AGTTVTVSSTTTPAPRPPTPAPTIASQPLSLRPEACRPAAGG |
| | | VHTRGLDFACDIYIWAPLAGTCGVLLLSLVITLYCKASR |
| | | KKAAAAAKSPFASPASSAQEEDASSCRAPSEEEGSCELRV |
| | | KFSRSADAPAYQQGQNQLYNELNLGRREEYDVLDKRRGR |
| | | DPEMGGKPRRKNPQEGLYNELQKDKMAEAYSEIGMKGE |
| | | RRRGKGHDGLYQGLSTATKDTYDALHMQALPPR |
| 44 | muCD20 scFv | GACATTGTGCTGACCCAATCTCCAGCTATCCTGTCTGCA |
| | CAR (VL-Linker- | TCTCCAGGGGAGAAGGTCACAATGACTTGCAGGGCCAG |
| | VH-CD8-CD8- | CTCAAGTGTAAATTACATGGACTGGTACCAGAAGAAGC |
| | N6-CD3) | CAGGATCCTCCCCCAAACCCTGGATTTATGCCACATCCA |
| | | ACCTGGCTTCTGGAGTCCCTGCTCGCTTCAGTGGCAGTG |
| | | GGTCTGGGACCTCTTACTCTCTCACAATCAGCAGAGTGG |
| | | AGGCTGAAGATGCTGCCACTTATTACTGCCAGCAGTGG |
| | | AGTTTTAATCCACCCACGTTCGGAGGGGGGACCAAGCT |
| | | GGAAATAAAAGGCAGTACTAGCGGTGGTGGCTCCGGGG |
| | | GCGGTTCCGGTGGGGGCGGCAGCAGCGAGGTGCAGCTG |
| | | CAGCAGTCTGGGGCTGAGCTGGTGAAGCCTGGGGCCTC |
| | | AGTGAAGATGTCCTGCAAGGCTTCTGGCTACACATTTAC |
| | | CAGTTACAATATGCACTGGGTAAAGCAGACACCTGGAC |
| | | AGGGCCTGGAATGGATTGGAGCTATTTATCCAGGAAAT |
| | | GGTGATACTTCCTACAATCAGAAGTTCAAAGGCAAGGC |
| | | CACATTGACTGCAGACAAATCCTCCAGCACAGCCTACA |
| | | TGCAGCTCAGCAGCCTGACATCTGAGGACTCTGCGGAC |
| | | TATTACTGTGCAAGATCTAATTATTACGGTAGTAGCTAC |
| | | TGGTTCTTCGATGTCTGGGGCGCAGGGACCACGGTCAC |
| | | CGTCTCCTCAACTACTACCCCAGCCCCACGTCCCCCCAC |
| | | GCCAGCTCCAACGATAGCAAGTCAGCCCTTATCTCTTCG |
| | | CCCTGAGGCTTGCAGGCCCGCGGCGGGCGGCGCCGTTC |
| | | ACACGCGAGGACTAGACTTCGCCTGCGACATCTACATC |
| | | TGGGCACCACTAGCCGGGACTTGCGGAGTGTTGTTGTT |
| | | GAGCTTGGTAATAACGCTCTACTGCAAAGCGAGCCGCA |
| | | AAAAAGCGGCGGCGGCGGCGAAAAGCCCGTTTGCGAG |
| | | CCCGGCGAGCAGCGCGCAGGAAGAAGATGCGAGCAGC |
| | | TGCCGCGCGCCGAGCGAAGAAGAAGGCAGCTGCGAAC |
| | | TGAGAGTGAAGTTCTCTCGCTCCGCGGACGCACCCGCTT |
| | | ACCAGCAGGGTCAGAACCAGCTATACAACGAGTTAAAC |
| | | CTGGGGCGCCGGGAGGAGTACGACGTGTTAGACAAGCG |
| | | TAGAGGTAGGGACCCGGAGATGGGAGGCAAGCCTCGG |
| | | AGAAAGAACCCCCAGGAGGGCCTGTACAACGAACTCCA |
| | | GAAGGACAAGATGGCTGAGGCGTACTCGGAGATTGGTA |
| | | TGAAGGGCGAGAGACGTCGCGGAAAGGGACACGACGG |
| | | CTTATACCAGGGGCTTTCCACCGCGACCAAGGACACAT |
| | | ACGACGCGCTGCACATGCAAGCCTTACCACCTCGA |
| 45 | muCD20 scFv | EVQLQQSGAELVKPGASVKMSCKASGYTFTSYNMHWVK |
| | CAR (VH-Linker- | QTPGQGLEWIGAIYPGNGDTSYNQKFKGKATLTADKSSST |
| | VL-CD8-CD8-N6- | AYMQLSSLTSEDSADYYCARSNYYGSSYWFFDVWGAGT |
| | CD3) | TVTVSSGSTSGGGSGGGSGGGGSSDIVLTQSPAILSASPGE |
| | | KVTMTCRASSSVNYMDWYQKKPGSSPKPWIYATSNLASG |
| | | VPARFSGSGSGTSYSLTISRVEAEDAATYYCQQWSFNPPTF |
| | | GGGTKLEIKTTTPAPRPPTPAPTIASQPLSLRPEACRPAAGG |
| | | AVHTRGLDFACDIYIWAPLAGTCGVLLLSLVITLYCKASR |
| | | KKAAAAAKSPFASPASSAQEEDASSCRAPSEEEGSCELRV |
| | | KFSRSADAPAYQQGQNQLYNELNLGRREEYDVLDKRRGR |
| | | DPEMGGKPRRKNPQEGLYNELQKDKMAEAYSEIGMKGE |
| | | RRRGKGHDGLYQGLSTATKDTYDALHMQALPPR |
| 46 | muCD20 scFv | GAGGTGCAGCTGCAGCAGTCTGGGGCTGAGCTGGTGAA |
| | CAR (VH-Linker- | GCCTGGGGCCTCAGTGAAGATGTCCTGCAAGGCTTCTG |
| | VL-CD8-CD8-N6- | GCTACACATTTACCAGTTACAATATGCACTGGGTAAAG |
| | CD3) | CAGACACCTGGACAGGGCCTGGAATGGATTGGAGCTAT |
| | | TTATCCAGGAAATGGTGATACTTCCTACAATCAGAAGTT |
| | | CAAAGGCAAGGCCACATTGACTGCAGACAAATCCTCCA |
| | | GCACAGCCTACATGCAGCTCAGCAGCCTGACATCTGAG |
| | | GACTCTGCGGACTATTACTGTGCAAGATCTAATTATTAC |
| | | GGTAGTAGCTACTGGTTCTTCGATGTCTGGGGCGCAGG |
| | | GACCACGGTCACCGTCTCCTCAGGCAGTACTAGCGGTG |
| | | GTGGCTCCGGGGGCGGTTCCGGTGGGGGCGGCAGCAGC |
| | | GACATTGTGCTGACCCAATCTCCAGCTATCCTGTCTGCA |
| | | TCTCCAGGGGAGAAGGTCACAATGACTTGCAGGGCCAG |
| | | CTCAAGTGTAAATTACATGGACTGGTACCAGAAGAAGC |
| | | CAGGATCCTCCCCCAAACCCTGGATTTATGCCACATCCA |
| | | ACCTGGCTTCTGGAGTCCCTGCTCGCTTCAGTGGCAGTG |
| | | GGTCTGGGACCTCTTACTCTCTCACAATCAGCAGAGTGG |
| | | AGGCTGAAGATGCTGCCACTTATTACTGCCAGCAGTGG |
| | | AGTTTTAATCCACCCACGTTCGGAGGGGGGACCAAGCT |
| | | GGAAATAAAAACTACTACCCCAGCCCCACGTCCCCCCA |
| | | CGCCAGCTCCAACGATAGCAAGTCAGCCCTTATCTCTTC |
| | | GCCCTGAGGCTTGCAGGCCCGCGGCGGGCGGCGCCGTT |
| | | CACACGCGAGGACTAGACTTCGCCTGCGACATCTACAT |
| | | CTGGGCACCACTAGCCGGGACTTGCGGAGTGTTGTTGTT |
| | | GAGCTTGGTAATAACGCTCTACTGCAAAGCGAGCCGCA |
| | | AAAAAGCGGCGGCGGCGGCGAAAAGCCCGTTTGCGAG |
| | | CCCGGCGAGCAGCGCGCAGGAAGAAGATGCGAGCAGC |
| | | TGCCGCGCGCCGAGCGAAGAAGAAGGCAGCTGCGAAC |
| | | TGAGAGTGAAGTTCTCTCGCTCCGCGGACGCACCCGCTT |
| | | ACCAGCAGGGTCAGAACCAGCTATACAACGAGTTAAAC |
| | | CTGGGGCGCCGGGAGGAGTACGACGTGTTAGACAAGCG |
| | | TAGAGGTAGGGACCCGGAGATGGGAGGCAAGCCTCGG |
| | | AGAAAGAACCCCCAGGAGGGCCTGTACAACGAACTCCA |
| | | GAAGGACAAGATGGCTGAGGCGTACTCGGAGATTGGTA |
| | | TGAAGGGCGAGAGACGTCGCGGAAAGGGACACGACGG |
| | | CTTATACCAGGGGCTTTCCACCGCGACCAAGGACACAT |
| | | ACGACGCGCTGCACATGCAAGCCTTACCACCTCGA |
| 47 | huCD20 scFv | DIVMTQTPLSSPVTLGQPASISCRSSQSLVYSDGNTYLSWL |
| | (VL-Linker-VH) | QQRPGQPPRLLIYKISNRFSGVPDRFSGSGAGTDFTLKISRV |
| | | EAEDVGVYYCVQATQFPLTFGGGTKVEIKGGGGSGGGGS |
| | | GGGGSEVQLVQSGAEVKKPGESLKISCKGSGYSFTSYWIG |
| | | WVRQMPGKGLEWMGIIYPGDSDTRYSPSFQGQVTISADKS |
| | | ITTAYLQWSSLKASDTAMYYCARHPSYGSGSPNFDYWGQ |
| | | GTLVTVSS |
| 48 | huCD20 scFv | GACATTGTGATGACTCAGACACCACTGAGCTCCCCAGT |
| | (VL-Linker-VH) | GACTCTGGGACAGCCAGCCAGTATCTCATGCAGATCTA |
| | | GTCAGTCACTGGTCTACAGCGACGGCAACACCTATCTG |
| | | AGCTGGCTGCAGCAGCGACCAGGACAGCCACCTAGACT |
| | | GCTGATCTACAAGATTTCCAATAGGTTCTCTGGAGTGCC |
| | | CGACCGCTTTAGCGGATCCGGAGCTGGAACTGATTTCA |
| | | CCCTGAAAATCTCCCGCGTGGAGGCTGAAGATGTGGGC |
| | | GTCTACTATTGCGTCCAGGCAACCCAGTTCCCTCTGACA |
| | | TTTGGCGGGGGAACTAAGGTGGAGATCAAGGGAGGAG |
| | | GAGGATCTGGAGGAGGAGGAAGTGGAGGAGGAGGATC |
| | | CGAAGTGCAGCTGGTCCAGTCTGGGGCCGAGGTGAAGA |
| | | AACCTGGAGAAAGTCTGAAGATCTCATGTAAAGGCTCC |
| | | GGGTACTCTTTCACAAGTTATTGGATTGGCTGGGTCCGA |
| | | CAGATGCCAGGAAAGGGCCTGGAGTGGATGGGAATCAT |
| | | CTACCCCGGCGACAGCGATACCCGGTATTCTCCTAGTTT |
| | | TCAGGGCCAGGTGACAATCAGCGCAGACAAGTCCATTA |
| | | CCACAGCCTATCTGCAGTGGTCAAGCCTGAAAGCCTCT |
| | | GATACCGCTATGTACTATTGTGCCAGGCACCCTAGCTAC |
| | | GGGTCAGGAAGCCCAAACTTTGACTATTGGGGCCAGGG |
| | | GACACTGGTGACTGTCTCCTCT |
| 49 | huCD20 scFv | EVQLVQSGAEVKKPGESLKISCKGSGYSFTSYWIGWVRQ |
| | (VH-Linker-VL) | MPGKGLEWMGIIYPGDSDTRYSPSFQGQVTISADKSITTAY |
| | | LQWSSLKASDTAMYYCARHPSYGSGSPNFDYWGQGTLV |
| | | TVSSGGGGSGGGGSGGGGSDIVMTQTPLSSPVTLGQPASIS |
| | | CRSSQSLVYSDGNTYLSWLQQRPGQPPRLLIYKISNRFSGV |
| | | PDRFSGSGAGTDFTLKISRVEAEDVGVYYCVQATQFPLTF |
| | | GGGTKVEIK |
| 50 | huCD20 scFv | GAAGTGCAGCTGGTCCAGTCTGGGGCCGAGGTGAAGAA |
| | (VH-Linker-VL) | ACCTGGAGAAAGTCTGAAGATCTCATGTAAAGGCTCCG |
| | | GGTACTCTTTCACAAGTTATTGGATTGGCTGGGTCCGAC |
| | | AGATGCCAGGAAAGGGCCTGGAGTGGATGGGAATCATC |
| | | TACCCCGGCGACAGCGATACCCGGTATTCTCCTAGTTTT |
| | | CAGGGCCAGGTGACAATCAGCGCAGACAAGTCCATTAC |
| | | CACAGCCTATCTGCAGTGGTCAAGCCTGAAAGCCTCTG |
| | | ATACCGCTATGTACTATTGTGCCAGGCACCCTAGCTACG |
| | | GGTCAGGAAGCCCAAACTTTGACTATTGGGGCCAGGGG |
| | | ACACTGGTGACTGTCTCCTCTGGAGGAGGAGGATCTGG |
| | | AGGAGGAGGAAGTGGAGGAGGAGGATCCGACATTGTG |
| | | ATGACTCAGACACCACTGAGCTCCCCAGTGACTCTGGG |
| | | ACAGCCAGCCAGTATCTCATGCAGATCTAGTCAGTCAC |
| | | TGGTCTACAGCGACGGCAACACCTATCTGAGCTGGCTG |
| | | CAGCAGCGACCAGGACAGCCACCTAGACTGCTGATCTA |
| | | CAAGATTTCCAATAGGTTCTCTGGAGTGCCCGACCGCTT |
| | | TAGCGGATCCGGAGCTGGAACTGATTTCACCCTGAAAA |
| | | TCTCCCGCGTGGAGGCTGAAGATGTGGGCGTCTACTATT |
| | | GCGTCCAGGCAACCCAGTTCCCTCTGACATTTGGCGGG |
| | | GGAACTAAGGTGGAGATCAAG |
| 51 | huCD20 scFv | DIVMTQTPLSSPVTLGQPASISCRSSQSLVYSDGNTYLSWL |
| | CAR (VL-Linker- | QQRPGQPPRLLIYKISNRFSGVPDRFSGSGAGTDFTLKISRV |
| | VH-CD8-CD8- | EAEDVGVYYCVQATQFPLTFGGGTKVEIKGGGGSGGGGS |
| | N1-CD3) | GGGGSEVQLVQSGAEVKKPGESLKISCKGSGYSFTSYWIG |
| | | WVRQMPGKGLEWMGIIYPGDSDTRYSPSFQGQVTISADKS |
| | | ITTAYLQWSSLKASDTAMYYCARHPSYGSGSPNFDYWGQ |
| | | GTLVTVSSTTTPAPRPPTPAPTIASQPLSLRPEACRPAAGGA |
| | | VHTRGLDFACDIYIWAPLAGTCGVLLLSLVITLYCKHSRK |
| | | KFVHLLKRPFIKTTGAAQMEDASSCRCPQEEEGECDLRVK |
| | | FSRSADAPAYQQGQNQLYNELNLGRREEYDVLDKRRGRD |
| | | PEMGGKPRRKNPQEGLYNELQKDKMAEAYSEIGMKGER |
| | | RRGKGHDGLYQGLSTATKDTYDALHMQALPPR |
| 52 | huCD20 scFv | GACATTGTGATGACTCAGACACCACTGAGCTCCCCAGT |
| | CAR (VL-Linker- | GACTCTGGGACAGCCAGCCAGTATCTCATGCAGATCTA |
| | VH-CD8-CD8- | GTCAGTCACTGGTCTACAGCGACGGCAACACCTATCTG |
| | N1-CD3) | AGCTGGCTGCAGCAGCGACCAGGACAGCCACCTAGACT |
| | | GCTGATCTACAAGATTTCCAATAGGTTCTCTGGAGTGCC |
| | | CGACCGCTTTAGCGGATCCGGAGCTGGAACTGATTTCA |
| | | CCCTGAAAATCTCCCGCGTGGAGGCTGAAGATGTGGGC |
| | | GTCTACTATTGCGTCCAGGCAACCCAGTTCCCTCTGACA |
| | | TTTGGCGGGGGAACTAAGGTGGAGATCAAGGGAGGAG |
| | | GAGGATCTGGAGGAGGAGGAAGTGGAGGAGGAGGATC |
| | | CGAAGTGCAGCTGGTCCAGTCTGGGGCCGAGGTGAAGA |
| | | AACCTGGAGAAAGTCTGAAGATCTCATGTAAAGGCTCC |
| | | GGGTACTCTTTCACAAGTTATTGGATTGGCTGGGTCCGA |
| | | CAGATGCCAGGAAAGGGCCTGGAGTGGATGGGAATCAT |
| | | CTACCCCGGCGACAGCGATACCCGGTATTCTCCTAGTTT |
| | | TCAGGGCCAGGTGACAATCAGCGCAGACAAGTCCATTA |
| | | CCACAGCCTATCTGCAGTGGTCAAGCCTGAAAGCCTCT |
| | | GATACCGCTATGTACTATTGTGCCAGGCACCCTAGCTAC |
| | | GGGTCAGGAAGCCCAAACTTTGACTATTGGGGCCAGGG |
| | | GACACTGGTGACTGTCTCCTCTACTACTACCCCAGCCCC |
| | | ACGTCCCCCCACGCCAGCTCCAACGATAGCAAGTCAGC |
| | | CCTTATCTCTTCGCCCTGAGGCTTGCAGGCCCGCGGCGG |
| | | GCGGCGCCGTTCACACGCGAGGACTAGACTTCGCCTGC |
| | | GACATCTACATCTGGGCACCACTAGCCGGGACTTGCGG |
| | | AGTGTTGTTGTTGAGCTTGGTAATAACGCTCTACTGCAA |
| | | ACATAGCCGCAAAAAATTTGTGCATCTGCTGAAACGCC |
| | | CGTTTATTAAAACCACCGGCGCGGCGCAGATGGAAGAT |
| | | GCGAGCAGCTGCCGCTGCCCGCAGGAAGAAGAAGGCG |
| | | AATGCGATCTGAGAGTGAAGTTCTCTCGCTCCGCGGAC |
| | | GCACCCGCTTACCAGCAGGGTCAGAACCAGCTATACAA |
| | | CGAGTTAAACCTGGGGCGCCGGGAGGAGTACGACGTGT |
| | | TAGACAAGCGTAGAGGTAGGGACCCGGAGATGGGAGG |
| | | CAAGCCTCGGAGAAAGAACCCCCAGGAGGGCCTGTACA |
| | | ACGAACTCCAGAAGGACAAGATGGCTGAGGCGTACTCG |
| | | GAGATTGGTATGAAGGGCGAGAGACGTCGCGGAAAGG |
| | | GACACGACGGCTTATACCAGGGGCTTTCCACCGCGACC |
| | | AAGGACACATACGACGCGCTGCACATGCAAGCCTTACC |
| | | ACCTCGA |
| 53 | huCD20 scFv | EVQLVQSGAEVKKPGESLKISCKGSGYSFTSYWIGWVRQ |
| | CAR (VH-Linker- | MPGKGLEWMGIIYPGDSDTRYSPSFQGQVTISADKSITTAY |
| | VL-CD8-CD8-N1- | LQWSSLKASDTAMYYCARHPSYGSGSPNFDYWGQGTLV |
| | CD3) | TVSSGGGGSGGGGSGGGGSDIVMTQTPLSSPVTLGQPASIS |
| | | CRSSQSLVYSDGNTYLSWLQQRPGQPPRLLIYKISNRFSGV |
| | | PDRFSGSGAGTDFTLKISRVEAEDVGVYYCVQATQFPLTF |
| | | GGGTKVEIKTTTPAPRPPTPAPTIASQPLSLRPEACRPAAGG |
| | | AVHTRGLDFACDIYIWAPLAGTCGVLLLSLVITLYCKHSR |
| | | KKFVHLLKRPFIKTTGAAQMEDASSCRCPQEEEGECDLRV |
| | | KFSRSADAPAYQQGQNQLYNELNLGRREEYDVLDKRRGR |
| | | DPEMGGKPRRKNPQEGLYNELQKDKMAEAYSEIGMKGE |
| | | RRRGKGHDGLYQGLSTATKDTYDALHMQALPPR |
| 54 | huCD20 scFv | GAAGTGCAGCTGGTCCAGTCTGGGGCCGAGGTGAAGAA |
| | CAR (VH-Linker- | ACCTGGAGAAAGTCTGAAGATCTCATGTAAAGGCTCCG |
| | VL-CD8-CD8-N1- | GGTACTCTTTCACAAGTTATTGGATTGGCTGGGTCCGAC |
| | CD3) | AGATGCCAGGAAAGGGCCTGGAGTGGATGGGAATCATC |
| | | TACCCCGGCGACAGCGATACCCGGTATTCTCCTAGTTTT |
| | | CAGGGCCAGGTGACAATCAGCGCAGACAAGTCCATTAC |
| | | CACAGCCTATCTGCAGTGGTCAAGCCTGAAAGCCTCTG |
| | | ATACCGCTATGTACTATTGTGCCAGGCACCCTAGCTACG |
| | | GGTCAGGAAGCCCAAACTTTGACTATTGGGGCCAGGGG |
| | | ACACTGGTGACTGTCTCCTCTGGAGGAGGAGGATCTGG |
| | | AGGAGGAGGAAGTGGAGGAGGAGGATCCGACATTGTG |
| | | ATGACTCAGACACCACTGAGCTCCCCAGTGACTCTGGG |
| | | ACAGCCAGCCAGTATCTCATGCAGATCTAGTCAGTCAC |
| | | TGGTCTACAGCGACGGCAACACCTATCTGAGCTGGCTG |
| | | CAGCAGCGACCAGGACAGCCACCTAGACTGCTGATCTA |
| | | CAAGATTTCCAATAGGTTCTCTGGAGTGCCCGACCGCTT |
| | | TAGCGGATCCGGAGCTGGAACTGATTTCACCCTGAAAA |
| | | TCTCCCGCGTGGAGGCTGAAGATGTGGGCGTCTACTATT |
| | | GCGTCCAGGCAACCCAGTTCCCTCTGACATTTGGCGGG |
| | | GGAACTAAGGTGGAGATCAAGACTACTACCCCAGCCCC |
| | | ACGTCCCCCCACGCCAGCTCCAACGATAGCAAGTCAGC |
| | | CCTTATCTCTTCGCCCTGAGGCTTGCAGGCCCGCGGCGG |
| | | GCGGCGCCGTTCACACGCGAGGACTAGACTTCGCCTGC |
| | | GACATCTACATCTGGGCACCACTAGCCGGGACTTGCGG |
| | | AGTGTTGTTGTTGAGCTTGGTAATAACGCTCTACTGCAA |
| | | ACATAGCCGCAAAAAATTTGTGCATCTGCTGAAACGCC |
| | | CGTTTATTAAAACCACCGGCGCGGCGCAGATGGAAGAT |
| | | GCGAGCAGCTGCCGCTGCCCGCAGGAAGAAGAAGGCG |
| | | AATGCGATCTGAGAGTGAAGTTCTCTCGCTCCGCGGAC |
| | | GCACCCGCTTACCAGCAGGGTCAGAACCAGCTATACAA |
| | | CGAGTTAAACCTGGGGCGCCGGGAGGAGTACGACGTGT |
| | | TAGACAAGCGTAGAGGTAGGGACCCGGAGATGGGAGG |
| | | CAAGCCTCGGAGAAAGAACCCCCAGGAGGGCCTGTACA |
| | | ACGAACTCCAGAAGGACAAGATGGCTGAGGCGTACTCG |
| | | GAGATTGGTATGAAGGGCGAGAGACGTCGCGGAAAGG |
| | | GACACGACGGCTTATACCAGGGGCTTTCCACCGCGACC |
| | | AAGGACACATACGACGCGCTGCACATGCAAGCCTTACC |
| | | ACCTCGA |
| 55 | huCD20 scFv | DIVMTQTPLSSPVTLGQPASISCRSSQSLVYSDGNTYLSWL |
| | CAR (VL-Linker- | QQRPGQPPRLLIYKISNRFSGVPDRFSGSGAGTDFTLKISRV |
| | VH-CD8-CD8- | EAEDVGVYYCVQATQFPLTFGGGTKVEIKGGGGSGGGGS |
| | N6-CD3) | GGGGSEVQLVQSGAEVKKPGESLKISCKGSGYSFTSYWIG |
| | | WVRQMPGKGLEWMGIIYPGDSDTRYSPSFQGQVTISADKS |
| | | ITTAYLQWSSLKASDTAMYYCARHPSYGSGSPNFDYWGQ |
| | | GTLVTVSSTTTPAPRPPTPAPTIASQPLSLRPEACRPAAGGA |
| | | VHTRGLDFACDIYIWAPLAGTCGVLLLSLVITLYCKASRK |
| | | KAAAAAKSPFASPASSAQEEDASSCRAPSEEEGSCELRVKF |
| | | SRSADAPAYQQGQNQLYNELNLGRREEYDVLDKRRGRDP |
| | | EMGGKPRRKNPQEGLYNELQKDKMAEAYSEIGMKGERR |
| | | RGKGHDGLYQGLSTATKDTYDALHMQALPPR |
| 56 | huCD20 scFv | GACATTGTGATGACTCAGACACCACTGAGCTCCCCAGT |
| | CAR (VL-Linker- | GACTCTGGGACAGCCAGCCAGTATCTCATGCAGATCTA |
| | VH-CD8-CD8- | GTCAGTCACTGGTCTACAGCGACGGCAACACCTATCTG |
| | N6-CD3) | AGCTGGCTGCAGCAGCGACCAGGACAGCCACCTAGACT |
| | | GCTGATCTACAAGATTTCCAATAGGTTCTCTGGAGTGCC |
| | | CGACCGCTTTAGCGGATCCGGAGCTGGAACTGATTTCA |
| | | CCCTGAAAATCTCCCGCGTGGAGGCTGAAGATGTGGGC |
| | | GTCTACTATTGCGTCCAGGCAACCCAGTTCCCTCTGACA |
| | | TTTGGCGGGGGAACTAAGGTGGAGATCAAGGGAGGAG |
| | | GAGGATCTGGAGGAGGAGGAAGTGGAGGAGGAGGATC |
| | | CGAAGTGCAGCTGGTCCAGTCTGGGGCCGAGGTGAAGA |
| | | AACCTGGAGAAAGTCTGAAGATCTCATGTAAAGGCTCC |
| | | GGGTACTCTTTCACAAGTTATTGGATTGGCTGGGTCCGA |
| | | CAGATGCCAGGAAAGGGCCTGGAGTGGATGGGAATCAT |
| | | CTACCCCGGCGACAGCGATACCCGGTATTCTCCTAGTTT |
| | | TCAGGGCCAGGTGACAATCAGCGCAGACAAGTCCATTA |
| | | CCACAGCCTATCTGCAGTGGTCAAGCCTGAAAGCCTCT |
| | | GATACCGCTATGTACTATTGTGCCAGGCACCCTAGCTAC |
| | | GGGTCAGGAAGCCCAAACTTTGACTATTGGGGCCAGGG |
| | | GACACTGGTGACTGTCTCCTCTACTACTACCCCAGCCCC |
| | | ACGTCCCCCCACGCCAGCTCCAACGATAGCAAGTCAGC |
| | | CCTTATCTCTTCGCCCTGAGGCTTGCAGGCCCGCGGCGG |
| | | GCGGCGCCGTTCACACGCGAGGACTAGACTTCGCCTGC |
| | | GACATCTACATCTGGGCACCACTAGCCGGGACTTGCGG |
| | | AGTGTTGTTGTTGAGCTTGGTAATAACGCTCTACTGCAA |
| | | AGCGAGCCGCAAAAAAGCGGCGGCGGCGGCGAAAAGC |
| | | CCGTTTGCGAGCCCGGCGAGCAGCGCGCAGGAAGAAG |
| | | ATGCGAGCAGCTGCCGCGCGCCGAGCGAAGAAGAAGG |
| | | CAGCTGCGAACTGAGAGTGAAGTTCTCTCGCTCCGCGG |
| | | ACGCACCCGCTTACCAGCAGGGTCAGAACCAGCTATAC |
| | | AACGAGTTAAACCTGGGGCGCCGGGAGGAGTACGACGT |
| | | GTTAGACAAGCGTAGAGGTAGGGACCCGGAGATGGGA |
| | | GGCAAGCCTCGGAGAAAGAACCCCCAGGAGGGCCTGT |
| | | ACAACGAACTCCAGAAGGACAAGATGGCTGAGGCGTA |
| | | CTCGGAGATTGGTATGAAGGGCGAGAGACGTCGCGGAA |
| | | AGGGACACGACGGCTTATACCAGGGGCTTTCCACCGCG |
| | | ACCAAGGACACATACGACGCGCTGCACATGCAAGCCTT |
| | | ACCACCTCGA |
| 57 | huCD20 scFv | EVQLVQSGAEVKKPGESLKISCKGSGYSFTSYWIGWVRQ |
| | CAR (VH-Linker- | MPGKGLEWMGIIYPGDSDTRYSPSFQGQVTISADKSITTAY |
| | VL-CD8-CD8-N6- | LQWSSLKASDTAMYYCARHPSYGSGSPNFDYWGQGTLV |
| | CD3) | TVSSGGGGSGGGGSGGGGSDIVMTQTPLSSPVTLGQPASIS |
| | | CRSSQSLVYSDGNTYLSWLQQRPGQPPRLLIYKISNRFSGV |
| | | PDRFSGSGAGTDFTLKISRVEAEDVGVYYCVQATQFPLTF |
| | | GGGTKVEIKTTTPAPRPPTPAPTIASQPLSLRPEACRPAAGG |
| | | AVHTRGLDFACDIYIWAPLAGTCGVLLLSLVITLYCKASR |
| | | KKAAAAAKSPFASPASSAQEEDASSCRAPSEEEGSCELRV |
| | | KFSRSADAPAYQQGQNQLYNELNLGRREEYDVLDKRRGR |
| | | DPEMGGKPRRKNPQEGLYNELQKDKMAEAYSEIGMKGE |
| | | RRRGKGHDGLYQGLSTATKDTYDALHMQALPPR |
| 58 | huCD20 scFv | GAAGTGCAGCTGGTCCAGTCTGGGGCCGAGGTGAAGAA |
| | CAR (VH-Linker- | ACCTGGAGAAAGTCTGAAGATCTCATGTAAAGGCTCCG |
| | VL-CD8-CD8-N6- | GGTACTCTTTCACAAGTTATTGGATTGGCTGGGTCCGAC |
| | CD3) | AGATGCCAGGAAAGGGCCTGGAGTGGATGGGAATCATC |
| | | TACCCCGGCGACAGCGATACCCGGTATTCTCCTAGTTTT |
| | | CAGGGCCAGGTGACAATCAGCGCAGACAAGTCCATTAC |
| | | CACAGCCTATCTGCAGTGGTCAAGCCTGAAAGCCTCTG |
| | | ATACCGCTATGTACTATTGTGCCAGGCACCCTAGCTACG |
| | | GGTCAGGAAGCCCAAACTTTGACTATTGGGGCCAGGGG |
| | | ACACTGGTGACTGTCTCCTCTGGAGGAGGAGGATCTGG |
| | | AGGAGGAGGAAGTGGAGGAGGAGGATCCGACATTGTG |
| | | ATGACTCAGACACCACTGAGCTCCCCAGTGACTCTGGG |
| | | ACAGCCAGCCAGTATCTCATGCAGATCTAGTCAGTCAC |
| | | TGGTCTACAGCGACGGCAACACCTATCTGAGCTGGCTG |
| | | CAGCAGCGACCAGGACAGCCACCTAGACTGCTGATCTA |
| | | CAAGATTTCCAATAGGTTCTCTGGAGTGCCCGACCGCTT |
| | | TAGCGGATCCGGAGCTGGAACTGATTTCACCCTGAAAA |
| | | TCTCCCGCGTGGAGGCTGAAGATGTGGGCGTCTACTATT |
| | | GCGTCCAGGCAACCCAGTTCCCTCTGACATTTGGCGGG |
| | | GGAACTAAGGTGGAGATCAAGACTACTACCCCAGCCCC |
| | | ACGTCCCCCCACGCCAGCTCCAACGATAGCAAGTCAGC |
| | | CCTTATCTCTTCGCCCTGAGGCTTGCAGGCCCGCGGCGG |
| | | GCGGCGCCGTTCACACGCGAGGACTAGACTTCGCCTGC |
| | | GACATCTACATCTGGGCACCACTAGCCGGGACTTGCGG |
| | | AGTGTTGTTGTTGAGCTTGGTAATAACGCTCTACTGCAA |
| | | AGCGAGCCGCAAAAAAGCGGCGGCGGCGGCGAAAAGC |
| | | CCGTTTGCGAGCCCGGCGAGCAGCGCGCAGGAAGAAG |
| | | ATGCGAGCAGCTGCCGCGCGCCGAGCGAAGAAGAAGG |
| | | CAGCTGCGAACTGAGAGTGAAGTTCTCTCGCTCCGCGG |
| | | ACGCACCCGCTTACCAGCAGGGTCAGAACCAGCTATAC |
| | | AACGAGTTAAACCTGGGGCGCCGGGAGGAGTACGACGT |
| | | GTTAGACAAGCGTAGAGGTAGGGACCCGGAGATGGGA |
| | | GGCAAGCCTCGGAGAAAGAACCCCCAGGAGGGCCTGT |
| | | ACAACGAACTCCAGAAGGACAAGATGGCTGAGGCGTA |
| | | CTCGGAGATTGGTATGAAGGGCGAGAGACGTCGCGGAA |
| | | AGGGACACGACGGCTTATACCAGGGGCTTTCCACCGCG |
| | | ACCAAGGACACATACGACGCGCTGCACATGCAAGCCTT |
| | | ACCACCTCGA |
| 59 | CD8 hinge region | AKPTTTPAPRPPTPAPTIASQPLSLRPEACRPAAGGAVHTR |
| | | GLDFA |
| 60 | CD28 hinge region | KIEVMYPPPYLDNEKSNGTIIHVKGKHLCPSPLFPGPSKP |
| 61 | CD8-CD28 hinge | AKPTTTPAPRPPTPAPTIASQPLSLRPEACRPAAGGAVHTR |
| | region | GLDFAPRKIEVMYPPPYLDNEKSNGTIIHVKGKHLCPSPLF |
| | | PGPSKP |
| 62 | CD3 | LDPKLCYLLDGILFIYGVILTALFLRVK |
| | transmembrane | |
| | domain | |
| 63 | CD3 y | LCYLLDGILFIYGVILTALFL |
| | transmembrane | |
| | domain | |
| 64 | CD28 | FWVLVVVGGVLACYSLLVTVAFIIFWV |
| | transmembrane | |
| | domain | |
| 65 | Human CD20 | MTTPRNSVNGTFPAEPMKGPIAMQSGPKPLFRRMSSLVGP |
| | | TQSFFMRESKTLGAVQIMNGLFHIALGGLLMIPAGIYAPIC |
| | | VTVWYPLWGGIMYIISGSLLAATEKNSRKCLVKGKMIMN |
| | | SLSLFAAISGMILSIMDILNIKISHFLKMESLNFIRAHTPYINI |
| | | YNCEPANPSEKNSPSTQYCYSIQSLFLGILSVMLIFAFFQEL |
| | | VIAGIVENEWKRTCSRPKSNIVLLSAEEKKEQTIEIKEEVVG |
| | | LTETSSQPKNEEDIEIIPIQEEEEEETETNFPEPPQDQESSPIE |
| | | NDSSP |
| 66 | TRC 1-2 | TGGCCTGGAGCAACAAATCTGA |
| | recognition | |
| | sequence (sense) | |
| 67 | TRC 1-2 | ACCGGACCTCGTTGTTTAGACT |
| | recognition | |
| | sequence | |
| | (antisense) | |
| 68 | TRC 1-2L.1592 | MNTKYNKEFLLYLAGFVDGDGSIYAVIYPHQRAKFKHFL |
| | meganuclease | KLLFTVSQSTKRRWFLDKLVDEIGVGYVYDLPRTSEYRLS |
| | | EIKPLHNFLTQLQPFLKLKQKQANLVLKIIEQLPSAKESPD |
| | | KFLEVCTWVDQIAALNDSRTRKTTSETVRAVLDSLPGSVG |
| | | GLSPSQASSAASSASSSPGSGISEALRAGAGSGTGYNKEFL |
| | | LYLAGFVDGDGSIYACIRPRQGSKFKHRLTLGFAVGQKTQ |
| | | RRWFLDKLVDEIGVGYVYDRGSVSEYVLSEIKPLHNFLTQ |
| | | LQPFLKLKQKQANLVLKIIEQLPSAKESPDKFLEVCTWVD |
| | | QIAALNDSKTRKTTSETVRAVLDSLSEKKKSSP |
| 69 | TRC 1-2L.1775 | MNTKYNKEFLLYLAGFVDGDGSIYACIYPHQRAKFKHLL |
| | meganuclease | KLVFAVHQRTTRRWFLDKLVDEIGVGYVYDIGSVSEYRLS |
| | | QIKPLHNFLTQLQPFLKLKQKQANLVLKIIEQLPSAKESPD |
| | | KFLEVCTWVDQIAALNDSRTRKTTSETVRAVLDSLPGSVG |
| | | GLSPSQASSAASSASSSPGSGISEALRAGAGSGTGYNKEFL |
| | | LYLAGFVDGDGSIYACIAPRQGSKFKHRLKLGFAVGQKTQ |
| | | RRWFLDKLVDEIGVGYVYDRGSVSEYVLSEIKPLHNFLTQ |
| | | LQPFLKLKQKQANLVLKIIEQLPSAKESPDKFLEVCTWVD |
| | | QIAALNDSKTRKTTSETVRAVLDSLSEKKKSSP |
| 70 | TRC l-2x.87EE | MNTKYNKEFLLYLAGFVDGDGSIFASIYPHQRAKFKHFLK |
| | meganuclease | LTFAVYQKTQRRWFLDKLVDEIGVGYVYDSGSVSEYRLS |
| | | EIKPLHNFLTQLQPFLKLKQKQANLVLKIIEQLPSAKESPD |
| | | KFLEVCTWVDQIAALNDSRTRKTTSETVRAVLDSLPGSVG |
| | | GLSPSQASSAASSASSSPGSGISEALRAGAGSGTGYNKEFL |
| | | LYLAGFVDGDGSIYACIAPRQGSKFKHRLKLGFAVGQKTQ |
| | | RRWFLDKLVDEIGVGYVYDRGSVSEYVLSEIKPLHNFLTQ |
| | | LQPFLKLKQKQANLVLKIIEQLPSAKESPDKFLEVCTWVD |
| | | QIAALNDSKTRKTTSETVRAVLDSLSEKKKSSP |
| 71 | Linker | GSTSGGGSGGGSGGGGSS |
| 72 | Linker | GGCAGTACTAGCGGTGGTGGCTCCGGGGGCGGTTCCGG |
| | | TGGGGGCGGCAGCAGC |
| 73 | muCD20 scFv | MALPVTALLLPLALLLHAARPDIVLTQSPAILSASPGEKVT |
| | CAR (CD8sp-VL- | MTCRASSSVNYMDWYQKKPGSSPKPWIYATSNLASGVPA |
| | Linker-VH-CD8- | RFSGSGSGTSYSLTISRVEAEDAATYYCQQWSFNPPTFGG |
| | CD8-N6-CD3) | GTKLEIKGSTSGGGSGGGSGGGGSSEVQLQQSGAELVKPG |
| | | ASVKMSCKASGYTFTSYNMHWVKQTPGQGLEWIGAIYPG |
| | | NGDTSYNQKFKGKATLTADKSSSTAYMQLSSLTSEDSAD |
| | | YYCARSNYYGSSYWFFDVWGAGTTVTVSSTTTPAPRPPTP |
| | | APTIASQPLSLRPEACRPAAGGAVHTRGLDFACDIYIWAPL |
| | | AGTCGVLLLSLVITLYCKASRKKAAAAAKSPFASPASSAQ |
| | | EEDASSCRAPSEEEGSCELRVKFSRSADAPAYQQGQNQLY |
| | | NELNLGRREEYDVLDKRRGRDPEMGGKPRRKNPQEGLYN |
| | | ELQKDKMAEAYSEIGMKGERRRGKGHDGLYQGLSTATK |
| | | DTYDALHMQALPPR |
| 74 | muCD20 scFv | ATGGCGCTCCCAGTGACAGCCTTACTTTTACCTCTGGCG |
| | CAR (CD8sp-VL- | TTATTATTGCACGCGGCTCGTCCTGACATTGTGCTGACC |
| | Linker-VH-CD8- | CAATCTCCAGCTATCCTGTCTGCATCTCCAGGGGAGAA |
| | CD8-N6-CD3) | GGTCACAATGACTTGCAGGGCCAGCTCAAGTGTAAATT |
| | | ACATGGACTGGTACCAGAAGAAGCCAGGATCCTCCCCC |
| | | AAACCCTGGATTTATGCCACATCCAACCTGGCTTCTGGA |
| | | GTCCCTGCTCGCTTCAGTGGCAGTGGGTCTGGGACCTCT |
| | | TACTCTCTCACAATCAGCAGAGTGGAGGCTGAAGATGC |
| | | TGCCACTTATTACTGCCAGCAGTGGAGTTTTAATCCACC |
| | | CACGTTCGGAGGGGGGACCAAGCTGGAAATAAAAGGC |
| | | AGTACTAGCGGTGGTGGCTCCGGGGGCGGTTCCGGTGG |
| | | GGGCGGCAGCAGCGAGGTGCAGCTGCAGCAGTCTGGG |
| | | GCTGAGCTGGTGAAGCCTGGGGCCTCAGTGAAGATGTC |
| | | CTGCAAGGCTTCTGGCTACACATTTACCAGTTACAATAT |
| | | GCACTGGGTAAAGCAGACACCTGGACAGGGCCTGGAAT |
| | | GGATTGGAGCTATTTATCCAGGAAATGGTGATACTTCCT |
| | | ACAATCAGAAGTTCAAAGGCAAGGCCACATTGACTGCA |
| | | GACAAATCCTCCAGCACAGCCTACATGCAGCTCAGCAG |
| | | CCTGACATCTGAGGACTCTGCGGACTATTACTGTGCAA |
| | | GATCTAATTATTACGGTAGTAGCTACTGGTTCTTCGATG |
| | | TCTGGGGCGCAGGGACCACGGTCACCGTCTCCTCAACT |
| | | ACTACCCCAGCCCCACGTCCCCCCACGCCAGCTCCAAC |
| | | GATAGCAAGTCAGCCCTTATCTCTTCGCCCTGAGGCTTG |
| | | CAGGCCCGCGGCGGGCGGCGCCGTTCACACGCGAGGAC |
| | | TAGACTTCGCCTGCGACATCTACATCTGGGCACCACTAG |
| | | CCGGGACTTGCGGAGTGTTGTTGTTGAGCTTGGTAATAA |
| | | CGCTCTACTGCAAAGCGAGCCGCAAAAAAGCGGCGGCG |
| | | GCGGCGAAAAGCCCGTTTGCGAGCCCGGCGAGCAGCGC |
| | | GCAGGAAGAAGATGCGAGCAGCTGCCGCGCGCCGAGC |
| | | GAAGAAGAAGGCAGCTGCGAACTGAGAGTGAAGTTCTC |
| | | TCGCTCCGCGGACGCACCCGCTTACCAGCAGGGTCAGA |
| | | ACCAGCTATACAACGAGTTAAACCTGGGGCGCCGGGAG |
| | | GAGTACGACGTGTTAGACAAGCGTAGAGGTAGGGACCC |
| | | GGAGATGGGAGGCAAGCCTCGGAGAAAGAACCCCCAG |
| | | GAGGGCCTGTACAACGAACTCCAGAAGGACAAGATGG |
| | | CTGAGGCGTACTCGGAGATTGGTATGAAGGGCGAGAGA |
| | | CGTCGCGGAAAGGGACACGACGGCTTATACCAGGGGCT |
| | | TTCCACCGCGACCAAGGACACATACGACGCGCTGCACA |
| | | TGCAAGCCTTACCACCTCGATGA |
| 75 | huCD20 scFv | MALPVTALLLPLALLLHAARPDIVMTQTPLSSPVTLGQPAS |
| | CAR (CD8sp-VL- | ISCRSSQSLVYSDGNTYLSWLQQRPGQPPRLLIYKISNRFSG |
| | Linker-VH-CD8- | VPDRFSGSGAGTDFTLKISRVEAEDVGVYYCVQATQFPLT |
| | CD8-N6-CD3) | FGGGTKVEIKGGGGSGGGGSGGGGSEVQLVQSGAEVKKP |
| | | GESLKISCKGSGYSFTSYWIGWVRQMPGKGLEWMGIIYPG |
| | | DSDTRYSPSFQGQVTISADKSETTAYLQWSSLKASDTAMY |
| | | YCARHPSYGSGSPNFDYWGQGTLVTVSSTTTPAPRPPTPA |
| | | PTIASQPLSLRPEACRPAAGGAVHTRGLDFACDIYIWAPLA |
| | | GTCGVLLLSLVITLYCKASRKKAAAAAKSPFASPASSAQE |
| | | EDASSCRAPSEEEGSCELRVKFSRSADAPAYQQGQNQLYN |
| | | ELNLGRREEYDVLDKRRGRDPEMGGKPRRKNPQEGLYNE |
| | | LQKDKMAEAYSEIGMKGERRRGKGHDGLYQGLSTATKD |
| | | TYDALHMQALPPR |
| 76 | huCD20 scFv | ATGGCGCTCCCAGTGACAGCCTTACTTTTACCTCTGGCG |
| | CAR (CD8sp-VL- | TTATTATTGCACGCGGCTCGTCCTGACATTGTGATGACT |
| | Linker-VH-CD8- | CAGACACCACTGAGCTCCCCAGTGACTCTGGGACAGCC |
| | CD8-N6-CD3) | AGCCAGTATCTCATGCAGATCTAGTCAGTCACTGGTCTA |
| | | CAGCGACGGCAACACCTATCTGAGCTGGCTGCAGCAGC |
| | | GACCAGGACAGCCACCTAGACTGCTGATCTACAAGATT |
| | | TCCAATAGGTTCTCTGGAGTGCCCGACCGCTTTAGCGGA |
| | | TCCGGAGCTGGAACTGATTTCACCCTGAAAATCTCCCGC |
| | | GTGGAGGCTGAAGATGTGGGCGTCTACTATTGCGTCCA |
| | | GGCAACCCAGTTCCCTCTGACATTTGGCGGGGGAACTA |
| | | AGGTGGAGATCAAGGGAGGAGGAGGATCTGGAGGAGG |
| | | AGGAAGTGGAGGAGGAGGATCCGAAGTGCAGCTGGTC |
| | | CAGTCTGGGGCCGAGGTGAAGAAACCTGGAGAAAGTCT |
| | | GAAGATCTCATGTAAAGGCTCCGGGTACTCTTTCACAA |
| | | GTTATTGGATTGGCTGGGTCCGACAGATGCCAGGAAAG |
| | | GGCCTGGAGTGGATGGGAATCATCTACCCCGGCGACAG |
| | | CGATACCCGGTATTCTCCTAGTTTTCAGGGCCAGGTGAC |
| | | AATCAGCGCAGACAAGTCCATTACCACAGCCTATCTGC |
| | | AGTGGTCAAGCCTGAAAGCCTCTGATACCGCTATGTAC |
| | | TATTGTGCCAGGCACCCTAGCTACGGGTCAGGAAGCCC |
| | | AAACTTTGACTATTGGGGCCAGGGGACACTGGTGACTG |
| | | TCTCCTCTACTACTACCCCAGCCCCACGTCCCCCCACGC |
| | | CAGCTCCAACGATAGCAAGTCAGCCCTTATCTCTTCGCC |
| | | CTGAGGCTTGCAGGCCCGCGGCGGGCGGCGCCGTTCAC |
| | | ACGCGAGGACTAGACTTCGCCTGCGACATCTACATCTG |
| | | GGCACCACTAGCCGGGACTTGCGGAGTGTTGTTGTTGA |
| | | GCTTGGTAATAACGCTCTACTGCAAAGCGAGCCGCAAA |
| | | AAAGCGGCGGCGGCGGCGAAAAGCCCGTTTGCGAGCCC |
| | | GGCGAGCAGCGCGCAGGAAGAAGATGCGAGCAGCTGC |
| | | CGCGCGCCGAGCGAAGAAGAAGGCAGCTGCGAACTGA |
| | | GAGTGAAGTTCTCTCGCTCCGCGGACGCACCCGCTTACC |
| | | AGCAGGGTCAGAACCAGCTATACAACGAGTTAAACCTG |
| | | GGGCGCCGGGAGGAGTACGACGTGTTAGACAAGCGTA |
| | | GAGGTAGGGACCCGGAGATGGGAGGCAAGCCTCGGAG |
| | | AAAGAACCCCCAGGAGGGCCTGTACAACGAACTCCAGA |
| | | AGGACAAGATGGCTGAGGCGTACTCGGAGATTGGTATG |
| | | AAGGGCGAGAGACGTCGCGGAAAGGGACACGACGGCT |
| | | TATACCAGGGGCTTTCCACCGCGACCAAGGACACATAC |
| | | GACGCGCTGCACATGCAAGCCTTACCACCTCGATGA |
| 77 | huCD20 scFv | MALPVTALLLPLALLLHAARPDIVMTQTPLSSPVTLGQPAS |
| | CAR (CD8sp-VL- | ISCRSSQSLVYSDGNTYLSWLQQRPGQPPRLLIYKISNRFSG |
| | Linker-VH-CD8- | VPDRFSGSGAGTDFTLKISRVEAEDVGVYYCVQATQFPLT |
| | CD8-41BB-CD3) | FGGGTKVEIKGGGGSGGGGSGGGGSEVQLVQSGAEVKKP |
| | | GESLKISCKGSGYSFTSYWIGWVRQMPGKGLEWMGIIYPG |
| | | DSDTRYSPSFQGQVTISADKSETTAYLQWSSLKASDTAMY |
| | | YCARHPSYGSGSPNFDYWGQGTLVTVSSTTTPAPRPPTPA |
| | | PTIASQPLSLRPEACRPAAGGAVHTRGLDFACDIYIWAPLA |
| | | GTCGVLLLSLVITLYCKRGRKKLLYIFKQPFMRPVQTTQEE |
| | | DGCSCRFPEEEEGGCELRVKFSRSADAPAYQQGQNQLYN |
| | | ELNLGRREEYDVLDKRRGRDPEMGGKPRRKNPQEGLYNE |
| | | LQKDKMAEAYSEIGMKGERRRGKGHDGLYQGLSTATKD |
| | | TYDALHMQALPPR |
| 78 | huCD20 scFv | ATGGCGCTCCCAGTGACAGCCTTACTTTTACCTCTGGCG |
| | CAR (CD8sp-VL- | TTATTATTGCACGCGGCTCGTCCTGACATTGTGATGACT |
| | Linker-VH-CD8- | CAGACACCACTGAGCTCCCCAGTGACTCTGGGACAGCC |
| | CD8-41BB-CD3) | AGCCAGTATCTCATGCAGATCTAGTCAGTCACTGGTCTA |
| | | CAGCGACGGCAACACCTATCTGAGCTGGCTGCAGCAGC |
| | | GACCAGGACAGCCACCTAGACTGCTGATCTACAAGATT |
| | | TCCAATAGGTTCTCTGGAGTGCCCGACCGCTTTAGCGGA |
| | | TCCGGAGCTGGAACTGATTTCACCCTGAAAATCTCCCGC |
| | | GTGGAGGCTGAAGATGTGGGCGTCTACTATTGCGTCCA |
| | | GGCAACCCAGTTCCCTCTGACATTTGGCGGGGGAACTA |
| | | AGGTGGAGATCAAGGGAGGAGGAGGATCTGGAGGAGG |
| | | AGGAAGTGGAGGAGGAGGATCCGAAGTGCAGCTGGTC |
| | | CAGTCTGGGGCCGAGGTGAAGAAACCTGGAGAAAGTCT |
| | | GAAGATCTCATGTAAAGGCTCCGGGTACTCTTTCACAA |
| | | GTTATTGGATTGGCTGGGTCCGACAGATGCCAGGAAAG |
| | | GGCCTGGAGTGGATGGGAATCATCTACCCCGGCGACAG |
| | | CGATACCCGGTATTCTCCTAGTTTTCAGGGCCAGGTGAC |
| | | AATCAGCGCAGACAAGTCCATTACCACAGCCTATCTGC |
| | | AGTGGTCAAGCCTGAAAGCCTCTGATACCGCTATGTAC |
| | | TATTGTGCCAGGCACCCTAGCTACGGGTCAGGAAGCCC |
| | | AAACTTTGACTATTGGGGCCAGGGGACACTGGTGACTG |
| | | TCTCCTCTACTACTACCCCAGCCCCACGTCCCCCCACGC |
| | | CAGCTCCAACGATAGCAAGTCAGCCCTTATCTCTTCGCC |
| | | CTGAGGCTTGCAGGCCCGCGGCGGGCGGCGCCGTTCAC |
| | | ACGCGAGGACTAGACTTCGCCTGCGACATCTACATCTG |
| | | GGCACCACTAGCCGGGACTTGCGGAGTGTTGTTGTTGA |
| | | GCTTGGTAATAACGCTCTACTGCAAGCGTGGGAGAAAG |
| | | AAGCTCTTGTACATTTTCAAGCAGCCATTCATGCGTCCC |
| | | GTTCAGACGACTCAGGAGGAGGACGGCTGCTCGTGCCG |
| | | ATTCCCGGAGGAGGAGGAGGGCGGTTGCGAACTCAGA |
| | | GTGAAGTTCTCTCGCTCCGCGGACGCACCCGCTTACCAG |
| | | CAGGGTCAGAACCAGCTATACAACGAGTTAAACCTGGG |
| | | GCGCCGGGAGGAGTACGACGTGTTAGACAAGCGTAGA |
| | | GGTAGGGACCCGGAGATGGGAGGCAAGCCTCGGAGAA |
| | | AGAACCCCCAGGAGGGCCTGTACAACGAACTCCAGAAG |
| | | GACAAGATGGCTGAGGCGTACTCGGAGATTGGTATGAA |
| | | GGGCGAGAGACGTCGCGGAAAGGGACACGACGGCTTA |
| | | TACCAGGGGCTTTCCACCGCGACCAAGGACACATACGA |
| | | CGCGCTGCACATGCAAGCCTTACCACCTCGATGA |
| 79 | huCD20 scFv | MALPVTALLLPLALLLHAARPDIVMTQTPLSSPVTLGQPAS |
| | CAR (CD8sp-VL- | ISCRSSQSLVYSDGNTYLSWLQQRPGQPPRLLIYKISNRFSG |
| | Linker-VH-CD8- | VPDRFSGSGAGTDFTLKISRVEAEDVGVYYCVQATQFPLT |
| | CD8-41BBmDel- | FGGGTKVEIKGGGGSGGGGSGGGGSEVQLVQSGAEVKKP |
| | CD3) | GESLKISCKGSGYSFTSYWIGWVRQMPGKGLEWMGIIYPG |
| | | DSDTRYSPSFQGQVTISADKSETTAYLQWSSLKASDTAMY |
| | | YCARHPSYGSGSPNFDYWGQGTLVTVSSTTTPAPRPPTPA |
| | | PTIASQPLSLRPEACRPAAGGAVHTRGLDFACDIYIWAPLA |
| | | GTCGVLLLSLVITLYCKRGSSELLYIFKQPFMRPVQTTSQQ |
| | | NGCSCEFPQQQQGGCELRVKFSRSADAPAYQQGQNQLYN |
| | | ELNLGRREEYDVLDKRRGRDPEMGGKPRRKNPQEGLYNE |
| | | LQKDKMAEAYSEIGMKGERRRGKGHDGLYQGLSTATKD |
| | | TYDALHMQALPPR |
| 80 | huCD20 scFv | ATGGCGCTCCCAGTGACAGCCTTACTTTTACCTCTGGCG |
| | CAR (CD8sp-VL- | TTATTATTGCACGCGGCTCGTCCTGACATTGTGATGACT |
| | Linker-VH-CD8- | CAGACACCACTGAGCTCCCCAGTGACTCTGGGACAGCC |
| | CD8-41BBmDel- | AGCCAGTATCTCATGCAGATCTAGTCAGTCACTGGTCTA |
| | CD3) | CAGCGACGGCAACACCTATCTGAGCTGGCTGCAGCAGC |
| | | GACCAGGACAGCCACCTAGACTGCTGATCTACAAGATT |
| | | TCCAATAGGTTCTCTGGAGTGCCCGACCGCTTTAGCGGA |
| | | TCCGGAGCTGGAACTGATTTCACCCTGAAAATCTCCCGC |
| | | GTGGAGGCTGAAGATGTGGGCGTCTACTATTGCGTCCA |
| | | GGCAACCCAGTTCCCTCTGACATTTGGCGGGGGAACTA |
| | | AGGTGGAGATCAAGGGAGGAGGAGGATCTGGAGGAGG |
| | | AGGAAGTGGAGGAGGAGGATCCGAAGTGCAGCTGGTC |
| | | CAGTCTGGGGCCGAGGTGAAGAAACCTGGAGAAAGTCT |
| | | GAAGATCTCATGTAAAGGCTCCGGGTACTCTTTCACAA |
| | | GTTATTGGATTGGCTGGGTCCGACAGATGCCAGGAAAG |
| | | GGCCTGGAGTGGATGGGAATCATCTACCCCGGCGACAG |
| | | CGATACCCGGTATTCTCCTAGTTTTCAGGGCCAGGTGAC |
| | | AATCAGCGCAGACAAGTCCATTACCACAGCCTATCTGC |
| | | AGTGGTCAAGCCTGAAAGCCTCTGATACCGCTATGTAC |
| | | TATTGTGCCAGGCACCCTAGCTACGGGTCAGGAAGCCC |
| | | AAACTTTGACTATTGGGGCCAGGGGACACTGGTGACTG |
| | | TCTCCTCTACTACTACCCCAGCCCCACGTCCCCCCACGC |
| | | CAGCTCCAACGATAGCAAGTCAGCCCTTATCTCTTCGCC |
| | | CTGAGGCTTGCAGGCCCGCGGCGGGCGGCGCCGTTCAC |
| | | ACGCGAGGACTAGACTTCGCCTGCGACATCTACATCTG |
| | | GGCACCACTAGCCGGGACTTGCGGAGTGTTGTTGTTGA |
| | | GCTTGGTAATAACGCTCTACTGCAAACGCGGCAGCAGC |
| | | GAACTGCTGTATATTTTTAAACAGCCGTTTATGCGCCCG |
| | | GTGCAGACCACCAGCCAGCAGAACGGCTGCAGCTGCGA |
| | | ATTTCCGCAGCAGCAGCAGGGCGGCTGCGAACTGAGAG |
| | | TGAAGTTCTCTCGCTCCGCGGACGCACCCGCTTACCAGC |
| | | AGGGTCAGAACCAGCTATACAACGAGTTAAACCTGGGG |
| | | CGCCGGGAGGAGTACGACGTGTTAGACAAGCGTAGAG |
| | | GTAGGGACCCGGAGATGGGAGGCAAGCCTCGGAGAAA |
| | | GAACCCCCAGGAGGGCCTGTACAACGAACTCCAGAAGG |
| | | ACAAGATGGCTGAGGCGTACTCGGAGATTGGTATGAAG |
| | | GGCGAGAGACGTCGCGGAAAGGGACACGACGGCTTAT |
| | | ACCAGGGGCTTTCCACCGCGACCAAGGACACATACGAC |
| | | GCGCTGCACATGCAAGCCTTACCACCTCGATGA |
| 81 | huCD20 scFv | MALPVTALLLPLALLLHAARPDIVMTQTPLSSPVTLGQPAS |
| | CAR (CD8sp-VL- | ISCRSSQSLVYSDGNTYLSWLQQRPGQPPRLLIYKISNRFSG |
| | Linker-VH-CD8- | VPDRFSGSGAGTDFTLKISRVEAEDVGVYYCVQATQFPLT |
| | CD8-N1-CD3) | FGGGTKVEIKGGGGSGGGGSGGGGSEVQLVQSGAEVKKP |
| | | GESLKISCKGSGYSFTSYWIGWVRQMPGKGLEWMGIIYPG |
| | | DSDTRYSPSFQGQVTISADKSETTAYLQWSSLKASDTAMY |
| | | YCARHPSYGSGSPNFDYWGQGTLVTVSSTTTPAPRPPTPA |
| | | PTIASQPLSLRPEACRPAAGGAVHTRGLDFACDIYIWAPLA |
| | | GTCGVLLLSLVITLYCKHSRKKFVHLLKRPFIKTTGAAQM |
| | | EDASSCRCPQEEEGECDLRVKFSRSADAPAYQQGQNQLY |
| | | NELNLGRREEYDVLDKRRGRDPEMGGKPRRKNPQEGLYN |
| | | ELQKDKMAEAYSEIGMKGERRRGKGHDGLYQGLSTATK |
| | | DTYDALHMQALPPR |
| 82 | huCD20 scFv | ATGGCGCTCCCAGTGACAGCCTTACTTTTACCTCTGGCG |
| | CAR (CD8sp-VL- | TTATTATTGCACGCGGCTCGTCCTGACATTGTGATGACT |
| | Linker-VH-CD8- | CAGACACCACTGAGCTCCCCAGTGACTCTGGGACAGCC |
| | CD8-N1-CD3) | AGCCAGTATCTCATGCAGATCTAGTCAGTCACTGGTCTA |
| | | CAGCGACGGCAACACCTATCTGAGCTGGCTGCAGCAGC |
| | | GACCAGGACAGCCACCTAGACTGCTGATCTACAAGATT |
| | | TCCAATAGGTTCTCTGGAGTGCCCGACCGCTTTAGCGGA |
| | | TCCGGAGCTGGAACTGATTTCACCCTGAAAATCTCCCGC |
| | | GTGGAGGCTGAAGATGTGGGCGTCTACTATTGCGTCCA |
| | | GGCAACCCAGTTCCCTCTGACATTTGGCGGGGGAACTA |
| | | AGGTGGAGATCAAGGGAGGAGGAGGATCTGGAGGAGG |
| | | AGGAAGTGGAGGAGGAGGATCCGAAGTGCAGCTGGTC |
| | | CAGTCTGGGGCCGAGGTGAAGAAACCTGGAGAAAGTCT |
| | | GAAGATCTCATGTAAAGGCTCCGGGTACTCTTTCACAA |
| | | GTTATTGGATTGGCTGGGTCCGACAGATGCCAGGAAAG |
| | | GGCCTGGAGTGGATGGGAATCATCTACCCCGGCGACAG |
| | | CGATACCCGGTATTCTCCTAGTTTTCAGGGCCAGGTGAC |
| | | AATCAGCGCAGACAAGTCCATTACCACAGCCTATCTGC |
| | | AGTGGTCAAGCCTGAAAGCCTCTGATACCGCTATGTAC |
| | | TATTGTGCCAGGCACCCTAGCTACGGGTCAGGAAGCCC |
| | | AAACTTTGACTATTGGGGCCAGGGGACACTGGTGACTG |
| | | TCTCCTCTACTACTACCCCAGCCCCACGTCCCCCCACGC |
| | | CAGCTCCAACGATAGCAAGTCAGCCCTTATCTCTTCGCC |
| | | CTGAGGCTTGCAGGCCCGCGGCGGGCGGCGCCGTTCAC |
| | | ACGCGAGGACTAGACTTCGCCTGCGACATCTACATCTG |
| | | GGCACCACTAGCCGGGACTTGCGGAGTGTTGTTGTTGA |
| | | GCTTGGTAATAACGCTCTACTGCAAACATAGCCGCAAA |
| | | AAATTTGTGCATCTGCTGAAACGCCCGTTTATTAAAACC |
| | | ACCGGCGCGGCGCAGATGGAAGATGCGAGCAGCTGCCGC |
| | | TGCCCGCAGGAAGAAGAAGGCGAATGCGATCTGAGAGTG |
| | | AAGTTCTCTCGCTCCGCGGACGCACCCGCTTACCA |
| | | GCAGGGTCAGAACCAGCTATACAACGAGTTAAACCTGG |
| | | GGCGCCGGGAGGAGTACGACGTGTTAGACAAGCGTAG |
| | | AGGTAGGGACCCGGAGATGGGAGGCAAGCCTCGGAGA |
| | | AAGAACCCCCAGGAGGGCCTGTACAACGAACTCCAGAA |
| | | GGACAAGATGGCTGAGGCGTACTCGGAGATTGGTATGA |
| | | AGGGCGAGAGACGTCGCGGAAAGGGACACGACGGCTT |
| | | ATACCAGGGGCTTTCCACCGCGACCAAGGACACATACG |
| | | ACGCGCTGCACATGCAAGCCTTACCACCTCGATGA |

1. A polynucleotide encoding a chimeric antigen receptor, wherein said polynucleotide comprises a nucleic acid sequence set forth in any one of SEQ ID NO: 40, SEQ ID NO: 42, SEQ ID NO: 44, SEQ ID NO: 46, SEQ ID NO: 52, SEQ ID NO: 54, SEQ ID NO: 56, and SEQ ID NO: 58.

2. The polynucleotide of claim 1, wherein said chimeric antigen receptor further comprises a signal peptide.

3. The polynucleotide of claim 1 or claim 2, wherein said polynucleotide sequence is set forth in SEQ ID NO: 40.

4. The polynucleotide of claim 1 or claim 2, wherein said polynucleotide sequence is set forth in SEQ ID NO: 42.

5. The polynucleotide of claim 1 or claim 2, wherein said polynucleotide sequence is set forth in SEQ ID NO: 44.

6. The polynucleotide of claim 1 or claim 2, wherein said polynucleotide sequence is set forth in SEQ ID NO: 46.

7. The polynucleotide of claim 1 or claim 2, wherein said polynucleotide sequence is set forth in SEQ ID NO: 52.

8. The polynucleotide of claim 1 or claim 2, wherein said polynucleotide sequence is set forth in SEQ ID NO: 54.

9. The polynucleotide of claim 1 or claim 2, wherein said polynucleotide sequence is set forth in SEQ ID NO: 56.

10. The polynucleotide of claim 1 or claim 2, wherein said polynucleotide sequence is set forth in SEQ ID NO: 58.

11. The polynucleotide of any one of claims 1-10, wherein said polynucleotide is an mRNA.

12. A recombinant DNA construct comprising said polynucleotide of any one of claims 1-10.

13. The recombinant DNA construct of claim 12, wherein said recombinant DNA construct encodes a virus comprising said polynucleotide.

14. The recombinant DNA construct of claim 13, wherein said virus is an adenovirus, a lentivirus, a retrovirus, or an adeno-associated virus (AAV).

15. The recombinant DNA construct of claim 14, wherein said virus is a recombinant AAV.

16. A virus comprising said polynucleotide of any one of claims 1-10.

17. The virus of claim 16, wherein said virus is an adenovirus, a lentivirus, a retrovirus, or an adeno-associated virus (AAV).

18. The virus of claim 17, wherein said virus is a recombinant AAV.

19. A method of producing a genetically-modified T cell, said method comprising introducing into a T cell:
(a) a first nucleic acid comprising a polynucleotide encoding an engineered nuclease having specificity for a recognition sequence in the genome of said T cell, wherein said engineered nuclease is expressed in said T cell; and
(b) a template nucleic acid comprising said polynucleotide of any one of claims 1-10;
wherein said engineered nuclease generates a cleavage site at said recognition sequence,
and wherein said polynucleotide of any one of claims 1-10 is inserted into the genome at said cleavage site.

20. The method of claim 19, wherein said template nucleic acid is introduced into said T cell using a virus.

21. The method of claim 20, wherein said virus is a recombinant AAV vector.

22. The method of any one of claims 19-21, wherein said engineered nuclease is an engineered meganuclease, a zinc finger nuclease, a TALEN, a compact TALEN, a CRISPR system nuclease, or a megaTAL.

23. The method of any one of claims 19-22, wherein said engineered nuclease is an engineered meganuclease.

24. The method of any one of claims 19-23, wherein said T cell is a human T cell, or a cell derived therefrom.

25. A genetically-modified T cell prepared by the method of any one of claims 19-24.

26. A method of producing a genetically-modified T cell, said method comprising introducing into a T cell a nucleic acid comprising said polynucleotide of any one of claims 1-10, wherein said polynucleotide is introduced into said T cell by a lentivirus, and wherein said polynucleotide is randomly integrated into the genome of said T cell.

27. The method of claim 26, wherein said T cell has no detectable cell surface expression of an endogenous T cell receptor.

28. The method of claim 26 or 27, wherein said T cell is a human T cell, or a cell derived therefrom.

29. A genetically-modified T cell prepared by the method of any one of claims 26-28.

30. A genetically-modified T cell which expresses said chimeric antigen receptor encoded by the nucleic acid of any one of claims 1-10.

31. The genetically-modified T cell of claim 30, wherein said genetically-modified T cell is a genetically-modified human T cell, or a cell derived therefrom.

32. A genetically-modified T cell comprising in its genome said polynucleotide of any one of claims 1-10, wherein said polynucleotide expresses a chimeric antigen receptor and wherein said chimeric antigen receptor is expressed on the cell surface of said genetically-modified T cell.

33. The genetically-modified T cell of claim 32, wherein said polynucleotide is inserted into the genome of said genetically-modified T cell within a target gene, wherein expression of the polypeptide encoded by said target gene is disrupted.

34. The genetically-modified T cell of claim 33, wherein said target gene is a T cell receptor alpha constant region gene.

35. The genetically-modified T cell of claim 33 or 34, wherein said target gene is a T cell receptor alpha constant region gene, and wherein said genetically-modified cell has no detectable cell surface expression of an endogenous T cell receptor.

36. The genetically-modified T cell of any one of claims 32-35, wherein said genetically-modified T cell is a genetically-modified human T cell, or a cell derived therefrom.

37. A population of genetically-modified T cells comprising a plurality of said genetically-modified T cell of any one of claims 25 and 29-36.

38. The population of genetically-modified T cells of claim 37, wherein at least 30% of cells express said chimeric antigen receptor on their cell surface and have no detectable cell surface expression of an endogenous T cell receptor.

39. A pharmaceutical composition comprising a pharmaceutically-acceptable carrier and said population of cells of claim 37 or 38.

40. A pharmaceutical composition comprising a pharmaceutically-acceptable carrier and said genetically-modified T cell of any one of claims 25 and 29-36.

41. A pharmaceutical composition comprising a pharmaceutically-acceptable carrier and a genetically-modified T cell, wherein the genetically-modified T cell comprises the virus of any one of claims 16-18.

42. A pharmaceutical composition comprising a pharmaceutically-acceptable carrier and a genetically-modified T cell, wherein the genetically-modified T cell comprises the recombinant DNA construct of any one of claims 12-15.

43. A pharmaceutical composition comprising a pharmaceutically-acceptable carrier and a genetically-modified T cell, wherein the genetically-modified T cell comprises a polynucleotide according to any one of claims 1-10 and is capable of expressing said chimeric antigen receptor.

44. A pharmaceutical composition comprising a pharmaceutically-acceptable carrier and a genetically-modified T cell, wherein the genetically-modified T cell comprises said polynucleotide of any one of claims 1-10.

45. A method of immunotherapy for treating cancer in subject in need thereof, said method comprising administering to said subject an effective amount of said pharmaceutical composition of any one of claims 39-44.

46. The method of claim 45, wherein the subject is suffering from a cancer of B-cell origin.

47. The method of claim 45, wherein said cancer is selected from the group consisting of B-lineage acute lymphoblastic leukemia, B-cell chronic lymphocytic leukemia and B-cell non-Hodgkin lymphoma.

48. The method of claim 45, wherein said cancer is selected from the group consisting of lung cancer, melanoma, breast cancer, prostate cancer, colon cancer, renal cell carcinoma, ovarian cancer, neuroblastoma, rhabdomyosarcoma, leukemia and lymphoma, acute lymphoblastic leukemia, small cell lung cancer, Hodgkin's lymphoma, and childhood acute lymphoblastic leukemia.

49. The method of claim 45, wherein said pharmaceutical composition is administered in combination with a cancer therapy selected from the group consisting of chemotherapy, surgery, radiation, and gene therapy.

50. A method of treating cancer in subject in need thereof comprising administering to the individual a composition comprising a population of genetically-modified cells, wherein said cells express at least one polynucleotide encoding at least one chimeric antigen receptor according to any one of claims 1-10.

51. The method of claim 50, wherein said cells express polynucleotides encoding at least two chimeric antigen receptors according to any one of claims 1-10.

52. The method of claim 50, wherein the subject is suffering from a cancer of B-cell origin.

53. The method of claim 50, wherein said cancer is selected from the group consisting of B-lineage acute lymphoblastic leukemia, B-cell chronic lymphocytic leukemia and B-cell non-Hodgkin lymphoma.

54. The method of claim 50, wherein said cancer is selected from the group consisting of lung cancer, melanoma, breast cancer, prostate cancer, colon cancer, renal cell carcinoma, ovarian cancer, neuroblastoma, rhabdomyosarcoma, leukemia and lymphoma, acute lymphoblastic leukemia, small cell lung cancer, Hodgkin lymphoma, and childhood acute lymphoblastic leukemia.

55. The method of claim 50, wherein said pharmaceutical composition is administered in combination with a cancer therapy selected from the group consisting of chemotherapy, surgery, radiation, and gene therapy.

56. A method for treating cancer in a subject in need thereof, said method comprising administering to the subject genetically-modified human T cells expressing a chimeric antigen receptor (CAR) that is encoded by a polynucleotide according to any one of claims 1-10.

57. The method of claim 56, wherein the subject is suffering from a cancer of B-cell origin.

58. The method of claim 56, wherein said cancer is selected from the group consisting of B-lineage acute lymphoblastic leukemia, B-cell chronic lymphocytic leukemia and B-cell non-Hodgkin lymphoma.

59. The method of claim 56, wherein said cancer is selected from the group consisting of lung cancer, melanoma, breast cancer, prostate cancer, colon cancer, renal cell carcinoma, ovarian cancer, neuroblastoma, rhabdomyosarcoma, leukemia and lymphoma, acute lymphoblastic leukemia, small cell lung cancer, Hodgkin lymphoma, and childhood acute lymphoblastic leukemia.

60. The method of claim 56, wherein said pharmaceutical composition is administered in combination with a cancer therapy selected from the group consisting of chemotherapy, surgery, radiation, and gene therapy.

61. A kit comprising a container comprising the polynucleotide of any one of claims 1-10, with reagents and/or instructions for use.


SEQUENCE LISTING (82 sequences)

SEQ ID NO: 1 - protein, 122 aa, Artificial Sequence
  Synthesized
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 2 - DNA, 366 bp, Artificial Sequence
  Synthesized
gaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctca

SEQ ID NO: 3 - protein, 106 aa, Artificial Sequence
  Synthesized
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 4 - DNA, 318 bp, Artificial Sequence
  Synthesized
gacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaa

SEQ ID NO: 5 - protein, 122 aa, Artificial Sequence
  Synthesized
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 6 - DNA, 366 bp, Artificial Sequence
  Synthesized
gaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctct

SEQ ID NO: 7 - protein, 112 aa, Artificial Sequence
  Synthesized
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 8 - DNA, 336 bp, Artificial Sequence
  Synthesized
gacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaag

SEQ ID NO: 9 - protein, 5 aa, Artificial Sequence
  Synthesized
SYNMH

SEQ ID NO: 10 - protein, 17 aa, Artificial Sequence
  Synthesized
AIYPGNGDTSYNQKFKG

SEQ ID NO: 11 - protein, 13 aa, Artificial Sequence
  Synthesized
SNYYGSSYWFFDV

SEQ ID NO: 12 - protein, 10 aa, Artificial Sequence
  Synthesized
RASSSVNYMD

SEQ ID NO: 13 - protein, 7 aa, Artificial Sequence
  Synthesized
ATSNLAS

SEQ ID NO: 14 - protein, 9 aa, Artificial Sequence
  Synthesized
QQWSFNPPT

SEQ ID NO: 15 - protein, 5 aa, Artificial Sequence
  Synthesized
SYWIG

SEQ ID NO: 16 - protein, 17 aa, Artificial Sequence
  Synthesized
IIYPGDSDTRYSPSFQG

SEQ ID NO: 17 - protein, 13 aa, Artificial Sequence
  Synthesized
HPSYGSGSPNFDY

SEQ ID NO: 18 - protein, 16 aa, Artificial Sequence
  Synthesized
RSSQSLVYSDGNTYLS

SEQ ID NO: 19 - protein, 7 aa, Artificial Sequence
  Synthesized
KISNRFS

SEQ ID NO: 20 - protein, 9 aa, Artificial Sequence
  Synthesized
VQATQFPLT

SEQ ID NO: 21 - protein, 42 aa, Artificial Sequence
  Synthesized
KHSRKKFVHLLKRPFIKTTGAAQMEDASSCRCPQEEEGECDL

SEQ ID NO: 22 - DNA, 126 bp, Artificial Sequence
  Synthesized
aaacatagccgcaaaaaatttgtgcatctgctgaaacgcccgtttattaaaaccaccggcgcggcgcagatggaagatgcgagcagctgccgctgcccgcaggaagaagaaggcgaatgcgatctg

SEQ ID NO: 23 - protein, 42 aa, Artificial Sequence
  Synthesized
KASRKKAAAAAKSPFASPASSAQEEDASSCRAPSEEEGSCEL

SEQ ID NO: 24 - DNA, 126 bp, Artificial Sequence
  Synthesized
aaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactg

SEQ ID NO: 25 - protein, 15 aa, Artificial Sequence
  Synthesized
GGGGSGGGGSGGGGS

SEQ ID NO: 26 - DNA, 45 bp, Artificial Sequence
  Synthesized
ggaggaggaggatctggaggaggaggaagtggaggaggaggatcc

SEQ ID NO: 27 - protein, 24 aa, Artificial Sequence
  Synthesized
TTTPAPRPPTPAPTIASQPLSLRP

SEQ ID NO: 28 - DNA, 72 bp, Artificial Sequence
  Synthesized
actactaccccagccccacgtccccccacgccagctccaacgatagcaagtcagcccttatctcttcgccct

SEQ ID NO: 29 - protein, 45 aa, Artificial Sequence
  Synthesized
EACRPAAGGAVHTRGLDFACDIYIWAPLAGTCGVLLLSLVITLYC

SEQ ID NO: 30 - DNA, 135 bp, Artificial Sequence
  Synthesized
gaggcttgcaggcccgcggcgggcggcgccgttcacacgcgaggactagacttcgcctgcgacatctacatctgggcaccactagccgggacttgcggagtgttgttgttgagcttggtaataacgctctactgc

SEQ ID NO: 31 - protein, 112 aa, Artificial Sequence
  Synthesized
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 336 bp, Artificial Sequence
  Synthesized
agagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 33 - protein, 21 aa, Artificial Sequence
  Synthesized
MALPVTALLLPLALLLHAARP

SEQ ID NO: 34 - DNA, 63 bp, Artificial Sequence
  Synthesized
atggcgctcccagtgacagccttacttttacctctggcgttattattgcacgcggctcgtcct

SEQ ID NO: 35 - protein, 246 aa, Artificial Sequence
  Synthesized
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 36 - DNA, 738 bp, Artificial Sequence
  Synthesized
gacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctca

SEQ ID NO: 37 - protein, 246 aa, Artificial Sequence
  Synthesized
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 38 - DNA, 738 bp, Artificial Sequence
  Synthesized
gaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaa

SEQ ID NO: 39 - protein, 469 aa, Artificial Sequence
  Synthesized
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSTTTPAPRPPTPAPTIASQPLSLRPEACRPAAGGAVHTRGLDFACDIYIWAPLAGTCGVLLLSLVITLYCKHSRKKFVHLLKRPFIKTTGAAQMEDASSCRCPQEEEGECDLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1407 bp, Artificial Sequence
  Synthesized
gacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaacatagccgcaaaaaatttgtgcatctgctgaaacgcccgtttattaaaaccaccggcgcggcgcagatggaagatgcgagcagctgccgctgcccgcaggaagaagaaggcgaatgcgatctgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 41 - protein, 469 aa, Artificial Sequence
  Synthesized
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCKHSRKKFVHLLKRPFIKTTGAAQMEDASSCRCPQEEEGECDLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1407 bp, Artificial Sequence
  Synthesized
gaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaacatagccgcaaaaaatttgtgcatctgctgaaacgcccgtttattaaaaccaccggcgcggcgcagatggaagatgcgagcagctgccgctgcccgcaggaagaagaaggcgaatgcgatctgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 43 - protein, 469 aa, Artificial Sequence
  Synthesized
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 1407 bp, Artificial Sequence
  Synthesized
gacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 45 - protein, 469 aa, Artificial Sequence
  Synthesized
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGSTSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 1407 bp, Artificial Sequence
  Synthesized
gaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaggcagtactagcggtggtggctccgggggcggttccggtgggggcggcagcagcgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 47 - protein, 249 aa, Artificial Sequence
  Synthesized
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 48 - DNA, 747 bp, Artificial Sequence
  Synthesized
gacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagggaggaggaggatctggaggaggaggaagtggaggaggaggatccgaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctct

SEQ ID NO: 49 - protein, 249 aa, Artificial Sequence
  Synthesized
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSGGGGSGGGGSGGGGSDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 50 - DNA, 747 bp, Artificial Sequence
  Synthesized
gaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctggaggaggaggatctggaggaggaggaagtggaggaggaggatccgacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaag

SEQ ID NO: 51 - protein, 472 aa, Artificial Sequence
  Synthesized
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKHSRKKFVHLLKRPFIKTTGAAQMEDASSCRCPQEEEGECDLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 1416 bp, Artificial Sequence
  Synthesized
gacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagggaggaggaggatctggaggaggaggaagtggaggaggaggatccgaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaacatagccgcaaaaaatttgtgcatctgctgaaacgcccgtttattaaaaccaccggcgcggcgcagatggaagatgcgagcagctgccgctgcccgcaggaagaagaaggcgaatgcgatctgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 53 - protein, 472 aa, Artificial Sequence
  Synthesized
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSGGGGSGGGGSGGGGSDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKTTTPAPRPPTPAPTIASQPLSLRPEACRPAAGGAVHTRGLDFACDIYIWAPLAGTCGVLLLSLVITLYCKHSRKKFVHLLKRPFIKTTGAAQMEDASSCRCPQEEEGECDLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 1416 bp, Artificial Sequence
  Synthesized
gaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctggaggaggaggatctggaggaggaggaagtggaggaggaggatccgacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaacatagccgcaaaaaatttgtgcatctgctgaaacgcccgtttattaaaaccaccggcgcggcgcagatggaagatgcgagcagctgccgctgcccgcaggaagaagaaggcgaatgcgatctgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 55 - protein, 472 aa, Artificial Sequence
  Synthesized
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 1416 bp, Artificial Sequence
  Synthesized
gacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagggaggaggaggatctggaggaggaggaagtggaggaggaggatccgaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 57 - protein, 472 aa, Artificial Sequence
  Synthesized
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSGGGGSGGGGSGGGGSDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1416 bp, Artificial Sequence
  Synthesized
gaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctggaggaggaggatctggaggaggaggaagtggaggaggaggatccgacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcga

SEQ ID NO: 59 - protein, 46 aa, Artificial Sequence
  Synthesized
AKPTTTPAPRPPTPAPTIASQPLSLRPEACRPAAGGAVHTRGLDFA

SEQ ID NO: 60 - protein, 40 aa, Artificial Sequence
  Synthesized
KIEVMYPPPYLDNEKSNGTIIHVKGKHLCPSPLFPGPSKP

SEQ ID NO: 61 - protein, 88 aa, Artificial Sequence
  Synthesized
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 62 - protein, 28 aa, Artificial Sequence
  Synthesized
LDPKLCYLLDGILFIYGVILTALFLRVK

SEQ ID NO: 63 - protein, 21 aa, Artificial Sequence
  Synthesized
LCYLLDGILFIYGVILTALFL

SEQ ID NO: 64 - protein, 27 aa, Artificial Sequence
  Synthesized
FWVLVVVGGVLACYSLLVTVAFIIFWV

SEQ ID NO: 65 - protein, 297 aa, Homo Sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 66 - DNA, 22 bp, Homo Sapiens
tggcctggagcaacaaatctga

SEQ ID NO: 67 - DNA, 22 bp, Homo Sapiens
accggacctcgttgtttagact

SEQ ID NO: 68 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 69 - protein, 354 aa, Artificial Sequence
  Synthesized
MNTKYNKEFLLYLAGFVDGDGSIYACIYPHQRAKFKHLLKLVFAVHQRTTRRWFLDKLVDEIGVGYVYDIGSVSEYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIAPRQGSKFKHRLKLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 70 - protein, 354 aa, Artificial Sequence
  Synthesized
MNTKYNKEFLLYLAGFVDGDGSIFASIYPHQRAKFKHFLKLTFAVYQKTQRRWFLDKLVDEIGVGYVYDSGSVSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIAPRQGSKFKHRLKLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 71 - protein, 18 aa, Artificial Sequence
  Synthesized
GSTSGGGSGGGSGGGGSS

SEQ ID NO: 72 - DNA, 54 bp, Artificial Sequence
  Synthesized
ggcagtactagcggtggtggctccgggggcggttccggtgggggcggcagcagc

SEQ ID NO: 73 - protein, 490 aa, Artificial Sequence
  Synthesized
MALPVTALLLPLALLLHAARPDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1473 bp, Artificial Sequence
  Synthesized
atggcgctcccagtgacagccttacttttacctctggcgttattattgcacgcggctcgtcctgacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcaccgtctcctcaactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatga

SEQ ID NO: 75 - protein, 493 aa, Artificial Sequence
  Synthesized
MALPVTALLLPLALLLHAARPDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKASRKKAAAAAKSPFASPASSAQEEDASSCRAPSEEEGSCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - DNA, 1482 bp, Artificial Sequence
  Synthesized
atggcgctcccagtgacagccttacttttacctctggcgttattattgcacgcggctcgtcctgacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagggaggaggaggatctggaggaggaggaagtggaggaggaggatccgaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaagcgagccgcaaaaaagcggcggcggcggcgaaaagcccgtttgcgagcccggcgagcagcgcgcaggaagaagatgcgagcagctgccgcgcgccgagcgaagaagaaggcagctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatga

SEQ ID NO: 77 - protein, 493 aa, Artificial Sequence
  Synthesized
MALPVTALLLPLALLLHAARPDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - DNA, 1482 bp, Artificial Sequence
  Synthesized
atggcgctcccagtgacagccttacttttacctctggcgttattattgcacgcggctcgtcctgacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagggaggaggaggatctggaggaggaggaagtggaggaggaggatccgaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaagcgtgggagaaagaagctcttgtacattttcaagcagccattcatgcgtcccgttcagacgactcaggaggaggacggctgctcgtgccgattcccggaggaggaggagggcggttgcgaactcagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatga

SEQ ID NO: 79 - protein, 493 aa, Artificial Sequence
  Synthesized
MALPVTALLLPLALLLHAARPDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKRGSSELLYIFKQPFMRPVQTTSQQNGCSCEFPQQQQGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 1482 bp, Artificial Sequence
  Synthesized
atggcgctcccagtgacagccttacttttacctctggcgttattattgcacgcggctcgtcctgacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagggaggaggaggatctggaggaggaggaagtggaggaggaggatccgaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaacgcggcagcagcgaactgctgtatatttttaaacagccgtttatgcgcccggtgcagaccaccagccagcagaacggctgcagctgcgaatttccgcagcagcagcagggcggctgcgaactgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatga

SEQ ID NO: 81 - protein, 493 aa, Artificial Sequence
  Synthesized
MALPVTALLLPLALLLHAARPDIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIKGGGGSGGGGSGGGGSEVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSSTTTPAPRPPTPAPTIASQPLSLRPEACRPAAGGAVHTRGLDFACDIYIWAPLAGTCGVLLLSLVITLYCKHSRKKFVHLLKRPFIKTTGAAQMEDASSCRCPQEEEGECDLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 1482 bp, Artificial Sequence
  Synthesized
atggcgctcccagtgacagccttacttttacctctggcgttattattgcacgcggctcgtcctgacattgtgatgactcagacaccactgagctccccagtgactctgggacagccagccagtatctcatgcagatctagtcagtcactggtctacagcgacggcaacacctatctgagctggctgcagcagcgaccaggacagccacctagactgctgatctacaagatttccaataggttctctggagtgcccgaccgctttagcggatccggagctggaactgatttcaccctgaaaatctcccgcgtggaggctgaagatgtgggcgtctactattgcgtccaggcaacccagttccctctgacatttggcgggggaactaaggtggagatcaagggaggaggaggatctggaggaggaggaagtggaggaggaggatccgaagtgcagctggtccagtctggggccgaggtgaagaaacctggagaaagtctgaagatctcatgtaaaggctccgggtactctttcacaagttattggattggctgggtccgacagatgccaggaaagggcctggagtggatgggaatcatctaccccggcgacagcgatacccggtattctcctagttttcagggccaggtgacaatcagcgcagacaagtccattaccacagcctatctgcagtggtcaagcctgaaagcctctgataccgctatgtactattgtgccaggcaccctagctacgggtcaggaagcccaaactttgactattggggccaggggacactggtgactgtctcctctactactaccccagccccacgtccccccacgccagctccaacgatagcaagtcagcccttatctcttcgccctgaggcttgcaggcccgcggcgggcggcgccgttcacacgcgaggactagacttcgcctgcgacatctacatctgggcaccactagccgggacttgcggagtgttgttgttgagcttggtaataacgctctactgcaaacatagccgcaaaaaatttgtgcatctgctgaaacgcccgtttattaaaaccaccggcgcggcgcagatggaagatgcgagcagctgccgctgcccgcaggaagaagaaggcgaatgcgatctgagagtgaagttctctcgctccgcggacgcacccgcttaccagcagggtcagaaccagctatacaacgagttaaacctggggcgccgggaggagtacgacgtgttagacaagcgtagaggtagggacccggagatgggaggcaagcctcggagaaagaacccccaggagggcctgtacaacgaactccagaaggacaagatggctgaggcgtactcggagattggtatgaagggcgagagacgtcgcggaaagggacacgacggcttataccaggggctttccaccgcgaccaaggacacatacgacgcgctgcacatgcaagccttaccacctcgatga